(19) United States
(12) Patent Application Publication
(10) Pub. No.: US 2021/0137903 A1
(43) Pub. Date: May 13, 2021

(54) ANTI-ANDROGENS FOR THE TREATMENT OF PROSTRATE CANCER

(71) Applicant: Aragon Pharmaceuticals, Inc., Los Angeles, CA (US)

(72) Inventors: Caly Chien, Spring House, PA (US); Maura Erba, Latina (IT); Meike Van Uffel, Beerse (BE); Alex Yu, Ambler, PA (US); Anasuya Hazra, Furlong, PA (US)

(21) Appl. No.: 16/930,487

(22) Filed: Jul. 16, 2020

Related U.S. Application Data
(63) Continuation-in-part of application No. 16/906,327, filed on Jun. 19, 2020, now abandoned.
(60) Provisional application No. 62/790,561, filed on Jan. 10, 2019.

(30) Foreign Application Priority Data
Jan. 10, 2020 (IB) PCT/IB2020/050194

(51) Int. Cl. A61K 31/4439; A23L 33/10; A23L 29/00; A61K 9/00; A61P 35/04

(52) U.S. Cl. CPC A61K 31/4439; A23L 33/10; A61P 35/04; A61K 9/0095; A23L 29/045

(57) ABSTRACT

Disclosed herein are pharmaceutical compositions and methods of treating prostate cancer including castrate resistant prostate cancer, metastatic castration resistant prostate cancer and non-metastatic castration resistant prostate cancer with an approved drug product containing an anti-androgen selected from the group consisting of enzalutamide, apalutamide and darolutamide.

### CROSS REFERENCE TO RELATED APPLICATIONS

This application is a continuation-in-part of U.S. patent application Ser. No. 16/906,327, filed on Jun. 19, 2020; which is the national stage application, filed under 35 U.S.C. § 371, of International Patent Application No. PCT/IB2020/050194, filed on Jan. 10, 2020; which application claims the benefit of U.S. Provisional Application 62/790,561, filed on Jan. 10, 2019, U.S. Provisional Application 62/790,566, filed on Jan. 10, 2019, U.S. Provisional Application 62/790,567, filed on Jan. 10, 2019, and U.S. Provisional Application 62/790,574, filed on Jan. 10, 2019, which are incorporated by reference herein in their entirety.

### FIELD OF THE INVENTION

Disclosed herein are pharmaceutical compositions and methods of treating prostate cancer, including castrate resistant prostate cancer, metastatic castration resistant prostate cancer, metastatic castration sensitive prostate cancer and non-metastatic castration resistant prostate cancer with an approved drug product containing an anti-androgen selected from the group consisting of enzalutamide, apalutamide and darolutamide.

### BACKGROUND OF THE INVENTION

Prostate cancer is the second most frequently diagnosed cancer and the sixth leading cause of cancer death in males, accounting for 14% (903,500) of the total new cancer cases and 6% (258,400) of the total cancer deaths in males worldwide. The course of prostate cancer from diagnosis to death is best categorized as a series of clinical stages based on the extent of disease, hormonal status, and absence or presence of detectable metastases: localized disease, rising levels of prostate-specific antigen (PSA) after radiation therapy or surgery with no detectable metastases, and clinical metastases in the non-castrate or castrate stage. Although surgery, radiation, or a combination of both can be curative for patients with localized disease, a significant proportion of these patients have recurrent disease as evidenced by a rising level of PSA, which can lead to the development of metastases, especially in the high-risk group—a transition to the lethal stage of the disease.

Androgen depletion is the standard treatment with a generally predictable outcome: decline in PSA, a period of stability in which the tumor does not proliferate, followed by rising PSA and regrowth as castration-resistant disease. Historically, ADT has been the standard of care for patients with metastatic prostate cancer.

Molecular profiling studies of castration-resistance prostate cancers commonly show increased androgen receptor (AR) expression, which can occur through AR gene amplification or other mechanisms.

There is a need for a next-generation AR antagonist that overcome the potential therapeutic deficiencies of existing therapies. The disclosed methods are directed to these and other important needs.

### SUMMARY OF THE INVENTION

The present invention is directed to methods of treating prostate cancer including castrate resistant prostate cancer, metastatic castration resistant prostate cancer, metastatic castration sensitive prostate cancer and non-metastatic castration resistant prostate cancer comprising, consisting or consisting essentially of administer a safe and effective amount of anti-androgen (including but not limited to enzalutamide, apalutamide and darolutamide, preferably apalutamide), to a male human with a non-metastatic castration-resistant prostate cancer.

In an embodiment, the present invention is directed to a method of prostate cancer including castrate resistant prostate cancer, metastatic castration resistant prostate cancer, metastatic castration sensitive prostate cancer, and non-metastatic castration resistant prostate cancer comprising administering apalutamide to a male human with a non-metastatic castration-resistant prostate cancer; wherein the apalutamide is dispersed in a suitable vehicle.

In an embodiment, the present invention is directed to a method of prostate cancer including castrate resistant prostate cancer, metastatic castration resistant prostate cancer, metastatic castration sensitive prostate cancer, and non-metastatic castration resistant prostate cancer comprising administering apalutamide to a male human with a non-metastatic castration-resistant prostate cancer; wherein the apalutamide is dispersed in fluid or semi-solid, preferably a liquid or semi-solid.

In an embodiment, the present invention is directed to a method of prostate cancer including castrate resistant prostate cancer, metastatic castration resistant prostate cancer, metastatic castration sensitive prostate cancer, and non-metastatic castration resistant prostate cancer comprising administering apalutamide to a male human with a non-metastatic castration-resistant prostate cancer; wherein the apalutamide is dispersed in a semisolid selected from the group consisting of applesauce, fruit puree, yogurt and jam; preferably applesauce or yogurt.

In an embodiment, the present invention is directed to a method of prostate cancer including castrate resistant prostate cancer, metastatic castration resistant prostate cancer, metastatic castration sensitive prostate cancer, and non-metastatic castration resistant prostate cancer comprising administering apalutamide to a male human with a non-metastatic castration-resistant prostate cancer; wherein the apalutamide is dispersed in applesauce.

In an embodiment, the present invention is directed to a method of prostate cancer including castrate resistant prostate cancer, metastatic castration resistant prostate cancer, metastatic castration sensitive prostate cancer, and non-metastatic castration resistant prostate cancer comprising administering apalutamide to a male human with a non-metastatic castration-resistant prostate cancer; wherein the apalutamide is dispersed in a liquid selected from the group consisting of water, tea, coffee, milk, coconut milk, fruit juice, lemonade, plant based milk, sports drink and electrolyte drink; preferably, the liquid is water, tea or fruit juice.

In another embodiment, the present invention is directed to a method of prostate cancer including castrate resistant prostate cancer, metastatic castration resistant prostate cancer and non-metastatic castration resistant prostate cancer, wherein the non-metastatic castration-resistant prostate cancer is a high risk non-metastatic castration-resistant prostate cancer.

In another embodiment, the present invention is directed to a method of treating prostate cancer including castrate resistant prostate cancer, metastatic castration resistant prostate cancer, metastatic castration sensitive prostate cancer and non-metastatic castration resistant prostate cancer, wherein the apalutamide is administered in an amount between about 60 mg and about 240 mg. In another embodiment, the present invention is directed to a method of treating non-metastatic castration-resistant prostate cancer, wherein the apalutamide is administered in an amount of about 240 mg.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein the apalutamide is administered to a fasting male human.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein the apalutamide is dispersed by admixing one to four (preferably four) 60 mg solid tablets of apalutamide in a suitable vehicle.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein the apalutamide is dispersed by admixing one to four (preferably four) 60 mg solid tablets of apalutamide in between about 50 mL and about 200 mL or between about 50 g and about 200 g of a suitable vehicle.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein the apalutamide is dispersed by admixing four 60 mg solid tablets of apalutamide in about 120 mL of applesauce.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein the apalutamide is dispersed by admixing one to four (preferably four) 60 mg solid tablets of apalutamide in between about 50 g and about 200 g yogurt.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein the apalutamide is dispersed by admixing one to four (preferably four) 60 mg solid tablets of apalutamide in between about 50 mL and about 200 mL of tea, preferably green tea.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein the apalutamide is dispersed by admixing one to four (preferably four) 60 mg solid tablets of apalutamide in between about 50 mL and about 200 mL fruit juice, preferably orange juice.

In another embodiment, the present invention is directed to a method of treating prostate cancer including castrate resistant prostate cancer, metastatic castration resistant prostate cancer, metastatic castration sensitive prostate cancer and non-metastatic castration resistant prostate cancer, wherein the apalutamide dispersed in a suitable vehicle (preferably a liquid or semisolid such as applesauce, yogurt, tea, fruit juice, and the like) is administered over a period of about 5 minutes.

In another embodiment, the present invention is directed to a method of treating prostate cancer including castrate resistant prostate cancer, metastatic castration resistant prostate cancer, metastatic castration sensitive prostate cancer and non-metastatic castration resistant prostate cancer, wherein the apalutamide dispersed in applesauce is administered over a period of about 5 minutes.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein apalutamide dispersed in a suitable vehicle (preferably a liquid or semisolid such as applesauce, yogurt, tea, fruit juice, and the like) is administered daily.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein apalutamide dispersed in applesauce is administered daily.

In another embodiment, the present invention is directed to a method of treating prostate cancer including castrate resistant prostate cancer, metastatic castration resistant prostate cancer and non-metastatic castration resistant prostate cancer, wherein apalutamide dispersed in a suitable vehicle (preferably a liquid or semisolid such as applesauce, yogurt, fruit juice, tea, and the like) is administered orally on a continuous daily dosage schedule.

In another embodiment, the present invention is directed to a method of treating prostate cancer including castrate resistant prostate cancer, metastatic castration resistant prostate cancer and non-metastatic castration resistant prostate cancer, wherein apalutamide dispersed in applesauce is administered orally on a continuous daily dosage schedule.

In another embodiment, the present invention is directed to a method of treating prostate cancer including castrate resistant prostate cancer, metastatic castration resistant prostate cancer, metastatic castration sensitive prostate cancer and non-metastatic castration resistant prostate cancer, wherein the apalutamide dispersed in applesauce is administered in an amount and over a time period such that measured Cmax at median Tmax is achieved at between about 2 and about 3 hours. In another embodiment, the present invention is directed to a method of treating non-metastatic castration-resistant prostate cancer, wherein the apalutamide dispersed in applesauce is administered in an amount and over a time period such that measured Cmax is about 2.4 μg/mL. In another embodiment, the present invention is directed to a method of treating non-metastatic castration-resistant prostate cancer, wherein the apalutamide dispersed in applesauce is administered in an amount and over a time period such that measured AUC is about 225 μg*h/mL.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein a male human has said non-metastatic castration-resistant prostate cancer and has a prostate-specific antigen doubling time (PSADT) that is less than or equal to 10 months.

In another embodiment, the present invention is directed to a method of treating prostate cancer including castrate resistant prostate cancer, metastatic castration resistant prostate cancer, metastatic castration sensitive prostate cancer and non-metastatic castration resistant prostate cancer, wherein administration of the apalutamide provides an increase in the metastasis-free survival of a male human. In another embodiment, the present invention is directed to a method of treating non-metastatic castration-resistant prostate cancer, wherein administration of the apalutamide provides improved anti-tumor activity as measured by time to metastasis (TTM), progression-free survival (PFS) rate, time to symptomatic progression, overall survival (OS) rate, or time to initiation of cytotoxic chemotherapy. In another embodiment, the present invention is directed to a method of treating non-metastatic castration-resistant prostate cancer, wherein administration of the apalutamide results in no more than a grade 3 adverse event.

In another embodiment, the present invention is directed to a method of treating prostate cancer including castrate resistant prostate cancer, metastatic castration resistant prostate cancer, metastatic castration sensitive prostate cancer and non-metastatic castration resistant prostate cancer, wherein a male human having said non-metastatic castration-resistant prostate cancer is treatment naïve.

In another embodiment, the present invention is directed to a method of treating prostate cancer including castrate resistant prostate cancer, metastatic castration resistant prostate cancer, metastatic castration sensitive prostate cancer and non-metastatic castration resistant prostate cancer, wherein a male human having said nonmetastatic castration-resistant prostate cancer has received at least one prior therapy for the treatment of cancer. In another embodiment, the present invention is directed to a method of treating non-metastatic castration-resistant prostate cancer, wherein the prior therapy for the treatment of cancer is bicalutamide or flutamide.

In another embodiment, the present invention is directed to a method of treating prostate cancer including castrate resistant prostate cancer, metastatic castration resistant prostate cancer, metastatic castration sensitive prostate cancer, and non-metastatic castration resistant prostate cancer, wherein apalutamide is co-administered with one or more additional cancer treatments.

In another embodiment, the present invention is directed to a method of treating prostate cancer including castrate resistant prostate cancer, metastatic castration resistant prostate cancer, metastatic castration sensitive prostate cancer, metastatic castration sensitive prostate cancer, and non-metastatic castration resistant prostate cancer, wherein the amount of apalutamide administered is reduced when co-administered with one or more of:

(a) a CYP2C8 inhibitor, preferably gemfibrozil or clopidogrel; or

(b) a CYP3A4 inhibitor, preferably ketoconazole or ritonavir.

In another embodiment, the present invention is directed to a method of treating prostate cancer including castrate resistant prostate cancer, metastatic castration resistant prostate cancer, metastatic castration sensitive prostate cancer and non-metastatic castration resistant prostate cancer, wherein apalutamide is not co-administered with:

(a) medications that are primarily metabolized by CYP3A4, preferably darunavir, felodipine, midazolam or simvastatin;

(b) medications that are primarily metabolized by CYP2C19, preferably diazepam or omeprazole;

(c) medications that are primarily metabolized by CYP2C9, preferably warfarin or phenytoin; or

(d) medications that are substrates of UGT, preferably levothyroxine or valproic acid. In another embodiment, the present invention is directed to a method of treating non-metastatic castration-resistant prostate cancer, wherein apalutamide is not co-administered with:

(a) medications that are P-gp substrates, preferably fexofenadine, colchicine, dabigatran etexilate or digoxin; or

(b) BCRP/OATP1B1 substrates, preferably lapatinib, methotrexate, rosuvastatin, or repaglinide.

In an embodiment, the present invention is directed to a pharmaceutical composition comprising apalutamide dispersed in a suitable vehicle (preferably a liquid or semisolid such as applesauce, yogurt, fruit juice, tea, and the like). In another embodiment, the present invention is directed to a pharmaceutical composition comprising apalutamide dispersed in a suitable vehicle (preferably a liquid or semisolid such as applesauce, yogurt, fruit juice, tea, and the like), wherein the apalutamide is present in amount between about 60 mg an about 240 mg. In another embodiment, the present invention is directed to a pharmaceutical composition comprising apalutamide dispersed in a suitable vehicle (preferably a liquid or semisolid such as applesauce, yogurt, fruit juice, tea, and the like), wherein the apalutamide is present in amount of between about 60 g and about 240 mg, preferably about 240 mg.

In an embodiment, the present invention is directed to a pharmaceutical composition comprising apalutamide dispersed in applesauce. In another embodiment, the present invention is directed to a pharmaceutical composition comprising apalutamide dispersed in applesauce, wherein the apalutamide is present in amount between about 60 mg an about 240 mg. In another embodiment, the present invention is directed to a pharmaceutical composition comprising apalutamide dispersed in applesauce, wherein the apalutamide is present in amount of about 240 mg.

In another embodiment, the present invention is directed to a method for the preparation of a pharmaceutical composition comprising apalutamide dispersed in a suitable vehicle (preferably a liquid or semisolid such as applesauce, yogurt, fruit juice, tea, and the like), wherein the apalutamide is dispersed in the suitable vehicle by admixing one to four (preferably four) 60 mg solid tablets of apalutamide in between about 50 mL and about 200 mL or between about 50 g and about 200 g of the suitable vehicle.

In another embodiment, the present invention is directed to a method for the preparation of a pharmaceutical composition comprising apalutamide dispersed in applesauce, wherein the apalutamide is dispersed in the applesauce by admixing four 60 mg solid tablets of apalutamide in about 120 mL of applesauce.

In another embodiment, the present invention is directed to a method for the preparation of a pharmaceutical composition comprising apalutamide dispersed in applesauce, wherein the apalutamide is dispersed in the applesauce by admixing one to four (preferably four) 60 mg solid tablets of apalutamide in between about 50 g and about 200 g yogurt.

In another embodiment, the present invention is directed to a method for the preparation of a pharmaceutical composition comprising apalutamide dispersed in applesauce, wherein the apalutamide is dispersed in the applesauce by admixing one to four (preferably four) 60 mg solid tablets of apalutamide in between about 50 mL and about 200 mL fruit juice, preferably orange juice.

In another embodiment, the present invention is directed to a method for the preparation of a pharmaceutical composition comprising apalutamide dispersed in applesauce, wherein the apalutamide is dispersed in the applesauce by admixing one to four (preferably four) 60 mg solid tablets of apalutamide in between about 50 mL and about 200 mL tea, preferably green tea.

In another embodiment, the present invention is directed to a pharmaceutical composition comprising apalutamide dispersed in applesauce, wherein when administered to a male, exhibits a measured Cmax at median Tmax is achieved at between about 2 and about 3 hours. In another embodiment, the present invention is directed to a pharmaceutical composition comprising apalutamide dispersed in applesauce, wherein when administered to a male, exhibits a measured Cmax of about 2.4 μg/mL. In another embodiment, the present invention is directed to a pharmaceutical composition comprising apalutamide dispersed in applesauce, wherein when administered to a male, exhibits a measured AUC of about 225 μg*h/mL.

In some embodiments, methods of treating non-metastatic castration-resistant prostate cancer comprise, consist or, or consist essentially of administering a safe and effective amount of apalutamide to a male human with a non-metastatic castration-resistant prostate cancer, wherein the apalutamide is administered orally. In some embodiments, the apalutamide is administered daily. In some embodiments, the apalutamide is administered orally on a continuous daily dosage schedule. In further embodiments, the apalutamide is administered orally at a dose of about 240 mg per day. In other embodiments, the apalutamide is administered orally at a dose of about 60 mg four times per day.

In some embodiments, the apalutamide is present in a solid oral dosage form, for example as described in HESTER et al., US Patent Application Publication Number 2017/0360754 A1, published Dec. 21, 2017, VERRECK, G., US Patent Application Publication Number 2017/0360707 A1, published Dec. 21, 2017, and VERRECK, G., US Patent Application Publication Number 2017/0360713 A1, published Dec. 21, 2017, which are hereby incorporated by reference in their entireties. In some embodiments, the apalutamide is formulated as a tablet. In some embodiments, the apalutamide is formulated as a soft gel. In some embodiments, the apalutamide is formulated as a hard shell capsule.

In some embodiments, the enzalutamide is present in a solid oral dosage form, for example as described in LORENZ et al., US Patent Application Publication Number 2014/0100256 A1, published Apr. 10, 2014, which is incorporated herein in its entirety. In some embodiments, the enzalutamide is formulated as a tablet. In some embodiments, the enzalutamide is formulated as a soft gel. In some embodiments, the enzalutamide is formulated as a hard shell capsule.

In some embodiments, the darolutamide is present in a solid oral dosage form. In some embodiments, the darolutamide is formulated as a tablet. In some embodiments, the darolutamide is formulated as a soft gel. In some embodiments, the darolutamide is formulated as a tablet or a hard shell capsule.

Also provided herein are methods of treating prostate cancer including castrate resistant prostate cancer, metastatic castration resistant prostate cancer, metastatic castration sensitive prostate cancer, and non-metastatic castration resistant prostate cancer comprising, consisting of, or consisting essentially of administering an anti-androgen comprising apalutamide to a male human with a non-metastatic castration-resistant prostate cancer, wherein the apalutamide is administered in a mixture with applesauce; and wherein the dose of apalutamide is reduced when co-administered with one or more of:

(a) a CYP2C8 inhibitor, preferably gemfibrozil or clopidogrel; or

(b) a CYP3A4 inhibitor, preferably ketoconazole or ritonavir.

In some embodiments, the apalutamide is not co-administered with:

(a) medications that are primarily metabolized by CYP3A4, preferably darunavir, felodipine, midazolam or simvastatin;

(b) medications that are primarily metabolized by CYP2C19, preferably diazepam or omeprazole;

(c) medications that are primarily metabolized by CYP2C9, preferably warfarin or phenytoin; or

(d) medications that are substrates of UGT, preferably levothyroxine or valproic acid.

In further embodiments, the apalutamide is not co-administered with:

(a) medications that are P-gp substrates, preferably fexofenadine, colchicine, dabigatranetexilate or digoxin; or

(b) BCRP/OATP1B1 substrates, preferably lapatinib, methotrexate, rosuvastatin, or repaglinide.

In certain embodiments, the present invention is directed to methods of treatment as described herein, comprising the steps of
(a) mixing (for example, dispersing) apalutamide in a suitable vehicle (wherein the apalutamide is mixed in the suitable vehicle as described in any of the Examples which follow hereinafter); to yield an apalutamide-vehicle mixture (e.g. dispersion); and
(b) administering the apalutamide-vehicle mixture to a patient in need thereof; wherein the apalutamide-vehicle mixture is administered orally; and wherein the apalutamide-vehicle mixture is administered to the patient within about 6 hours (preferably within about 1 hour, more preferably within about 15 min, more preferably within about 5 min) of preparation of the apalutamide-vehicle mixture.

In certain embodiments of the present invention, the apalutamide-vehicle mixture is administered to the patient (i.e. is ingested by the patient) within about 6 hours of the preparation of the apalutamide-vehicle mixture (for example, within about 5 min, within about 10 min, within about 15 min, within about 30 min, within about 45 min, within about 1 hour, within about 2 hours, within about 3 hours, within about 4 hours, within about 5 hours, within about 6 hours or within any period of time between about 0 minutes and about 6 hours). Preferably, the apalutamide-vehicle mixture is administered to the patient within about 3 hours, more preferably within 1 hour, more preferably within about 30 min, more preferably within about 10 min, more preferably within about 5 min.

In certain embodiments, the present invention is directed to methods of treatment as described herein, comprising administering apalutamide to a patient in need thereof; wherein the apalutamide is administered as a mixture (e.g. dispersion) in applesauce; and wherein the administration comprises the steps of
1. placing one to four whole tablets of apalutamide (e.g. one, two, three or four whole, 60 mg tablets of apalutamide) in 4 ounces (120 mL) of applesauce and stirring to form a first mixture; wherein the apalutamide whole tablets are admixed with the applesauce in a suitable container;
2. holding the first mixture (of Step 1) without stirring for 15 minutes; and then stirring the first mixture; to yield a second mixture;
3. holding the second mixture (of Step 2) without stirring for an additional 15 minutes, and then stirring the second mixture until the tablets are mixed well with no chunks remaining; to yield an apalutamide-applesauce mixture (e.g. an apalutamide-applesauce dispersion);
4. swallowing the apalutamide-applesauce mixture (of Step 3) (for example by using a spoon or other utensil); wherein the apalutamide-applesauce mixture (of Step 3) is preferably swallowed within about one hour of preparation;
5. rinsing the container (which held the apalutamide-applesauce mixture) with 2 ounces (60 mL) of water and immediately drinking the contents; to yield a once rinsed container;
6. rinsing the once rinsed container (of Step 5) with 2 ounces (60 mL) of water and immediately drinking the contents.

One skilled in the art will recognize that in the methods of treatment and described above, a patient in need of treatment will swallow the apalutamide-applesauce mixture of Step 3 and the water rinses of Steps 5 and 6. However, Step 1-3 (preparation of the apalutamide-applesauce mixture) and the container rinsing portions of Steps 5 and 6 may be completed by either the patient or a caregiver.

In certain embodiments, the present invention is directed to pharmaceutical compositions comprising a mixture (e.g. a dispersion) of apalutamide in a suitable vehicle. In certain embodiments of the present invention, the pharmaceutical composition comprises a mixture (e.g. a dispersion) of apalutamide in applesauce. In certain embodiments of the present invention, the pharmaceutical composition comprises a mixture (e.g. a dispersion) of apalutamide in tea, preferably green tea. In certain embodiments of the present invention, the pharmaceutical composition comprises a mixture (e.g. a dispersion) of apalutamide in fruit juice, preferably orange juice. In certain embodiments of the present invention, the pharmaceutical composition comprises a mixture (e.g. a dispersion) of apalutamide in yogurt.

In certain embodiments, the present invention is directed to a method for the preparation of a pharmaceutical composition; wherein the pharmaceutical composition is an apalutamide-suitable vehicle mixture (e.g. a dispersion), as described in any of the Examples which follow hereinafter.

In certain embodiments, the present invention is directed to a method for the preparation of a pharmaceutical composition, comprising the steps of
1. placing one to four whole tablets of apalutamide (e.g. one, two, three or four whole, 60 mg tablets of apalutamide) in 4 ounces (120 mL) of applesauce and stirring to form a first mixture; wherein the apalutamide whole tablets are admixed with the applesauce in a suitable container;
2. holding the first mixture (of Step 1) without stirring for 15 minutes; and then stirring the first mixture; to yield a second mixture;
3. holding the second mixture (of Step 2) without stirring for an additional 15 minutes, and then stirring the second mixture until the tablets are mixed well with no chunks remaining; to yield an apalutamide-applesauce mixture (e.g. an apalutamide-applesauce dispersion);

In certain embodiments, the present invention is directed to a method for the preparation of a pharmaceutical composition; wherein the pharmaceutical composition is an apalutamide-suitable vehicle mixture (e.g. a dispersion); and wherein the method of preparation comprises the steps of
(a) in a container, adding one to four whole tablets of apalutamide (for example 60 mg whole tablets) into between about 50 ml and about 200 ml or between 50 g and 200 g of a suitable vehicle (preferably applesauce, a tea such as green tea, a fruit juice such as orange juice or yogurt); to yield a first mixture;
(b) mixing (preferably gently) the first mixture (for example with a spoon or spatula) for 10 secs; to yield a second mixture;
(c) holding the second mixture without mixing for 15 min; to yield a third mixture;
(d) mixing (preferably gently) the third mixture (for example with a spoon or spatula) for 10 secs; to yield a fourth mixture;
(e) repeating Steps (c) and (d) until a visually uniform mixture is formed.

In certain embodiments, the present invention is directed to a method for the preparation of a pharmaceutical composition; wherein the pharmaceutical composition comprises a mixture (e.g. dispersion) of apalutamide in applesauce (an apalutamide-applesauce dispersion); and wherein the method of preparation comprises the steps of
(f) in a container, adding one to four whole tablets of apalutamide (for example 60 mg whole tablets) into between about 50 ml and about 200 ml or between about 50 g and about 200 g of applesauce; to yield a first mixture;
(g) mixing (preferably gently) the first mixture (for example with a spoon or spatula) for 10 secs; to yield a second mixture;
(h) holding the second mixture without mixing for 15 min; to yield a third mixture;
(i) mixing (preferably gently) the third mixture (for example with a spoon or spatula) for 10 secs; to yield a fourth mixture;
(j) repeating Steps (c) and (d) until a visually uniform mixture is formed.

One skilled in the art will recognize that in the methods for the preparation of the pharmaceutical compositions described above, wherein the pharmaceutical compositions comprise a mixture (e.g. dispersion) of apalutamide in a suitable vehicle such as applesauce, tea such as green tea, fruit juice such as orange juice or yogurt; the visually uniform mixture prepared at the end is the preparation steps is a mixture (e.g. dispersion) of apalutamide in the corresponding suitable vehicle.

In certain embodiments, the present invention is directed to any one or more methods of treatment as described herein, wherein enzalutamide is substituted for apalutamide, at one or more suitable selected dosage amounts. In certain embodiments, the present invention is directed to any one or more methods of treatment as described herein, wherein darolutamide is substituted for apalutamide, at one or more suitable selected dosage amounts.

In certain embodiments, the present invention is directed to any one or more pharmaceutical compositions as described herein, wherein enzalutamide is substituted for apalutamide, at one or more suitable selected dosage amounts. In certain embodiments, the present invention is directed to any one or more pharmaceutical compositions of treatment as described herein, wherein darolutamide is substituted for apalutamide, at one or more suitable selected dosage amounts.

In an embodiment, the present invention is directed to a method for the preparation of a pharmaceutical composition comprising mixing or admixing apalutamide in a suitable vehicle, as described herein. In another embodiment, the present invention is directed to a method for the preparation of a pharmaceutical composition comprising mixing or admixing apalutamide in a suitable vehicle, as described herein.

In an embodiment, the present invention is directed to a method for the preparation of a pharmaceutical composition comprising admixing an anti-androgen (preferably apalutamide, enzalutamide or doralutamide, more preferably apalutamide) in applesauce, as described herein. In another embodiment, the present invention is directed to a method for the preparation of a pharmaceutical composition comprising admixing apalutamide, enzalutamide or doralutamide, in applesauce, as described herein. In a further embodiment, the present invention is directed to a method for the preparation of a pharmaceutical composition comprising admixing apalutamide, enzalutamide in applesauce, as described herein. In an embodiment, the present invention is directed to a method for the preparation of a pharmaceutical composition comprising admixing enzalutamide in applesauce, as described herein. In an embodiment, the present invention is directed to a method for the preparation of a pharmaceutical composition comprising admixing doralutamide in applesauce, as described herein.

In certain embodiments, the present invention is directed to any method for the preparation of a pharmaceutical composition as described herein, wherein enzalutamide is substituted for apalutamide, at one or more suitable selected dosage amounts. In certain embodiments, the present invention is directed to any method for the preparation of a pharmaceutical composition of treatment as described herein, wherein darolutamide is substituted for apalutamide, at one or more suitable selected dosage amounts.

### BRIEF DESCRIPTION OF THE FIGURES

FIG. 1 illustrates the Time and Events Schedule for the clinical trial described in Example 1.

FIG. 2 illustrates the two page taste questionnaire administered to trial participants.

FIG. 3 illustrates subject disposition for the clinical trial described in Example 1.

FIG. 4 illustrates a tabulated summary of demographics and baseline characteristics; for the safety analysis set of patients.

FIG. 5 illustrates a tabulated listed of the randomization codes for participants in the trial of Example 1.

FIG. 6 illustrates a tabulated listing of the individual plasma concentrations of apalutamide, including descriptive statistics by treatment for Treatment A: 240 mg Apalutamide Tablets swallowed whole.

FIG. 7 illustrates a tabulated listing of the individual plasma concentrations of apalutamide, including descriptive statistics by treatment for Treatment B: 240 mg Apalutamide Tablets as a dispersed mixture in applesauce.

FIG. 8 illustrated a tabulated listing of the mean plasma concentration-time profiles for Apalutamide for Treatment A and Treatment B, applying (A) linear and (B) semi-log scales, respectively.

FIG. 9 illustrates a tabulated listing of the individual PK parameters of Apalutamide, including descriptive statistics for Treatment A: 240 mg Apalutamide Tablets swallowed whole.

FIG. 10 illustrates a tabulated listing of the individual PK parameters of Apalutamide, including descriptive statistics for Treatment B: 240 mg Apalutamide Tablets as a dispersed mixture in applesauce.

FIG. 11 illustrates a tabulated listing of the individual Apalutamide PK parameter treatment ratios, including descriptive statistics.

FIG. 12 illustrates a tabulated listing of treatment-emergent adverse events (TEAEs) by system organ class and preferred term, by treatment group; safety analysis group.

FIG. 13 illustrates a tabulated listing of treatment-emergent adverse events (TEAEs) by system organ class and preferred term, total both treatment groups, safety analysis group.

FIG. 14 illustrates a tabulated listing of the summary results of the taste questionnaire, safety analysis set for both treatment groups.

FIG. 15 is a Kaplan-Meier Plot of Overall Survival (OS); Intent-to-treat mCSPC Population (TITAN).

FIG. 16 is a Kaplan-Meier Plot of Radiographic Progression-Free Survival (rPFS); Intent-to-treat mCSPC Population (TITAN).

FIG. 17 is a Kaplan-Meier Metastasis-Free Survival (MFS) Curve in SPARTAN (nmCRPC).

### DETAILED DESCRIPTION OF THE INVENTION

The present invention is directed to methods of treating non-metastatic castration-resistant prostate cancer comprising, consisting or consisting essentially of administer a safe and effective amount of apalutamide to a male human with a non-metastatic castration-resistant prostate cancer.

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of” and “consisting essentially of.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Androgen receptor (AR) is a member of the steroid and nuclear receptor superfamily Among this large family of proteins, only five vertebrate steroid receptors are known and include the androgen receptor, estrogen receptor, progesterone receptor, glucocorticoid receptor, and mineralocorticoid receptor. AR is a soluble protein that functions as an intracellular transcriptional factor. AR function is regulated by the binding of androgens, which initiates sequential conformational changes of the receptor that affect receptor-protein interactions and receptor-DNA interactions.

AR is mainly expressed in androgen target tissues, such as the prostate, skeletal muscle, liver, and central nervous system (CNS), with the highest expression level observed in the prostate, adrenal gland, and epididymis. AR can be activated by the binding of endogenous androgens, including testosterone and 5-dihydrotestosterone (5a-DHT).

The androgen receptor (AR), located on Xql 1-12, is a 110 kD nuclear receptor that, upon activation by androgens, mediates transcription of target genes that modulate growth and differentiation of prostate epithelial cells. Similar to the other steroid receptors, unbound AR is mainly located in the cytoplasm and associated with a complex of heat shock proteins (HSPs) through interactions with the ligand-binding domain Upon agonist binding, AR goes through a series of conformational changes: the heat shock proteins dissociate from AR, and the transformed AR undergoes dimerization, phosphorylation, and translocation to the nucleus, which is mediated by the nuclear localization signal. Translocated receptor then binds to the androgen response element (ARE), which is characterized by the six-nucleotide half-site consensus sequence 5′-TGTTCT-3′ spaced by three random nucleotides and is located in the promoter or enhancer region of AR gene targets. Recruitment of other transcription co-regulators (including co-activators and co-repressors) and transcriptional machinery further ensures the transactivation of AR-regulated gene expression. All of these processes are initiated by the ligand-induced conformational changes in the ligand-binding domain.

AR signaling is crucial for the development and maintenance of male reproductive organs including the prostate gland, as genetic males harboring loss of function AR mutations and mice engineered with AR defects do not develop prostates or prostate cancer. This dependence of prostate cells on AR signaling continues even upon neoplastic transformation. Androgen depletion (such as using GnRH agonists) continues to be the mainstay of prostate cancer treatment. However, androgen depletion is usually effective for a limited duration and prostate cancer evolves to regain the ability to grow despite low levels of circulating androgens.

Castration resistant prostate cancer (CRPC) is a lethal phenotype and almost all of patients will die from prostate cancer. Interestingly, while a small minority of CRPC does bypass the requirement for AR signaling, the vast majority of CRPC, though frequently termed “androgen independent prostate cancer” or “hormone refractory prostate cancer,” retains its lineage dependence on AR signaling.

Prostate cancer is the second most common cause of cancer death in men in the US, and approximately one in every six American men will be diagnosed with the disease during his lifetime. Treatment aimed at eradicating the tumor is unsuccessful in 30% of men, who develop recurrent disease that is usually manifest first as a rise in plasma prostate-specific antigen (PSA) followed by spread to distant sites. Given that prostate cancer cells depend on androgen receptor (AR) for their proliferation and survival, these men are treated with agents that block production of testosterone (e.g., GnRH agonists), alone or in combination with anti-androgens (e.g., bicalutamide), which antagonize the effect of any residual testosterone on AR. The approach is effective as evidenced by a drop in PSA and regression of visible tumor (if present) in some patients; however, this is followed by regrowth as a castration resistant prostate cancer (CRPC) to which most patients eventually succumb. Recent studies on the molecular basis of CRPC have demonstrated that CRPC continues to depend on AR signaling and that a key mechanism of acquired resistance is an elevated level of AR protein (Nat. Med, 2004, 10, 33-39). AR targeting agents with activity in castration sensitive and castration resistant prostate cancer have great promise in treating this lethal disease.

The course of prostate cancer from diagnosis to death is best categorized as a series of clinical states based on the extent of disease, hormonal status, and absence or presence of detectable metastases: localized disease, rising levels of prostate-specific antigen (PSA) after radiation therapy or surgery with no detectable metastases, and clinical metastases in the non-castrate or castrate state. Although surgery, radiation, or a combination of both can be curative for patients with localized disease, a significant proportion of these patients have recurrent disease as evidenced by a rising level of PSA, which can lead to the development of metastases, especially in the high risk group—a transition to the lethal phenotype of the disease.

Androgen depletion is the standard treatment with a generally predictable outcome: decline in PSA, a period of stability in which the tumor does not proliferate, followed by rising PSA and regrowth as castration-resistant disease. Molecular profiling studies of castration-resistance prostate cancers commonly show increased androgen receptor (AR) expression, which can occur through AR gene amplification or other mechanisms.

Anti-androgens are useful for the treatment of prostate cancer during its early stages. However, prostate cancer often advances to a ‘hormone-refractory’ state in which the disease progresses in the presence of continued androgen ablation or anti-androgen therapy. Instances of antiandrogen withdrawal syndrome have also been reported after prolonged treatment with anti-androgens. Antiandrogen withdrawal syndrome is commonly observed clinically and is defined in terms of the tumor regression or symptomatic relief observed upon cessation of antiandrogen therapy. AR mutations that result in receptor promiscuity and the ability of these anti-androgens to exhibit agonist activity might at least partially account for this phenomenon. For example, hydroxyflutamide and bicalutamide act as AR agonists in T877A and W741L/W741C AR mutants, respectively.

In the setting of prostate cancer cells that were rendered castration resistant via overexpression of AR, it has been demonstrated that certain anti-androgen compounds, such as bicalutamide, have a mixed antagonist/agonist profile (Science, 2009 May 8; 324(5928): 787-90). This agonist activity helps to explain a clinical observation, called the anti-androgen withdrawal syndrome, whereby about 30% of men who progress on AR antagonists experience a decrease in serum PSA when therapy is discontinued (J Clin Oncol, 1993. 11(8): p. 1566-72).

### Prostate Cancer Stages

In the early stages of prostate cancer, the cancer is localized to the prostate. In these early stages, treatment typically involves either surgical removal of the prostate or radiation therapy to the prostate or observation only with no active intervention therapy in some patients. In the early stages where the prostate cancer is localized and requires intervention, surgery or radiation therapy are curative by eradicating the cancerous cells. About 30% of the time these procedures fail, and the prostate cancer continues to progress, as typically evidenced by a rising PSA level. Men whose prostate cancer has progressed following these early treatment strategies are said to have advanced or recurrent prostate cancer.

Because prostate cancer cells depend on the androgen receptor (AR) for their proliferation and survival, men with advanced prostate cancer are treated with agents that block the production of testosterone (e.g., GnRH agonists), alone or in combination with anti-androgens (e.g., bicalutamide), which antagonize the effect of any residual testosterone on AR. These treatments reduce serum testosterone to castrate levels, which generally slows disease progression for a period of time. The approach is effective as evidenced by a drop in PSA and the regression of visible tumors in some patients. Eventually, however, this is followed by regrowth referred to as castration-resistant prostate cancer (CRPC), to which most patients eventually succumb.

Castration-resistant prostate cancer (CRPC) is categorized as non-metastatic or metastatic, depending on whether or not the prostate cancer has metastasized to other parts of the body.

In some embodiments, prior to treatment with a second-generation anti-androgen men with non-metastatic CRPC are characterized as having the following:

1. Histologically or cytologically confirmed adenocarcinoma of the prostate without neuroendocrine differentiation or small cell features, with high risk for development of metastases.

2. Castration-resistant prostate cancer demonstrated during continuous androgen deprivation therapy (ADT)/post orchiectomy. For example defined as 3 consecutive rises of PSA, 1 week apart, resulting in two 50% increases over the nadir, with the last PSA>2 ng/mL.

3. Maintain castrate levels of testosterone (<50 ng/dL [1.72 nmol/L]) within 4 weeks of randomization and throughout the study.

4. Absence of distant metastasis by bone scan, CT or MRI scans.

### Anti-Androgens

As used herein, the term “anti-androgen” refers to a group of hormone receptor antagonist compounds that are capable of preventing or inhibiting the biologic effects of androgens on normally responsive tissues in the body. In some embodiments, an anti-androgen is a small molecule. In some embodiments, an anti-androgen is an AR antagonist. In some embodiments, an anti-androgen is an AR full antagonist. In some embodiments, an anti-androgen is a first-generation anti-androgen. In some embodiments, an anti-androgen is a second-generation anti-androgen.

As used herein, the term “AR antagonist” or “AR inhibitor” are used interchangeably herein and refer to an agent that inhibits or reduces at least one activity of an AR polypeptide. Exemplary AR activities include, but are not limited to, co-activator binding, DNA binding, ligand binding, or nuclear translocation.

As used herein, a “full antagonist” refers to an antagonist, which, at an effective concentration, essentially completely inhibits an activity of an AR polypeptide. As used herein, a “partial antagonist” refers an antagonist that is capable of partially inhibiting an activity of an AR polypeptide, but that, even at a highest concentration is not a full antagonist. By ‘essentially completely’ is meant at least about 80%, at least about 90%, at least about 95%, at least about 96%, at least about 97%, at least about 98% at least about 99%, or greater inhibition of the activity of an AR polypeptide.

As used herein, the term “first-generation anti-androgen” refers to an agent that exhibits antagonist activity against a wild-type AR polypeptide. However, first-generation anti-androgens differ from second-generation anti-androgens in that first-generation anti-androgens can potentially act as agonists in castration resistant prostate cancers (CRPC). Exemplary first-generation anti-androgens include, but are not limited to, flutamide, nilutamide and bicalutamide.

As used herein, the term “second-generation anti-androgen” refers to an agent that exhibits full antagonist activity against a wild-type AR polypeptide. Second-generation anti-androgens differ from first-generation anti-androgens in that second-generation anti-androgens act as full antagonists in cells expressing elevated levels of AR, such as for example, in castration resistant prostate cancers (CRPC). Exemplary second-generation anti-androgens include 4-[7-(6-cyano-5-trifluoromethylpyridin-3-yl)-8-oxo-6-thioxo-5,7-diazaspiro[3.4]oct-5-yl]-2-fluoro-N-methylbenzamide (also known as apalutamide or ARN-509; CAS No. 956104-40-8); 4-(3-(4-cyano-3-(trifluoromethyl)phenyl)-5,5-dimethyl-4-oxo-2-thioxoimidazolidin-1-yl)-2-fluoro-N-methylbenzamide (also known as MDV3100 or enzalutamide; CAS No: 915087-33-1) and RD162 (CAS No. 915087-27-3). In some embodiments, a second-generation anti-androgen binds to an AR polypeptide at or near the ligand binding site of the AR polypeptide.

[chemical structure]

4-[7-(6-cyano-5-trifluoromethylpyridin-3-yl)-8-oxo-6-thioxo-5,7-diazaspiro[3.4]oct-5-yl]-2-fluoro-N-methylbenzamide (apalutamide)

[chemical structure]

### 4-(3-(4-cyano-3-(trifluoromethyl)phenyl)-5,5-dimethyl-4-oxo-2-thioxoimidazolidin-1-yl)-2-fluoro-N-methylbenzamide (enzalutamide)

In some embodiments, an anti-androgen contemplated in the methods described herein inhibits AR nuclear translocation, such as darolutamide, DNA binding to androgen response elements, and coactivator recruitment. In some embodiments, an anti-androgen contemplated in the methods described herein exhibits no agonist activity in AR-overexpressing prostate cancer cells.

Apalutamide is a second-generation anti-androgen that binds directly to the ligand-binding domain of AR, impairing nuclear translocation, AR binding to DNA and AR target gene modulation, thereby inhibiting tumor growth and promoting apoptosis. Apalutamide binds AR with greater affinity than bicalutamide, and induces partial or complete tumor regression in non-castrate hormone-sensitive and bicalutamide-resistant human prostate cancer xenograft models (Clegg et al. Cancer Res. Mar. 15, 2012 72; 1494). Apalutamide lacks the partial agonist activity seen with bicalutamide in the context of AR overexpression.

Darolutamide, BAY1841788 or ODM-201, is an AR antagonist that includes two diastereomers—ORM-16497 and ORM-16555. It has activity against known AR mutants that confer resistance to other second-generation antiandrogens. Darolutamide binds to the AR with high affinity, and impairs subsequent androgen-induced nuclear translocation of AR and transcription of AR gene target. Matsubara, N Mukal, H., Hosono, A. et al. Cancer Chemother Pharmacci (2017) 80: 1063.

In one aspect described herein are methods of prostate cancer including castrate resistant prostate cancer, metastatic castration resistant prostate cancer, metastatic castration sensitive prostate cancer and non-metastatic castration resistant prostate cancer comprising, consisting of, or consisting essentially of administering a safe and effective amount of an anti-androgen to a male human with a prostate cancer including castrate resistant prostate cancer, metastatic castration resistant prostate cancer, metastatic castration sensitive prostate cancer, and non-metastatic castration resistant prostate cancer. In another aspect described herein are methods of treating a male human having prostate cancer including castrate resistant prostate cancer, metastatic castration resistant prostate cancer, metastatic castration sensitive prostate cancer and non-metastatic castration resistant prostate cancer comprising, consisting of, or consisting essentially of administering a safe and effective amount of an anti-androgen to a male human with a prostate cancer including castrate resistant prostate cancer, metastatic castration resistant prostate cancer, metastatic castration sensitive prostate cancer, and non-metastatic castration resistant prostate cancer. In the following disclosure, “methods of treating non-metastatic castration-resistant prostate cancer,” may alternatively be recited as “methods of treating a male human having non-metastatic castration-resistant prostate cancer.” For the sake of brevity, each possible alternative is not parsed out.

In a Phase II clinical trial of male humans with high risk non-metastatic CRPC, treatment-naive metastatic CRPC and metastatic CRPC that progressed after prior treatment with abiraterone acetate (ZYTIGA) plus prednisone, oral administration of 240 mg of apalutamide on a continuous daily dosing schedule was very well tolerated and resulted in robust and durable PSA responses, as well as evidence of objective responses. A total of 25 patients with chemotherapy and abiraterone acetate-plus prednisone naive metastatic CRPC who had progressed on standard androgen deprivation therapy (treatment-naive (TN) cohort) and 21 patients who progressed after treatment with abiraterone acetate plus prednisone (PA cohort) were orally administered 240 mg of apalutamide on a continuous daily dosing schedule. The primary objective was to assess antitumor activity and PSA kinetics as defined by the Prostate Cancer Clinical Trials Working Group (PCWG2) criteria. Preliminary results demonstrated 12-week PSA declines of >50% or more from baseline in 88% and 29% of the TN and PA cohorts, respectively. The median time to PSA progression was not reached for the TN cohort during the preliminary 12-week period, and was 16 weeks in the PA cohort. In addition, the objective response rate (by RECIST) was 63%> in the TN patients presenting with measurable disease at baseline, further confirming the antitumor activity of apalutamide.

A total of 47 patients with non-metastatic CRPC were orally administered 240 mg of apalutamide on a continuous daily dosing schedule. At 12 weeks of treatment, 91% of the patients had a >50% decline in PSA as compared to baseline. At 24 weeks, the percentage of patients who had >50% decline in PSA remained at 91% and the percentage of patients who had >90%>decline in PSA was 55%>, confirming the durability of response to apalutamide. The median time to PSA progression was not reached in this observed time period.

### Certain Terminology

The term “cancer” as used herein refers to an abnormal growth of cells which tend to proliferate in an uncontrolled way and, in some cases, to metastasize (spread).

The term “prostate cancer” as used herein refers to histologically or cytologically confirmed adenocarcinoma of the prostate.

The term “androgen-deprivation therapy (ADT)” refers to the reduction of androgen levels in a prostate cancer patient to castrated levels of testosterone (<50 ng/dL). Such treatments can include orchiectomy or the use of gonadotropin-releasing hormone agonists or antagonists. ADT includes surgical castration (orchiectomy) and/or the administration of luteinizing hormone-releasing hormone (“LHRH”) agonists to a human. Examples of LHRH agonists include goserelin acetate, histrelin acetate, leuprolide acetate, and triptorelin palmoate. Physicians can prescribe LHRH agonists in accordance with instructions, recommendations and practices. This may include about 0.01 mg to about 20 mg of goserelin over a period of about 28 days to about 3 months, preferably about 3.6 mg to about 10.8 mg of goserelin over a period of about 28 days to about 3 months; about 0.01 mg to about 200 mg of leuprolide over a period of about 3 days to about 12 months, preferably about 3.6 mg of leuprolide over a period of about 3 days to about 12 months; or about 0.01 mg to about 20 mg of triptorelin over a period of about 1 month, preferably about 3.75 mg of triptorelin over a period of 1 month. About 50 mg of histrelin acetate over a period of 12 months of histrelin acetate or about 50 μg per day of histrelin acetate.

The term “locally advanced prostate cancer” refers to prostate cancer where all actively cancerous cells appear to be confined to the prostate and the associated organs or neighbor organs (e.g., seminal vesicle, bladder neck, and rectal wall).

The term “high-risk localized prostate cancer” refers to locally advanced prostate cancer that has a probability of developing metastases or recurrent disease after primary therapy with curative intent. In some embodiments, high risk for development of metastases is defined as prostate specific antigen doubling time (PSADT)<20 months, <19 months, <18 months, <17 months, <16 months, <15 months, <14 months, <13 months, <12 months, or <11 months, <10 months, <9 months, <8 months, <7 months, <6 months, <5 months, <4 months, <3 months, <2 months, or <1 month. In some embodiments, high risk for development of metastases is defined as prostate specific antigen doubling time (PSADT)<10 months. In some embodiments, high risk for development of metastases is defined as having a high Gleason score or bulky tumor.

The term “castration-sensitive prostate cancer” refers to cancer that is responsive to androgen-deprivation therapy (ADT) either as localized disease, biochemical relapse or in the metastatic setting.

The term “metastatic castration-sensitive prostate cancer” refers to cancer that has spread (metastasized) to other areas of the body, e.g., the bone, lymph nodes or other parts of the body in a male, and that is responsive to androgen-deprivation therapy (ADT).

The term “non-metastatic castration-sensitive prostate cancer” refers to cancer that has not spread (metastasized) in a male, and that is responsive to androgen-deprivation therapy (ADT). In some embodiments, non-metastatic castration-sensitive prostate cancer is assessed with bone scan and computed tomography (CT) or magnetic resonance imaging (MRI) scans.

The term “CRPC” as used herein refers to castration-resistant prostate cancer. CRPC is prostate cancer that continues to grow despite the suppression of male hormones that fuel the growth of prostate cancer cells.

The term “metastatic castration-resistant prostate cancer” refers to castration-resistant prostate cancer that has metastasized to other parts of the human body.

The term “NM-CRPC” as used herein refers to non-metastatic castration-resistant prostate cancer. In some embodiments, NM-CRPC is assessed with bone scan and computed tomography (CT) or magnetic resonance imaging (MRI) scans.

The term “chemotherapy naive metastatic castration-resistant prostate cancer” refers to metastatic castration-resistant prostate cancer that has not been previously treated with a chemotherapeutic agent.

The term “post-abiraterone acetate-prednisone treated metastatic castration-resistant prostate cancer” refers to metastatic castration-resistant prostate cancer that has already been treated with abiraterone acetate.

In some embodiments, the non-metastatic castration-resistant prostate cancer is a high risk non-metastatic castration-resistant prostate cancer. The term “high risk NM-CRPC” refers to probability of a man with NM-CRPC developing metastases. In some embodiments, high risk for development of metastases is defined as prostate specific antigen doubling time (PSADT)<20 months, <19 months, <18 months, <17 months, <16 months, <15 months, <14 months, <13 months, <12 months, or <11 months, <10 months, <9 months, <8 months, <7 months, <6 months, <5 months, <4 months, <3 months, <2 months, or <1 month. In some embodiments, high risk for development of metastases is defined as prostate specific antigen doubling time (PSADT)<10 months. In some embodiments, high risk for development of metastases is defined as having local-regional recurrence (e.g. primary tumor bed, bladder neck, anastomotic area, pelvic lymph nodes).

The terms “co-administration” or the like, as used herein, encompass administration of the selected therapeutic agents to a single patient, and are intended to include treatment regimens in which the agents are administered by the same or different route of administration or at the same or different time.

The term “pharmaceutical combination” as used herein, means a product that results from the mixing or combining of more than one active ingredient and includes both fixed and non-fixed combinations of the active ingredients. The term “fixed combination” means that the active ingredients, e.g., apalutamide and a co-agent, are both administered to a patient simultaneously in the form of a single unit or single dosage form. The term “non-fixed combination” means that the active ingredients, e.g., apalutamide and a co-agent, are administered to a patient as separate units or separate dosage forms, either simultaneously, concurrently or sequentially with no specific intervening time limits, wherein such administration provides safe and effective levels of the two active ingredients in the body of the human male. The latter also applies to cocktail therapy, e.g., the administration of three or more active ingredients.

The term “FDHT-PET” refers to 18F-16P-fluoro-5a-dihydrotestosterone Positron Emission Tomography and is a technique that uses a tracer based on dihydrotestosterone, and allows for a visual assessment of ligand binding to the androgen receptor in a patient. It may be used to evaluate pharmacodynamics of an androgen receptor directed therapy [0099] The term “continuous daily dosing schedule” refers to the administration of a particular therapeutic agent without any drug holidays from the particular therapeutic agent. In some embodiments, a continuous daily dosing schedule of a particular therapeutic agent comprises administration of a particular therapeutic agent every day at roughly the same time each day.

The terms “treat” and “treatment” refer to the treatment of a patient afflicted with a pathological condition and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “metastasis-free survival” or “MFS” refers to the percentage of subjects in a study who have survived without cancer spread for a defined period of time or death. MFS is usually reported as time from the beginning of enrollment, randomization or treatment in the study. MFS is reported for an individual or a study population. In the context of treatment of CRPC with an anti-androgen, an increase in the metastasis-free survival is the additional time that is observed without cancer having spread or death, whichever occurs first, as compared to treatment with placebo. In some embodiments, the increase in the metastasis-free survival is about 1 month, about 2 months, about 3 months, about 4 months, about 5 months, about 6 months, about 7 months, about 8 months, about 10 months, about 11 months, about 12 months, about 13 months, about 14 months, about 15 months, about 16 months, about 17 months, about 18 months, about 19 months, about 20 months, or greater than 20 months. In some embodiments, administration of a safe and effective amount of an anti-androgen provides an increase in the metastasis-free survival of a male human, optionally wherein the increase in the metastasis-free survival is relative to the mean survival rate of a population of male humans with the non-metastatic castration-resistant prostate cancer, said population having been treated with a placebo. In some embodiments, metastasis-free survival refers to the time from randomization to the time of first evidence of BICR-confirmed bone or soft tissue distant metastasis or death due to any cause, whichever occurs first.

The term “time to metastasis” is the time from randomization to the time of the scan that shows first evidence of BICR-confirmed radiographically detectable bone or soft tissue distant metastasis. In some embodiments, administration of a safe and effective amount of an anti-androgen provides improved anti-tumor activity as measured by time to metastasis (TTM).

The term “progression-free survival” is based on RECIST v1.1 and is defined as follows: For subjects with at least one measurable lesion, progressive disease is defined as at least a 20% increase in the sum of diameters of target lesions taking as reference the smallest sum on study (this includes the baseline sum if that is the smallest on study). In addition to the relative increase of 20%, the sum must also demonstrate an absolute increase of at least 5 mm. Furthermore, the appearance of one or more new lesions is also considered progression. For subjects with onlynon-measurable disease observed on CT or MRI scans, unequivocal progression (representative of overall disease status change) or the appearance of one or more new lesions was considered progression. For new bone lesions detected on bone scans, a second imaging modality (e.g., CT or MRI) was required to confirm progression. In some embodiments, administration of a safe and effective amount of an anti-androgen provides improved anti-tumor activity as measured by progression-free survival rate.

The term “time to symptomatic progression” is defined as the time from randomization to documentation in the CRF of any of the following (whichever occurs earlier): (1) development of a skeletal-related event (SRE): pathologic fracture, spinal cord compression, or need for surgical intervention or radiation therapy to the bone; (2) pain progression or worsening of disease-related symptoms requiring initiation of a new systemic anti-cancer therapy; or (3) development of clinically significant symptoms due to loco-regional tumor progression requiring surgical intervention or radiation therapy. In some embodiments, administration of a safe and effective amount of an anti-androgen provides improved anti-tumor activity as measured by time to symptomatic progression.

The term “overall survival” is defined as the time from randomization to the date of death due to any cause. Survival data for subjects who are alive at the time of the analysis was to be censored on the last known date that they were alive. In addition, for subjects with no post-baseline information survival, data was to be censored on the date of randomization; for subjects who are lost to follow-up or who withdraw consent, data is censored on the last known date that they were alive. In some embodiments, administration of a safe and effective amount of an anti-androgen provides improved anti-tumor activity as measured by overall survival.

The term “time to initiation of cytotoxic chemotherapy” is defined as the time from randomization to documentation of a new cytotoxic chemotherapy being administered to the subject (e.g., survival follow-up CRF). Time to initiation of cytotoxic chemotherapy for subjects who do not start a cytotoxic chemotherapy is censored on the date of last contact. In some embodiments, administration of a safe and effective amount of an anti-androgen provides improved anti-tumor activity as measured by time to cytotoxic chemotherapy.

The term “progression-free survival with the first subsequent therapy” (PFS2) is defined as the time from randomization to investigator-assessed disease progression (PSA, radiographic, symptomatic, or any combination) during first subsequent anti-cancer therapy or death (any cause) prior to the start of the second subsequent anti-cancer therapy, whichever occurs first. Progression data for subjects without documented progression after subsequent therapy is censored at the last date known to be progression-free or date of death. In some embodiments, administration of a safe and effective amount of an anti-androgen provides improved anti-tumor activity as measured progression-free survival with the first subsequent therapy.

Prostate specific antigen response and time to PSA progression is assessed at the time of the primary analysis of MFS according to the Prostate Cancer Working Group (PCWG2) criteria. The time to PSA progression is calculated as the time from randomization to the time when the criteria for PSA progression according to PCWG2 are met.

The term “placebo” as used herein means administration of a pharmaceutical composition that does not include a second-generation anti-androgen. In the context of treatment of CRPC, men that are administered an anti-androgen or placebo will need to continue to maintain castrated levels of testosterone by either co-administration of a GnRH agonist/antagonist or orchiectomy.

The term “survival benefit” as used herein means an increase in survival of the patient from time of randomization on the trial of administered drug to death. In some embodiments, the survival benefit is about 1, about 2, about 3, about 4, about 5, about 6, about 7, about 8, about 9, about 10, about 15, about 20, about 25, about 30, about 35, about 40, about 45, about 50, about 55, about 60, about 80, about 100 months or greater than 100 months.

The term “delay in symptoms related to disease progression” as used herein means an increase in time in the development of symptoms such as pain, urinary obstruction and quality of life considerations from the time of randomization on the trial of administered drug.

The term ‘randomization’ as it refers to a clinical trial refers to the time when the patient is confirmed eligible for the clinical trial and gets assigned to a treatment arm.

The terms “kit” and “article of manufacture” are used as synonyms.

The term “subject” and “patient” and “human” are used interchangeably.

The term, “drug product” or “approved drug product” is product that contains an active pharmaceutical ingredient that has been approved for marketing for at least one indication by a governmental authority, e.g., the Food and Drug Administration or the similar authority in other countries.

The term “Reference Listed Drug (RLD)” is a drug product to which new generic versions are compared to show that they are bioequivalent. 21 CFR 314.3(10) It is also a medicinal product that has been granted marketing authorization by a Member State of the European Union or by the Commission on the basis of a completed dossier, i.e., with the submission of quality, pre-clinical and clinical data in accordance with Articles 8(3), 10a, 10b or 10c of Directive 2001/83/EC and to which the application for marketing authorization for a generic/hybrid medicinal product refers, by demonstration of bioequivalence, usually through the submission of the appropriate bioavailability studies.

In the United States, a company seeking approval to market a generic equivalent must refer to the RLD in its Abbreviated New Drug Application (ANDA). For example, an ANDA applicant relies on the FDA's finding that a previously approved drug product, i.e., the RLD, is safe and effective, and must demonstrate, among other things, that the proposed generic drug product is the same as the RLD in certain ways. Specifically, with limited exceptions, a drug product for which an ANDA is submitted must have, among other things, the same active ingredient(s), conditions of use, route of administration, dosage form, strength, and (with certain permissible differences) labeling as the RLD. The RLD is the listed drug to which the ANDA applicant must show its proposed ANDA drug product is the same with respect to active ingredient(s), dosage form, route of administration, strength, labeling, and conditions of use, among other characteristics. In the electronic Orange Book, there will is a column for RLDs and a column for reference standards. In the printed version of the Orange Book, the RLDs and reference standards are identified by specific symbol. For an ANDA based on an approved suitability petition (a petitioned ANDA), the reference listed drug generally is the listed drug referenced in the approved suitability petition.

A reference standard is the drug product selected by FDA that an applicant seeking approval of an ANDA must use in conducting an in vivo bioequivalence study required for approval. FDA generally selects a single reference standard that ANDA applicants must use in bioequivalence testing. Ordinarily, FDA will select the reference listed drug as the reference standard. However, in some instances (e.g., where the reference listed drug has been withdrawn from sale and FDA has determined it was not withdrawn for reasons of safety or effectiveness, and FDA selects an ANDA as the reference standard), the reference listed drug and the reference standard may be different.

FDA identifies reference listed drugs in the Prescription Drug Product, OTC Drug Product. and Discontinued Drug Product Lists. Listed drugs identified as reference listed drugs represent drug products upon which an applicant can rely in seeking approval of an ANDA. FDA intends to update periodically the reference listed drugs identified in the Prescription Drug Product, OTC Drug Product, and Discontinued Drug Product Lists, as appropriate.

FDA also identifies reference standards in the Prescription Drug Product and OTC Drug Product Lists. Listed drugs identified as reference standards represent the FDA's best judgment at this time as to the appropriate comparator for purposes of conducting any in in vivo bioequivalence studies required for approval.

In some instances when FDA has not designated a listed drug as a reference listed drug, such listed drug may be shielded from generic competition. If FDA has not designated a reference listed drug for a drug product the applicant intends to duplicate, the potential applicant may ask FDA to designate a reference listed drug for that drug product.

FDA may, on its own initiative, select a new reference standard when doing so will help to ensure that applications for generic drugs may be submitted and evaluated, e.g., in the event that the listed drug currently selected as the reference standard has been withdrawn from sale for other than safety and efficacy reasons.

In Europe, Applicants identify in the application form for its generic/hybrid medicinal product, which is the same as a ANDA or sNDA drug product, the reference medicinal product (product name, strength, pharmaceutical form, MAH, first authorization, Member State/Community), which is synonymous with a RLD, as follows:

1. The medicinal product that is or has been authorized in the EEA, used as the basis for demonstrating that the data protection period defined in the European pharmaceutical legislation has expired. This reference medicinal product, identified for the purpose of calculating expiry of the period of data protection, may be for a different strength, pharmaceutical form, administration route or presentation than the generic/hybrid medicinal product.

2. The medicinal product, the dossier of which is cross-referred to in the generic/hybrid application (product name, strength, pharmaceutical form, MAH, marketing authorization number). This reference medicinal product may have been authorized through separate procedures and under a different name than the reference medicinal product identified for the purpose of calculating expiry of the period of data protection. The product information of this reference medicinal product will, in principle, serve as the basis for the product information claimed for the generic/hybrid medicinal product.

3. The medicinal product (product name, strength, pharmaceutical form, MAH, Member State of source) used for the bioequivalence study(ies) (where applicable).

The different abbreviated approval pathways for drug products under the FD&C Act—the abbreviated approval pathways described in section 505(j) and 505(b)(2) of the FD&C Act (21 U.S.C. 355(j) and 21 U.S.C. 23 355(b)(2), respectively).

According to the FDA (https://www.fda.gov/downloads/Drugs/GuidanceComplianceRegulatoryInformation/Guidan ces/UCM579751.pdf), the contents of which is incorporated herein by reference), NDAs and ANDAs can be divided into the following four categories:

(1) A “stand-alone NDA” is an application submitted under section 505(b)(1) and approved under section 505(c) of the FD&C Act that contains full reports of investigations of safety and effectiveness that were conducted by or for the applicant or for which the applicant has a right of reference or use.

(2) A 505(b)(2) application is an NDA submitted under section 505(b)(1) and approved under section 505(c) of the FD&C Act that contains full reports of investigations of safety and effectiveness, where at least some of the information required for approval comes from studies not conducted by or for the applicant and for which the applicant has not obtained a right of reference or use.

(3) An ANDA is an application for a duplicate of a previously approved drug product that was submitted and approved under section 505(j) of the FD&C Act. An ANDA relies on FDA's finding that the previously approved drug product, i.e., the reference listed drug (RLD), is safe and effective. An ANDA generally must contain information to show that the proposed generic product (a) is the same as the RLD with respect to the active ingredient(s), conditions of use, route of administration, dosage form, strength, and labeling (with certain permissible differences) and (b) is bioequivalent to the RLD. An ANDA may not be submitted if studies are necessary to establish the safety and effectiveness of the proposed product.

(4) A petitioned ANDA is a type of ANDA for a drug product that differs from the RLD in its dosage form, route of administration, strength, or active ingredient (in a product with more than one active ingredient) and for which FDA has determined, in response to a petition submitted under section 505(j)(2)(C) of the FD&C Act (suitability petition), that studies are not necessary to establish the safety and effectiveness of the proposed drug product.

A scientific premise underlying the Hatch-Waxman Amendments is that a drug product approved in an ANDA under section 505(j) of the FD&C Act is presumed to be therapeutically equivalent to its RLD. Products classified as therapeutically equivalent can be substituted with the full expectation that the substituted product will produce the same clinical effect and safety profile as the prescribed product when administered to patients under the conditions specified in the labeling. In contrast to an ANDA, a 505(b)(2) application allows greater flexibility as to the characteristics of the proposed product. A 505(b)(2) application will not necessarily be rated therapeutically equivalent to the listed drug it references upon approval.

The term “therapeutically equivalent to a reference listed drug” is means that the drug product is a generic equivalent, i.e., pharmaceutical equivalents, of the reference listed drug product and, as such, is rated an AB therapeutic equivalent to the reference listed drug product by the FDA whereby actual or potential bioequivalence problems have been resolved with adequate in vivo and/or in vitro evidence supporting bioequivalence.

“Pharmaceutical equivalents” means drug products in identical dosage forms and route(s) of administration that contain identical amounts of the identical active drug ingredient as the reference listed drug.

FDA classifies as therapeutically equivalent those products that meet the following general criteria: (1) they are approved as safe and effective; (2) they are pharmaceutical equivalents in that they (a) contain identical amounts of the same active drug ingredient in the same dosage form and route of administration, and (b) meet compendial or other applicable standards of strength, quality, purity, and identity; (3) they are bioequivalent in that (a) they do not present a known or potential bioequivalence problem, and they meet an acceptable in vitro standard, or (b) if they do present such a known or potential problem, they are shown to meet an appropriate bioequivalence standard; (4) they are adequately labeled; and (5) they are manufactured in compliance with Current Good Manufacturing Practice regulations

The term “bioequivalent” or “bioequivalence” is the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Section 505 (j)(8)(B) of the FD&C Act describes one set of conditions under which a test and reference listed drug shall be considered bioequivalent:

the rate and extent of absorption of the [test] drug do not show a significant difference from the rate and extent of absorption of the [reference] drug when administered at the same molar dose of the therapeutic ingredient under similar experimental conditions in either a single dose or multiple doses; or

the extent of absorption of the [test] drug does not show a significant difference from the extent of absorption of the [reference] drug when administered at the same molar dose of the therapeutic ingredient under similar experimental conditions in either a single dose or multiple doses and the difference from the [reference] drug in the rate of absorption of the drug is intentional, is reflected in its proposed labeling, is not essential to the attainment of effective body drug concentrations on chronic use, and is considered medically insignificant for the drug.

Where these above methods are not applicable (e.g., for drug products that are not intended to be absorbed into the bloodstream), other scientifically valid in vivo or in vitro test methods to demonstrate bioequivalence may be appropriate.

For example, bioequivalence may sometimes be demonstrated using an in vitro bioequivalence standard, especially when such an in vitro test has been correlated with human in vivo bioavailability data. In other situations, bioequivalence may sometimes be demonstrated through comparative clinical trials or pharmacodynamic studies.

The terms “sale” or “selling” means transferring a drug product, e.g., a pharmaceutical composition or an oral dosage form, from a seller to a buyer.

The term “offering for sale” means the proposal of a sale by a seller to a buyer for a drug product, e.g., a pharmaceutical composition and an oral dosage form.

### Routes of Administration and Pharmaceutical Compositions

Therapeutic agents described herein are administered in any suitable manner or suitable formulation. Suitable routes of administration of the therapeutic agents include, but are not limited to, oral and parenteral (e.g., intravenous, subcutaneous, intramuscular). All formulations are in dosages suitable for administration to a human. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

The term “safe and effective amount” refers to an amount of an active ingredient that elicits the desired biological or medicinal response in a subject's biological system without the risks outweighing the benefits of such response in accordance with the Federal Food, Drug, and Cosmetic Act, as amended (secs. 201-902, 52 Stat. 1040 et seq., as amended; 21 U.S.C. §§ 321-392). Safety is often measured by toxicity testing to determine the highest tolerable dose or the optimal dose of an active pharmaceutical ingredient needed to achieve the desired benefit.

Studies that look at safety also seek to identify any potential adverse effects that may result from exposure to the drug. Efficacy is often measured by determining whether an active pharmaceutical ingredient demonstrates a health benefit over a placebo or other intervention when tested in an appropriate situation, such as a tightly controlled clinical trial.

The term “acceptable” with respect to a formulation, composition or ingredient, as used herein, means that the beneficial effects of that formulation, composition or ingredient on the general health of the male human being treated substantially outweigh its detrimental effects, to the extent any exist.

In an embodiment of the present invention, the label for the RLD contains instructions for administering the drug product to a person unable to swallow whole tablets. The drug product contains an active pharmaceutical ingredient selected from apalutamide, enzalutamide or darolutamide. In an embodiment of the present invention, the active pharmaceutical ingredient is apalutamide.

In some embodiments, administration of a safe and effective amount of the anti-androgen results in no more than a grade 2 adverse event. In other embodiments, administration of a safe and effective amount of anti-androgen results in no more than a grade 3 adverse event. In other embodiments, administration of a safe and effective amount of anti-androgen results in no more than a grade 4 adverse event.

In some embodiments, the anti-androgen is present in a solid oral dosage form. In some embodiments, the anti-androgen is formulated as a tablet. In some embodiments, the anti-androgen is apalutamide. In some embodiments, the anti-androgen is enzalutamide. Solid oral dosage forms containing either apalutamide or enzalutamide may be provided as soft gel capsules as disclosed in WO2014113260 and CN104857157, each of which is incorporated herein by reference, or as tablets as disclosed in WO2016090098, WO2016090101, WO2016090105, and WO2014043208, each of which is incorporated herein by reference. Techniques suitable for preparing solid oral dosage forms of the present invention are described in Remington's Pharmaceutical Sciences, 18th edition, edited by AR. Gennaro, 1990, Chapter 89, and in Remington—The Science, and Practice of Pharmacy, 21st edition, 2005, Chapter 45.

To prepare the pharmaceutical compositions of this invention, the active pharmaceutical ingredient is intimately admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

In solid oral preparations such as, for example, dry powders for reconstitution or inhalation, granules, capsules, caplets, gelcaps, pills and tablets (each including immediate release, timed release and sustained release formulations), suitable carriers and additives include but are not limited to diluents, granulating agents, lubricants, binders, glidants, disintegrating agents and the like. Because of their ease of administration, tablets and capsules represent the most advantageous oral dosage unit form, in which case solid pharmaceutical carriers are obviously employed. If desired, tablets may be sugar coated, gelatin coated, film coated or enteric coated by standard techniques.

Preferably these compositions are in unit dosage forms from such as tablets, pills, capsules, dry powders for reconstitution or inhalation, granules, lozenges, sterile solutions or suspensions, metered aerosol or liquid sprays, drops, or suppositories for administration by oral, intranasal, sublingual, intraocular, transdermal, rectal, vaginal, dry powder inhaler or other inhalation or insufflation means.

These formulations are manufactured by conventional formulation techniques. For preparing solid pharmaceutical compositions such as tablets, the principal active ingredient is mixed with a pharmaceutical carrier, e.g., conventional tableting ingredients such as diluents, binders, adhesives, disintegrants, lubricants, antiadherents, and gildants. Suitable diluents include, but are not limited to, starch (i.e. corn, wheat, or potato starch, which may be hydrolized), lactose (granulated, spray dried or anhydrous), sucrose, sucrose-based diluents (confectioner's sugar; sucrose plus about 7 to 10 weight percent invert sugar; sucrose plus about 3 weight percent modified dextrins; sucrose plus invert sugar, about 4 weight percent invert sugar, about 0.1 to 0.2 weight percent cornstarch and magnesium stearate), dextrose, inositol, mannitol, sorbitol, microcrystalline cellulose (i.e. AVICEL microcrystalline cellulose available from FMC Corp.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (i.e. alginic acid and salts thereof, magnesium aluminum silicate, hydroxyethylcellulose [i.e. TYLOSE available from Hoechst Celanese], polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like. Suitable lubricants and antiadherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like. Suitable gildants include, but are not limited to, talc, cornstarch, silica (i.e. CAB-O-SIL silica available from Cabot, SYLOID silica available from W. R. Grace/Davison, and AEROSIL silica available from Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

Binders suitable for use in the pharmaceutical compositions provided herein include, but are not limited to, starches, cellulose, and its derivatives (e.g., ethylcellulose, cellulose acetate, carboxymethyl cellulose calcium, sodium carboxymethyl cellulose, methylcellulose, hydroxypropyl methylcellulose), polyvinyl, prolidone, and mixtures thereof.

Examples of fillers suitable for use in the pharmaceutical compositions provided herein include, but are not limited to, microcrystalline cellulose, powdered cellulose, mannitol, lactose, calcium phosphate, starch, pre gelatinized starch, and mixtures thereof.

The binder or filler in pharmaceutical compositions is typically present in from about 50 to about 99 weight percent of the pharmaceutical composition or dosage form.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant. Disintegrants that can be used in the pharmaceutical compositions provided herein include, but are not limited to, croscarmellose sodium, crospovidone, sodium starch glycolate, potato or tapioca starch, pregelatinized starch, other starches, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, polyethylene glycol, other glycols, stearic acid, sodium lauryl sulfate, sodium stearyl fumarate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Lubricants are typically used in an amount of less than about 1 weight percent of the pharmaceutical compositions or dosage forms into which they are incorporated.

Compressed tablet formulations may optionally be film-coated to provide color, light protection, and/or taste-masking. Tablets may also be coated so as to modulate the onset, and/or rate of release in the gastrointestinal tract, so as to optimize or maximize the biological exposure of the patient to the API.

Hard capsule formulations may be produced by filling a blend or granulation of apalutamide or enzalutamide into shells consisting of, for example, gelatin, or hypromellose. Soft gel capsule formulations may be produced. Pharmaceutical compositions intended for oral use may be prepared from the solid dispersion formulations, and blended materials described above in accordance with the methods described herein, and other methods known to the art for the manufacture of pharmaceutical compositions. Such compositions may further contain one or more agents selected from the group consisting of sweetening agents, flavoring agents, coloring agents, and preserving agents in order to provide pharmaceutically elegant and palatable preparations.

Tablets may contain the active ingredient in admixture with non-toxic pharmaceutically acceptable excipients that are suitable for the manufacture of tablets. These excipients may be for example, inert diluents, granulating, and disintegrating agents, binding agents, glidants, lubricating agents, and antioxidants, for example, propyl gallate, butylated hydroxyanisole, and butylated hydroxy toluene. The tablets may be uncoated or they may be film coated to modify their appearance or may be coated with a functional coat to delay disintegration, and absorption in the gastrointestinal tract, and thereby provide a sustained action over a longer period.

The drug product of present invention may be produced by any number of methods known to those skilled in the art. Examples for apalutamide drug product include those described in International application numbers PCT/US2015/063661, PCT/US2015/063667, PCT/US2015/063671 and for enzalutamide drug product, international application number PCT/2013/059223, each of the foregoing patent applications are fully incorporated herein by reference. In addition, the drug product may contain apalutamide, enzalutamide, darolutamide as single agent formulation or in combination with other active pharmaceutical compounds. The drug product may be a tablet, capsule, powder or other form suitable for dispersing or dissolving in a suitable vehicle, for example applesauce, green tea extract, orange juice or yogurt, preferably applesauce.

The term “suitable vehicle” means any fluid or semi-liquid edible substance, where the water content of the edible substance is greater than or equal to about 70%; and wherein the pH of the edible substance is in the range of from about pH 3 to about pH 10; preferably in the range of from about pH 3 to about pH 8, more preferably in the range of from about pH 4.5 to about pH 7.5. In certain embodiments of the present invention, the suitable vehicle does not contain alcohol (i.e. is not an alcoholic beverage). In certain embodiments of the present invention, the suitable vehicle is not carbonated (e.g. is not a soda, tonic water, and the like). In certain embodiments of the present invention, the suitable vehicle is a liquid. In certain embodiments of the present invention, the suitable vehicle is a liquid or semi-solid. In certain embodiments of the present invention, the suitable vehicle is a semi-solid.

In certain embodiments of the present invention, the suitable vehicle is a liquid. Examples include, but are not limited to water, tea or tea extract (including but not limited to black tea, green tea, herbal tea such as chamomile, mint, rooibos, and the like), coffee, milk, coconut milk, fruit juice (such as orange, apple, pear, grape, tomato, and the like), lemonade, plant based milks (such as oat, soy, almond, and the like), sports drinks, electrolyte drinks, and the like.

In certain embodiments of the present invention, the suitable vehicle is a semi-solid. Examples include, but are not limited to applesauce, fruit puree (such as apple, banana, carrot, and the like), yogurt (such as plain, flavored, with fruit, and the like), jam (such as strawberry, grape, orange, and the like) and the like.

As used herein, unless otherwise noted, the terms “tea” and “tea extract” mean a solution prepared by infusing the dried and optionally crushed leaves of a tea plant (for example, Camellia sinensis) in boiling water. The terms “tea” or “tea extract” shall further include “herbal tea” or “herbal tea extract”, which mean a solution prepared by infusing the dried and optionally crushed leaves of an herb plant in boiling water, for example the chamomile, rooibos, or mint plant.

Wherein certain embodiments of the present invention, the pharmaceutical agent, drug, or active ingredient (e.g. apalutamide, enzalutamide, darolutamide), as a tablet, is dispersed in a suitable vehicle (for example applesauce, yogurt, fruit juice, tea, and the like) the term “dispersed” as used herein refers to the physical state resulting from the action of blending whole tablets with the suitable vehicle (for example, yogurt, fruit juice, tea, and the like) according to known methods, for example, according to the protocols or procedures described hereinafter in Examples 1-8 to produce a homogenous drug-vehicle mixture. One skilled in the art will further recognize that wherein apalutamide is dispersed into a suitable vehicle, the resulting dispersion is a mixture of apalutamide and said suitable vehicle.

One skilled in the art will recognize that wherein the methods of treatment, pharmaceutical compositions and methods for the preparation of pharmaceutical compositions of the present invention, “apalutamide” is mixed (e.g. dispersed) in a suitable vehicle (such as applesauce, tea, yogurt, orange juice, or the like) said “apalutamide” is present as an oral dosage form (for example, as whole tablets, as whole tablets as described in Example 1 which follows hereinafter, and the like).

Compositions for oral use may also be presented as capsules (e.g., hard gelatin) wherein the active ingredient is mixed with an inert solid diluent, for example, calcium carbonate, calcium phosphate or starch, or as soft gelatin capsules wherein the active ingredient is mixed with liquids or semisolids, for example, peanut oil, liquid paraffin, fractionated glycerides, surfactants or olive oil. Aqueous suspensions contain the active materials in mixture with excipients suitable for the manufacture of aqueous suspensions. Dispersible powders and granules suitable for preparation of an aqueous suspension by the addition of water provide the active ingredient in mixture with a dispersing or wetting agent, suspending agent, and one or more preservatives. In certain embodiments of the invention, the pharmaceutical compositions of the invention include a diluent system, disintegrant, salt, lubricant, glidant, and filmcoat, at concentrations of from about 3% w/w to about 58% w/w, from about 4% w/w to about 20% w/w, from about 4% w/w to about 20% w/w, from about 0.5% w/w to about 4% w/w, from about 0% w/w to about 2% w/w, and from about 1% w/w to about 5% w/w respectively, or at from about 18% w/w to about 40% w/w, from about 7% w/w to about 15% w/w, from about 7% w/w to about 18% w/w, from about 1.0% w/w to about 3.0%, from about 0.1% w/w to about 1.0% w/w, and from about 2.0% w/w to about 4.0% w/w, respectively. In certain embodiments, the solid dispersion formulations are blended with a diluent, one or more disintegrating agents, lubricants, and glidants. An exemplary blended composition or oral dosage form includes mannitol, microcrystalline cellulose, croscarmellose sodium, sodium chloride, colloidal silica, sodium stearyl fumarate, and magnesium stearate.

The disintegrant may be present in a concentration from about 4% w/w to about 20% w/w or from about 7% w/w to about 15% w/w. A salt may be also present, which may be sodium chloride, potassium chloride or a combination thereof. The combination of salts and disintegrant is present at a concentration from about 5% w/w to about 35% w/w of the final pharmaceutical composition.

In certain embodiments, inactive ingredients of the core tablet are: colloidal anhydrous silica, croscarmellose sodium, hydroxypropyl methylcellulose-acetate succinate, magnesium stearate, microcrystalline cellulose, and silicified microcrystalline cellulose. In other embodiments, the tablets are finished with a film-coating consisting of the following excipients: iron oxide black, iron oxide yellow, polyethylene glycol, polyvinyl alcohol, talc, and titanium dioxide.

In other embodiments, a single unit dosage of the pharmaceutical composition comprises, consists of, or consists essentially of about 60 mg of apalutamide. In some embodiments, multiple doses of the single unit dosage pharmaceutical composition comprising, consisting of, or consisting essentially of about 60 mg of apalutamide, e.g., 4 multiple or individual unit dosage forms, are administered to the human. The total daily dose of apalutamide may be about 240 mg per day.

In some embodiments, a single unit dosage of the pharmaceutical composition comprises, consists of, or consists essentially of about 40 mg of enzalutamide. In some embodiments, multiple doses of the single unit dosage pharmaceutical composition comprising, consisting of, or consisting essentially of about 40 mg of enzalutamide, e.g., 4 multiple or individual unit dosage forms, are administered to the human. The total daily dose of enzalutamide may be about 160 mg per day.

In still further embodiments, a single unit dosage of the pharmaceutical composition comprises, consists of, or consists essentially of about 300 mg of darolutamide. In some embodiments, multiple doses of the single unit dosage pharmaceutical composition comprising, consisting of, or consisting essentially of about 300 mg of enzalutamide, e.g., 2 multiple or individual unit dosage forms, are administered to the human. The total daily dose of darolutamide may be about 1200 mg per day.

All formulations for oral administration are in dosage form suitable for such administration.

### Methods of Dosing and Treatment Regimens

In one aspect, described herein are methods of treating non-metastatic castration-resistant prostate cancer comprising, consisting of, or consisting essentially of administering a safe and effective amount of an anti-androgen to a male human with a non-metastatic castration-resistant prostate cancer, wherein the apalutamide or enzalutamide is administered orally. In some embodiments, the anti-androgen is administered daily. In some embodiments, the anti-androgen is administered twice-a-day. In some embodiments, the anti-androgen is administered three times a day. In some embodiments, the anti-androgen is administered four times a day. In some embodiments, the apalutamide is administered every other day. In some embodiments, the antiandrogen is administered weekly. In some embodiments, the anti-androgen is administered twice a week. In some embodiments, the anti-androgen is administered every other week. In some embodiments, the anti-androgen is administered orally on a continuous daily dosage schedule.

In one embodiment, the desired dose is conveniently presented in a single dose or in divided doses administered simultaneously (or over a short period of time) or at appropriate intervals, for example as two, three, four or more sub-doses per day to patient in need thereof. In some embodiments, the anti-androgen is conveniently presented in divided doses that are administered simultaneously (or over a short period of time) once a day. In some embodiments, the anti-androgen is conveniently presented in divided doses that are administered in equal portions twice-a-day. In some embodiments, the anti-androgen is conveniently presented in divided doses that are administered in equal portions three times a day. In some embodiments, the anti-androgen is conveniently presented in divided doses that are administered in equal portions four times a day.

In some embodiments, the anti-androgen is a second-generation anti-androgen. In certain embodiments, the anti-androgen is enzalutamide or apalutamide. In some embodiments, the antiandrogen is enzalutamide. In some embodiments, the anti-androgen is apalutamide. In some embodiments, the anti-androgen is darolutamide.

In general, doses of apalutamide employed for treatment of the diseases or conditions described herein in humans are typically in the range of 10 mg to 1000 mg per day. In some embodiments, apalutamide, enzalutamide or darolutamide is administered orally to the human at a dose of about 30 mg per day to about 1200 mg per day. In some embodiments, apalutamide is administered orally to the human at a dose of about 30 mg per day to about 600 mg per day. In some embodiments, apalutamide is administered orally to the human at a dose of about 30 mg per day, about 60 mg per day, about 90 mg per day, about 120 mg per day, about 160 mg per day, about 180 mg per day, about 240 mg per day, about 300 mg per day, about 390 mg per day, about 480 mg per day, about 600 mg per day, about 780 mg per day, about 960 mg per day, or about 1200 mg per day.

In some embodiments, apalutamide is administered orally to the human at a dose of about 240 mg per day. In some embodiments, greater than 240 mg per day of apalutamide is administered to the human. In some embodiments, the apalutamide is administered orally to the human at a dose of about 60 mg four times per day. In some embodiments, apalutamide is administered orally to the human on a continuous daily dosing schedule.

In some embodiments, the enzalutamide is administered orally at a dose of about 160 mg per day. In some embodiments, greater than 160 mg per day of enzalutamide is administered.

In some embodiments, the darolutamide is administered orally at a dose of about 1200 mg per day. In some embodiments, greater than 1200 mg per day of darolutamide is administered.

In certain embodiments wherein improvement in the status of the disease or condition in the human is not observed, the daily dose of anti-androgen is increased. In some embodiments, a once-a-day dosing schedule is changed to a twice-a-day dosing schedule. In some embodiments, a three times a day dosing schedule is employed to increase the amount of anti-androgen that is administered.

In some embodiments, the amount of anti-androgen that is given to the human varies depending upon factors such as, but not limited to, condition and severity of the disease or condition, and the identity (e.g., weight) of the human, and the particular additional therapeutic agents that are administered (if applicable).

In certain embodiments, the dose of anti-androgen, e.g., apalutamide, enzalutamide, or darolutamide is reduced when co-administered with one or more of:

(a) a CYP2C8 inhibitor, preferably gemfibrozil or clopidogrel; or

(b) a CYP3A4 inhibitor, preferably ketoconazole or ritonavir.

In some embodiments, the apalutamide is not co-administered with:

(a) medications that are primarily metabolized by CYP3A4, preferably darunavir, felodipine, midazolam or simvastatin;

(b) medications that are primarily metabolized by CYP2C19, preferably diazepam or omeprazole;

(c) medications that are primarily metabolized by CYP2C9, preferably warfarin or phenytoin; or

(d) medications that are substrates of UGT, preferably levothyroxine or valproic acid.

In further embodiments, the apalutamide is not co-administered with:

(a) medications that are P-gp substrates, preferably fexofenadine, colchicine, dabigatran etexilate or digoxin; or

(b) BCRP/OATP1B1 substrates, preferably lapatinib, methotrexate, rosuvastatin, or repaglinide.

In further embodiments, a male human having said non-metastatic castration-resistant prostate cancer has received at least one prior therapy for the treatment of cancer, optionally wherein the prior therapy for the treatment of cancer is bicalutamine or flutamide. In still further embodiments, a male human having said non-metastatic castration-resistant prostate cancer is treatment naïve.

The following example(s) are provided for illustrative purposes only and not to limit the scope of the claims provided herein.

### Example 1

### Phase I Clinical Trial Protocol: A Randomized, Open-Label, Two-Way, Crossover Study to Evaluate the Relative Bioavailability of Apalutamide Administered Orally as Whole Tablets and as a Mixture in Applesauce in Healthy Subjects

Abbreviations used throughout this Example are as follows:
AE adverse event
ALT alanine aminotransferase
AR androgen receptor
CI confidence interval
CNS central nervous system
CRC child-resistant closures
CRF case report form
CRPC castration-resistant prostate cancer
CV coefficient of variation
CYP cytochrome P450 isozyme
CSR clinical study report
ECG electrocardiogram
eDC electronic data capture
FDA Food and Drug Administration
GCP Good Clinical Practice
HBsAg hepatitis B surface antigen
HCV hepatitis C virus
HDPE high-density polyethylene
HIV human immunodeficiency virus
ICF Informed consent form
ICH International Council for Harmonisation of Technical Requirements for Pharmaceuticals for Human Use
IEC/IRB Independent Ethics Committee/Institutional Review Board
LC-MS/MS Liquid chromatography/mass spectrometry/mass spectrometry
LH Luteinizing hormone
LLOQ Lower limit of quantification
MoCRPC non-metastatic castration-resistant prostate cancer
NCI-CTCAE National Cancer Institute—Common Terminology Criteria for Adverse Events
NOAEL no observed adverse effect level
PK pharmacokinetic(s)
PQC Product quality complaint
PSA prostate-specific antigen
QTcB QT corrected according to Bazett's formula
QTcF QT corrected according to Fridericia's formula
SD standard deviation
TEAE treatment-emergent adverse event
TSH thyroid-stimulating hormone

### Synopsis

Study Drug: Apalutamide is an orally available, non-steroidal small molecule, which acts as a potent and selective antagonist of the androgen receptor (AR), currently being developed for the treatment of prostate cancer.

This study was designed to determine the bioavailability of apalutamide administered orally as a mixture in applesauce relative to whole tablets, both under fasted conditions. As a drug administration vehicle, applesauce can provide a convenient medium for the dispersion of apalutamide tablets that avoids the need to crush tablets via mortar and pestle, tools that are not commonly available in a home care setting.

### Objectives and Hypothesis

Primary Objective: The primary objective was to determine the bioavailability of apalutamide tablets administered orally as dispersed tablets in applesauce relative to whole tablets under fasting conditions in healthy male subjects.

Secondary Objective: The secondary objective was to assess the safety profile of apalutamide following single dose administration as whole tablets and as dispersed tablets in applesauce.

Exploratory Objective: The exploratory objective was to evaluate the palatability of apalutamide-applesauce mixture.

### Overview of Study Design

This was a randomized, open-label, balanced, single dose, two-treatment, two-period, two-sequence, crossover relative bioavailability study. Healthy male subjects were administered a single dose of apalutamide 240 mg on 2 separate occasions either as whole tablets or as dispersed tablets in applesauce.

There was a screening phase (within 21 days before study drug administration in the first period); an open-label treatment phase consisting of 2 single-dose treatment periods; and end of study or early withdrawal assessments done upon completion of the 168-hour pharmacokinetic (PK) sampling on Day 8 of period 2 or upon early withdrawal. Successive study drug administrations (Treatment Periods 1 and 2) were separated by a washout interval of at least 42 days and no more than 56 days between doses.

Healthy male subjects were randomly assigned to 1 of 2 treatment sequences by following the randomization scheme as given below. A single dose of 240 mg apalutamide was administered as either whole tablets (Treatment A, reference) or dispersed mixture in applesauce (Treatment B, test) to each subject on Day 1 of each treatment period under fasted conditions.

TABLE E1
Treatment Sequence for Randomization Scheme

| Sequence | No. of | Period | Period |
|---|---|---|---|
| Number | Subjects | 1 | 2 |
| 1 | 6 | A | B |
| 2 | 6 | B | A |

In each treatment period, subjects entered the study center in the morning on Day −1 and remained there until after the collection of the 72-hour PK sample on Day 4 if the investigator considers the subject ready for discharge. On Day 1, a single dose of the appropriate study drug preparation was administered to each subject in the morning followed by sequential collection of blood samples over 168 hours (Day 8) for the determination of apalutamide plasma concentrations. Subjects returned daily to the study center for PK sampling from Day 5 to Day 8.

For Treatment B, a taste questionnaire was completed by the subject after study drug intake. The questionnaire consisted of a visual analogue scale to rate 3 items (sweetness, bitterness, and smell) as well as overall acceptability (not acceptable or acceptable).

Subject's safety was monitored throughout the study. Details on the timing of the treatment and assessments were as provided in the Time and Events Schedule (See FIG. 1).

Duration of Study: The duration of participation in the study for an individual subject was approximately 84 days (including screening).

### Subject Selection

Number of subjects: Approximately 12 healthy male subjects were randomly assigned in this study to one of the two treatment sequences based on a computer-generated randomization schedule prepared before the study by or under the supervision of the sponsor.

Main Criteria for Inclusion: Healthy men between 18 and 55 years of age, inclusive; body mass index (BMI) between 18 and 30 kg/m², inclusive, and a body weight of not less than 50 kg.

### Study Drugs, Formulation, Dose, and Mode of Administration

Study Drug: A dose of 240 mg (4×60 mg tablets) apalutamide was given in each treatment period under fasted conditions.

Treatment A (Reference treatment): 240 mg of apalutamide commercial formulation given as a single dose of 4×60 mg tablets swallowed whole under fasted conditions.

Treatment B (Test treatment): 240 mg of apalutamide commercial formulation given as a single dose of 4×60 mg tablets as a dispersed mixture in applesauce under fasted conditions.

Before each study drug administration, subjects fasted from food and fluids (excluding noncarbonated water) for at least 10 hours. Noncarbonated water was allowed up to 1 hour before study drug administration.

All study drug were taken in the morning on Day 1 of each treatment period. Study drug was taken with 240 mL of noncarbonated water (Treatment A) or mixed with an approximate volume of 4 oz or 120 mL of applesauce supplemented by a container rinse of 120 mL of noncarbonated water (Treatment B). For Treatment B, the study drug-applesauce mixture was prepared by a pharmaceutical technician or pharmacist. An additional 50 mL of water for either treatments was allowed if necessary. Study drug of Treatment B was be ingested within 5 minutes. Treatment A was be swallowed whole and not chewed, divided, dissolved, or crushed.

Subjects continued fasting until 4 hours after study drug administration. At approximately 1 hour after dosing (but not earlier) all subjects were to drink 1 glass (approximately 240 mL) of water; drinking of water was allowed from then onwards. A standardized lunch was served on Day 1 of each treatment period for all subjects after collection of the 4-hour PK blood sample. Standard meals and snacks were provided while the subjects are at the study center.

### Study Evaluations

FIG. 1 lists the Time and Event Schedule for the Clinical Trial.

Pharmacokinetics: Serial venous blood samples were collected at pre-dose and over 168 hours (Day 8) after study drug administration for determination of plasma concentrations of apalutamide. The following PK parameters for apalutamide were calculated: Cₘₐₓ, tₘₐₓ, AUC_{0-72 h}, and AUC_{0-168 h}. Additional PK parameters could be included if deemed appropriate.

Safety: Safety was evaluated throughout the study by means of adverse events (AEs), laboratory safety (hematology and serum chemistry), electrocardiograms (ECGs), vital signs (blood pressure, pulse, respiratory rate, and body temperature), and physical examinations (see the Time and Events Schedule in FIG. 1 for details).

Taste: Feedback regarding palatability of the apalutamide dispersed in applesauce (Treatment B) was collected via a taste questionnaire conducted within 30 min after intake of Treatment B. The questionnaire consisted of a visual analogue scale to rate 3 items (sweetness, bitterness, and smell) as well as overall acceptability (not acceptable or acceptable).

### Statistical Methods

Sample Size Determination: More recent studies with similar study indicated the intra-subject coefficient of variation (CV) for Cₘₐₓ and AUCs of apalutamide ranged 11%-12% and 4%-7%, respectively.

Applying a conservative assumption for intra-subject CV of 12% for Cₘₐₓ and 7% for AUCs, respectively, a sample size of 10 subjects in the current study was determined to be sufficient for the point estimates of the geometric mean ratios of Cₘₐₓ and AUCs to fall within (91%, 110%) and (95%, 105%) of the true value respectively, with 90% confidence.

Assuming a dropout rate of 17%, approximately 12 subjects were randomized to ensure at least 10 PK evaluable subjects complete the study where a PK evaluable subject is defined by having sufficient and interpretable PK assessments to calculate at least 1 noncompartmental PK parameter. If the number of PK evaluable subjects who complete the study were to fall to less than 10, additional subjects could be enrolled for replacement by assigning them to the same treatment sequence as the subjects being replaced. Replacement subjects would start with Period 1. Subjects who prematurely discontinued from the study due to drug related AEs or AEs which occur in relation to study procedures were not replaced.

Pharmacokinetics: The PK population included all subjects who have sufficient and interpretable concentration-time data.

Data was listed for all subjects with available study drug plasma concentrations per treatment. All concentrations below lower limit of quantification (LLOQ) or missing data was be labeled as such in the concentration data listings. Concentrations below LLOQ were be treated as zero in the summary statistics and for the calculation of PK parameters. All subjects and samples excluded from the analysis were clearly documented in the clinical study report (CSR).

Factors that may influence the study drug plasma concentrations (eg, vomiting, concomitant medication, fever, high predose concentration) were checked. If an influencing factor was present, a decision was made by the responsible pharmacokineticist, as to whether to include or exclude the specific sample or subject.

For each treatment group, descriptive statistics, including arithmetic mean, standard deviation (SD), coefficient of variation, geometric mean, median, minimum, and maximum were calculated for all individual derived PK parameters including exposure information of apalutamide.

Graphical representations of the results included, but was not limited to, the following graphs: Log-linear and linear-linear plasma concentration-time profiles for each individual. Log-linear and linear-linear plasma concentration-time profiles for the mean values per treatment.

The primary objective of the statistical analysis was to determine the relative bioavailability of Treatment B with respect to the reference Treatment A. The primary parameters of interest for the statistical analysis were Cₘₐₓ, AUC_{0-72 h}, and AUC_{0-168 h}. If one of the PK parameters could not be determined for a given subject in 1 or more periods, the subject's data was included in the statistical analysis of that particular PK parameter. The analysis was performed on log-transformed PK parameters.

A mixed effect model that includes treatment, period, and treatment sequence as fixed effects, and subject as a random effect, was used to estimate the least squares means and intrasubject variance. Using these estimated least squares means and intrasubject variance, the point estimate and 90% CIs for the difference in means on a log scale between test and reference were constructed. The limits of the CIs were retransformed using antilogarithms to obtain 90% CIs for the geometric mean ratios of Cₘₐₓ, and AUC_{0-168 h} of the test to reference formulation. A similar analysis was conducted for AUC_{0-72 h} for supplemental purpose.

Additional statistical analysis may be performed if deemed appropriate.

Safety: Baseline for all laboratory evaluations, 12-lead ECG measurements, and vital signs were defined as the last evaluation done before the first study drug administration. Safety was evaluated by examining the incidence and type of adverse events, and changes in clinical laboratory test values, physical examination results, 12-lead ECGs, and vital sign measurements from the screening phase through study completion, including the washout intervals and the End-of-Study visit.

Taste: Questionaire data (sweetness, bitterness, smell, and overall acceptability) was summarized using descriptive statistics. (based on the taste questionnaire as shown in FIG. 2.)

### Detailed Clinical Trial Plan

### Introduction

Apalutamide (ERLEADA) is an androgen receptor (AR) inhibitor that is approved for the treatment of patients with non-metastatic castration-resistant prostate cancer (MoCRPC) in the United States and Canada. The approved dose is 240 mg (4×60 mg tablets) administered once daily with or without food. The tablets should be swallowed whole as instructed in the prescriber information.

The trending voice of customer analysis suggests that there is a need to provide dosing information for patients who have swallowing difficulties due to the size of the ERLEADA tablet (17×9 mm). The company is evaluating whether an alternative mode of administration using soft food as vehicle for the commercial tablets of apalutamide will negatively affect bioavailability of the formulation. As apalutamide tablets rapidly disperse in aqueous media, an alternative mode of administration involving dispersed tablets mixed in applesauce is being considered. With this, it is anticipated that having an alternative mode of administration for apalutamide as dispersed tablets in a soft food vehicle such as applesauce will positively impact patient adherence and acceptance.

### Background: Absorption, Distribution, Metabolism, and Excretion (ADME) Characteristics

Apalutamide is a Biopharmaceutics Classification System Class II drug (highly permeable and poorly soluble). The commercialized formulation is a spray dried powder of amorphous apalutamide. The absolute bioavailability (Fabs) of a single dose of 240 mg apalutamide was determined to be approximately 100% (Fabs=1.11).

Following administration of a single 240-mg dose as a tablet formulation to healthy subjects, apalutamide reached Cₘₐₓ with a median Tₘₐₓ of about 2 to 3 hours. Mean single dose plasma Cₘₐₓ values were approximately 2.4 μg/mL (N=15), while mean values for AUC₂₈ were 225 μg*h/mL (N=15) at the 240 mg dose. Mean elimination half-life (t_{1/2}) of apalutamide was about 162 hours (˜7 days).

Administration of apalutamide tablets to healthy subjects with a high-fat meal resulted in a slight decrease in the rate of absorption based on Cₘₐₓ and Tₘₐₓ. Median Tₘₐₓ of apalutamide was delayed by 2 hours and Cₘₐₓ was approximately 16% (90% confidence interval [CI]=74.9% to 94.1%) lower compared to fasted conditions. The extent of absorption based on AUC_{∞} remained unchanged under fed conditions, with 90% CI for the fed/fasted ratio within 80% to 125%.

Results from the single-dose ¹⁴C-apalutamide absolute bioavailability and microtracer study indicated that 88.9% of the total administered ¹⁴C-radioactive dose was recovered, of which the majority was recovered up to 42 days. The major route of excretion of total ¹⁴C-radioactivity was urine (arithmetic mean=64.2%), with less than 4% of the administered dose being excreted as unchanged apalutamide and 4-[7-[6-cyano-5-(trifluoromethyl)-3-pyridinyl]-8-oxo-6-thioxo-5,7-diazaspiro[3.4]oct-5-yl]-2-fluoro-benzamide. The remainder of ¹⁴C-radioactivity was excreted via feces (arithmetic mean=24.3%).

Efficacy and Safety in Subjects with Prostate Cancer:

The efficacy of apalutamide was demonstrated in patients with prostate cancer in the Phase 3 SPARTAN registration study. This study was a randomized, double-blind, placebo-controlled, multicenter trial that evaluated apalutamide treatment in patients with high-risk nmCRPC. A total of 1207 patients were randomized by a 2:1 ratio to receive 240 mg apalutamide once daily (806 patients) or placebo (401 patients). All patients continued to receive ongoing androgen-deprivation therapy (GnRHa or surgical castration). This study was the first to demonstrate a significant longer median metastasis-free survival (2 years over placebo) in apalutamide-treated patients compared with placebo-treated patients, with consistent benefit for apalutamide across all secondary endpoints, including time to symptomatic progression.

A confirmed prostate-specific antigen (PSA) response was observed in 90% of subjects in the apalutamide arm and 2.2% of subjects in the placebo arm (p<0.0001); total response (confirmed and unconfirmed) was observed in 93% and 3.5% of subjects apalutamide- and placebo-treated subjects, respectively. Prostate-specific antigen progression was documented for 192 subjects in the apalutamide arm (24%) and 334 subjects in the placebo arm (83%). Treatment with apalutamide significantly decreased the risk of PSA progression by 94% compared with placebo (HR=0.064; 95% CI: 0.052, 0.080; p<0.0001). The median time to PSA progression was not estimable for the apalutamide arm and was 3.7 months in the placebo arm. The lower limit of the 95% CI for the median time to PSA progression for the apalutamide arm was 36.8 months. The Kaplan-Meier curves show separation favoring the apalutamide arm at approximately 4 months and widen from that point forward; this separation became greater over the course of time measured.

Treatment-emergent adverse events (AEs) were reported for 97% of subjects in the apalutamide arm and for 93% of subjects in the placebo arm. Grade 3-4 TEAEs were reported for 45% of subjects in the apalutamide arm and for 34% of subjects in the placebo arm; 11% of subjects discontinued treatment due to TEAEs in the apalutamide arm and 7% of subjects discontinued treatment due to TEAEs in the placebo arm. Serious adverse events (SAEs) were reported for 25% of subjects in the apalutamide arm and for 23% of subjects in the placebo arm. Treatment-emergent adverse events leading to death within 28 days of last dose were reported for 1.2% of subjects in the apalutamide arm and for 0.3% of subjects in the placebo arm.

The most frequently reported TEAEs (ie, occurring in >15% of subjects in either arm) were fatigue (30% apalutamide versus 21% placebo), hypertension (25% apalutamide versus 20% placebo), skin rash as the grouped term (24% apalutamide versus 5.5% placebo), diarrhea (20% apalutamide versus 15% placebo), nausea (18% apalutamide versus 16% placebo), weight decreased (16% apalutamide versus 6.3% placebo), arthralgia (16% apalutamide versus 7.5% placebo), and fall (16% apalutamide versus 9.0% placebo). After adjusting for exposure, the incidence of hypertension was similar in both arms and was not considered attributable to apalutamide; the incidence of diarrhea and nausea was higher in the placebo arm, and the incidence of fatigue, skin rash as the grouped term, weight decreased, arthralgia, and fall remained higher in the apalutamide arm.

There was a higher incidence of the AEs of special interest reported for the apalutamide arm (42%) compared with the placebo arm (18%). Adverse events (all grades) of special interest (apalutamide versus placebo) were skin rash (24% versus 5.5%), fall (16% versus 9.0%), fracture (12% versus 6.5%), seizure (0.2% versus 0.0%) and hypothyroidism (8.1% versus 2.0%). Most TEAEs of skin rash were Grade 1 or 2. More Grade 3 skin rashes were reported with apalutamide treatment (5.2% apalutamide arm versus 0.3% placebo arm). Two apalutamide treated subjects had an event of skin rash reported as an SAE. There were no reported Stevens-Johnson syndrome or toxic epidermal necrolysis cases. Skin rashes were manageable with dose interruption, dose reduction, topical, and systemic therapy. No TEAE of skin rash led to death. Most TEAEs of fracture and fall were of Grade 1 or 2 severity. The use of bone-sparing agents at study entry did not appear to be protective. Only 2 subjects in the apalutamide arm (1 subject Grade 1; 1 subject Grade 2) and no subjects in the placebo arm reported seizure, resulting in protocol-required discontinuation of study drug. All TEAEs of hypothyroidism were of Grade 1 or 2 severity. Hypothyroidism was detected since thyroid stimulating hormone (TSH) was regularly monitored in, and was managed with thyroid replacement therapy when medically indicated. Increases in TSH were at times associated with decreases in triiodothyronine (T3) or thyroxine (T4) or both. The mechanism for this observed TSH appeared to reflect increased metabolism of thyroid hormones due to increased glucuronosyltransferase activity by apalutamide (via activation of the nuclear pregnane X receptor).

### Safety in Healthy Subjects:

Based on the mechanism of action of apalutamide and after administration of the therapeutic dose of 240 mg, a transient increase was observed for testosterone and luteinizing hormone (LH) in healthy men, which normalized or returned to baseline during follow up. Reversible mild increases in TSH were observed in healthy men after single dose intake of apalutamide 240 mg. Sexual side effects (reduced libido Grade 1, loss of morning erection Grade 1) have occasionally been reported in healthy male subjects after single dose intake of apalutamide and recovered during follow-up.

### Overall Rationale for the Study:

This study was designed to determine the relative bioavailability of apalutamide administered orally as whole tablets and as a mixture of dispersed tablets in applesauce under fasted conditions. As a drug administration vehicle, applesauce can provide a convenient medium for the dispersion of apalutamide that avoids the need to crush tablets via mortar and pestle, tools that are not commonly available in a home care setting. This convenience is supported by studies that confirm the drug was found to be stable in applesauce for at least 6 hours. Additionally, applesauce can provide improved palatability and swallowability. Ingestion of 4 oz (120 mL) applesauce as well as 4 oz water in the cup to rinse is a convenient method to imitate the dosing of whole tablets with the standard 8 fl oz (240 mL) of water. The dispersion of the approved dose of 4×60 mg tablets in applesauce is intended to represent real world use for patients who have difficulty to swallow.

### Objectives and Hypothesis:

The primary objective was to determine the bioavailability of apalutamide tablets administered orally as dispersed tablets mixed in applesauce relative to whole tablets under fasting conditions in healthy male subjects.

The secondary objective was to assess the safety profile of apalutamide following single dose administration as whole tablets and as dispersed tablets mixed in applesauce.

The exploratory objective was to evaluate the palatability of apalutamide-applesauce mixture. This is an exploratory study to provide point estimation and no formal hypothesis was tested.

### Overview of Study Design

This was a randomized, open-label, balanced, single dose, two-treatment, two-period, two-sequence, crossover relative bioavailability study. 12 Healthy male subjects were randomly assigned in this study to one of the two treatment sequences based on a computer-generated randomization schedule prepared before the study by or under the supervision of the sponsor.

A dose of 240 mg (4×60 mg tablets) apalutamide was given in each treatment period under fasted conditions, as noted under the treatment sequence in Table E1, below.

TABLE E1
Treatment Sequence for Randomization Scheme

| Sequence | No. of | Period | Period |
|---|---|---|---|
| Number | Subjects | 1 | 2 |
| 1 | 6 | A | B |
| 2 | 6 | B | A |

Treatment A: (Reference treatment): 240 mg of apalutamide commercial formulation given as a single dose of 4×60 mg tablets swallowed whole under fasted conditions.

Treatment B (Test Treatment): 240 mg of apalutamide commercial formulation given as a single dose of 4×60 mg tablets as a dispersed mixture in applesauce under fasted conditions.

There was a screening phase (within 21 days before study drug administration in the first period); an open-label treatment phase consisting of 2 single-dose treatment periods; and end of study or early withdrawal assessments done upon completion of the 168-hour PK sampling on Day 8 of period 2 or upon early withdrawal. Successive study drug administrations were separated by a washout interval of at least 42 days and no more than 56 days between doses. The duration of participation in the study for an individual subject was approximately 84 days (including screening).

Subjects entered the study center in the morning on Day −1 and remained there until after the collection of the 72-hour PK sample on Day 4 if the investigator considers the subject ready for discharge. On Day 1, a single dose of the appropriate study drug preparation was administered to each subject in the morning followed by sequential collection of blood samples over 168 hours (Day 8) for the determination of apalutamide plasma concentrations. Subjects returned daily to the study center for PK sampling from Day 5 to Day 8. Subject safety was monitored throughout the study. Details on the timing of the apalutamide administrations, PK sampling time points and safety assessments are given in the Time and Events Schedule.

In order to assess the palatability of the apalutamide dispersed in applesauce (Treatment B), a taste questionnaire (shown in FIG. 2) was performed within 30 min after intake of Treatment B.

The questionnaire consisted of a visual analogue scale to rate 3 items (sweetness, bitterness, and smell) as well as overall acceptability (not acceptable or acceptable), as shown below.

### Study Design Rationale:

A single-dose, 2-way crossover design to evaluate the bioavailability of apalutamide tablets administered orally as dispersed tablets mixed in applesauce in comparison to whole tablets was chosen for this study, and subjects fasted for 10 hours before study drug administration in accordance with regulatory guidelines (FDA Guidance 2003). Single-dose studies are considered more sensitive than multiple-dose studies in addressing the rate and extent of drug absorption. The crossover design permits intrasubject comparisons since each subject was his own control, so that a smaller number of subjects is required compared to parallel group design.

As the half-life of apalutamide in human is long (˜7 days), a truncated AUC was used to evaluate extent of absorption as per healthcare agency guidance. A 168-hour period was selected as historical data has indicated that AUC_{0-168 h} is a good predictor for AUC_{∞} of apalutamide. Additionally, AUC_{0-72 h} was also assessed to meet the regulatory needs of various regions. Also taking the long half-life into account, a 6-8 week washout period was planned to ensure no carry-over effects.

Healthy subjects are commonly selected for PK studies as they allow for assessments in the absence of confounding factors such as comorbid conditions and concomitant medications. In this study, only male subjects were enrolled as the target patient population for clinical use of apalutamide consists of male subjects with CRPC. Previous studies in healthy subjects have demonstrated that single doses of apalutamide up to 240 mg are well tolerated.

### Blinding, Control, Study Phase/Periods, Treatment Groups:

Randomization was used to minimize bias in the assignment of subjects to treatment sequence groups, to increase the likelihood that known and unknown subject attributes (eg, demographic and baseline characteristics) are evenly balanced across treatment sequence groups, and to enhance the validity of statistical comparisons across treatment groups.

### Subject Population

### General Considerations:

12 Healthy subjects were included in the study population to ensure that at least 10 subjects complete all required assessments (see Section Error! Reference source not found., Completion).

The inclusion and exclusion criteria for enrolling subjects in this study were described in the following sections. If there was a question about the inclusion or exclusion criteria below, the investigator was instructed to consult with the appropriate sponsor representative and resolve any issues before enrolling a subject in the study. Waivers were not allowed. Inclusion Criteria:

Subjects needed to satisfy the following criteria to be enrolled in the study:
1. a healthy male.
2. 18 to 55 years of age, inclusive
3. must sign an informed consent form (ICF) indicating they understand the purpose of, and procedures required for, the study and is willing to participate in the study.
4. must agree to use an adequate contraception method as deemed appropriate by the investigator and specified in Prohibitions and Restrictions Section, always use a condom during sexual intercourse.
5. must agree not to donate sperm for the purpose of reproduction during the study and for a minimum of 3 months after receiving the last dose of study drug.
6. body mass index (BMI; weight [kg]/height² [m]²) between 18 and 30 kg/m² (inclusive), and body weight not less than 50 kg.
7. blood pressure (after the subject is sitting for at least 5 minutes) between 90 and 140 mmHg systolic, inclusive, and no higher than 90 mmHg diastolic. If blood pressure is out of range, up to 2 repeated assessments are permitted.
8. a 12-lead ECG at screening consistent with normal cardiac conduction and function, including:
   Sinus rhythm
   Pulse rate between 40 and 100 beats per minute (bpm)
   QTc interval ≤450 milliseconds (corrected Fridericia; QTcF)
   QRS interval of <120 milliseconds
   PR interval <210 milliseconds
   Morphology consistent with healthy cardiac conduction and function
9. nonsmoker within the last 2 months (calculated from the first dosing).

### Exclusion Criteria:

Any potential subjects who met any of the following criteria was excluded from participating in the study.
1. history of or current clinically significant medical illness including (but not limited to) cardiac arrhythmias or other cardiac disease, hematologic disease, coagulation disorders (including any abnormal bleeding or blood dyscrasias), lipid abnormalities, significant pulmonary disease, including bronchospastic respiratory disease, diabetes mellitus, hepatic or renal insufficiency, thyroid disease, neurologic or psychiatric disease, infection, inflammatory bowel disease, gall bladder or biliary tract disease (including gall stones) or any other illness that the investigator considers should exclude the subject or that could interfere with the interpretation of the study results.
2. clinically significant abnormal values for hematology or clinical chemistry at screening or at admission to the study center as deemed appropriate by the investigator.
3. clinically significant abnormal physical examination, vital signs, or 12-lead electrocardiogram (ECG) at screening or at admission to the study center as deemed appropriate by the investigator.
4. has a history of significant multiple and/or severe allergies, or has had an anaphylactic reaction or significant intolerability to prescription or non-prescription drugs or food including to excipients of apalutamide or to apple or applesauce.
5. history of malignancy within 5 years before screening (exceptions are squamous and basal cell carcinomas of the skin and carcinoma in situ of the cervix, or malignancy, which is considered cured with minimal risk of recurrence).
6. use of any prescription or nonprescription medication (including vitamins and herbal supplements), except for paracetamol, within 14 days before the first dose of the study drug is scheduled until completion of the study.
7. history of, or a reason to believe a subject has a history of drug or alcohol abuse within the past 5 years.
8. donated blood or blood products or had substantial loss of blood (more than 500 mL) within 3 months before the first administration of study drug or intention to donate blood or blood products during the study.
9. received an experimental drug (including investigational vaccines) or used an experimental medical device within 1 month or within a period less than 10 times the drug's half-life, whichever is longer, before the first dose of the study drug is scheduled.
10. history of stomach or intestinal surgery or resection (including prior cholecystectomy) that would potentially alter absorption or excretion of orally administered drugs (appendectomy and hernia repair will be allowed) or unable to swallow solid, oral dosage forms whole with the aid of water (participants may not chew, divide, or crush the study drug).
11. plans to father a child while enrolled in the study or within 12 weeks after the last dose of study drug.
12. any condition for which, in the opinion of the investigator, participation would not be in the best interest of the subject (eg, compromise the well-being) or that could prevent, limit, or confound the protocol-specified assessments.
13. tests positive for hepatitis A antibody IgM, hepatitis B surface antigen (HBsAg), or hepatitis C antibody (anti-HCV) at Screening.
14. history of human immunodeficiency virus (HIV) antibody positive, or tests positive for HIV at screening.
15. history of smoking or use of nicotine-containing substances within the previous 2 months, as determined by medical history or subject's verbal report.
16. preplanned surgery or procedures that would interfere with the conduct of the study.
17. employee of the investigator or study center, with direct involvement in the proposed study or other studies under the direction of that investigator or study center, as well as family members of the employees or the investigator.
18. presence of sexual dysfunction (eg, abnormal libido, erectile dysfunction) or any medical condition that would affect sexual function.
19. screening serum testosterone level of <200 ng/dL.
20. screening TSH level >upper limit of normal.
21. positive test for alcohol or drugs of abuse, such as cannabinoids, opiates, cocaine, amphetamines, benzodiazepines, or barbiturates at screening and Day −1.
22. history of seizure or condition that may predispose to seizure (eg, transient ischemic attack, stroke, brain arteriovenous malformation, neoplasm in brain or meninges, other).
   NOTE: Investigators were to ensure that all study enrollment criteria were met at screening. If a subject's clinical status changed (including any available laboratory results or receipt of additional medical records) after screening but before the first dose of study drug was given such that he or she no longer met all eligibility criteria, then the subject was to be excluded from participation in the study. The Source Documentation Section describes the required documentation to support meeting the enrollment criteria. Retesting of abnormal lab values that may lead to exclusion was allowed once. Retesting to replace lost samples or broken tubes was permitted. Retesting was to take place during an unscheduled visit during screening.

### Prohibitions and Restrictions:

Potential subjects must be willing to adhere to the following prohibitions and restrictions during the course of the study to be eligible for participation:
1. agree to follow all requirements that must be met during the study as noted in the Inclusion and Exclusion Criteria (eg, contraceptive requirements, etc).
2. refer to Pre-study and Concomitant Therapy Section for details regarding prohibited and restricted therapy during the study.
3. may not consume food or beverages containing grapefruit juice and Seville oranges from 72 hours before Day 1, until after the last PK sample is collected at 168 hours on Day 8 in each treatment period.
4. may not consume alcohol from 24 hours before each PK sample collection day, until after the last PK sample is collected at 168 hours on Day 8 in each treatment period.
5. must refrain from the use of any methylxanthine-containing products, (eg, chocolate bars or beverages, energy drinks if it contains methylxanthine, coffee, teas, or colas) from 48 hours before administration of study drug and during residence in the study center, and also must avoid excessive use of caffeine (ie, no more than approximately 500 mg/day, as contained in 5 cups of tea or coffee or 8 cans of cola) from screening to after the last PK sample is collected at 168 hours on Day 8 of Treatment Period 1 and from Day −1 until after the last PK sample is collected at 168 hours on Day 8 of Treatment Period 2.
6. may not consume food containing poppy seeds during the study starting 72 hours before screening or 72 hours before admission to study center in each treatment period (in order to avoid false positive urine drug test for codeine).
7. subjects must consume standard institutional meals during residence in the study center. Excessive food consumption will not be permitted until after the last PK sample is collected at 168 hours on Day 8 in each treatment period.
8. must refrain from the use of nicotine-containing substances, including tobacco products (eg, cigarettes, e-cigarettes, cigars, chewing tobacco, gum, or patch) throughout the study.
9. if a subject has had a recent febrile illness (>38° C.) within 3 days of the scheduled start of study drug intake, the start of study drug intake should be postponed until the body temperature is normal for at least 72 hours.
10. subjects will be advised not to donate blood for at least 2 months after completion of the study or to participate in an investigational drug study for at least 3 months after receiving the last dose of study drug.
11. must refrain from jogging and strenuous exercise of all types during residence in the study center and within 48 hours before admission to the study center.
12. must remain at study center from at least 10 hours before study drug administration (Day −1) until 72 hours after study drug administration (Day 4) and agree to return to study center for daily blood sampling until 168 hours after study drug administration (Day 8) in each treatment period.
13. must agree to always use a condom during sexual intercourse (also in case of prior vasectomy) or remain abstinent during the study and for 3 months after last study drug administration. In case of sexual activity with a woman of childbearing potential, a condom is required along with another effective contraceptive method (hormonal contraception [pill, patch, injection, implant], intrauterine device, intrauterine hormone-releasing system, tubal ligation, or status post hysterectomy/bilateral ovariectomy or salpingectomy) for the duration of the study and for 3 months after the last study drug administration.

Further instructions for food intake or restrictions relating to the timing of study drug administration were provided in the Dosage and Administration Section.

### Subject Completion:

### Completion:

A subject was considered to have completed the study if he completed all required assessments up to Day 8 of Period 2 of the open-label treatment phase.

Subjects who prematurely discontinued study treatment for any reason before completion of the study on Day 8 of Period 2 were not be considered to have completed the study.

Discontinuation of Study Treatment/Withdrawal from the Study:

A subject's study treatment was discontinued if the investigator believed that for safety reasons (eg, adverse event) it was in the best interest of the subject to stop treatment.

If a subject discontinued study treatment before the end of treatment, planned safety assessments were still obtained according to the end-of-study visit in the TIME AND EVENTS SCHEDULE, shown in FIG. 1.

### Withdrawal From the Study:

A subject was to be withdrawn from the study for any of the following reasons:
lost to follow-up
withdrawal of consent
subject is not in compliance with requirements of the study, including prohibitions and restrictions

If a subject was lost to follow up, every reasonable effort was made by the study center personnel to contact the subject and determine the reason for discontinuation. The measures taken to follow up must be documented.

When a subject withdrew before completing the study, the reason for withdrawal was to be documented on the case report form (CRF) and in the source document. Study drug assigned to the withdrawn subject was not be assigned to another subject. If the number of PK evaluable subjects (a PK evaluable subject was defined by having sufficient and interpretable PK assessments to calculate at least 1 noncompartmental PK parameter) who complete the study decreased to less than 10, additional subjects could be enrolled for replacement (see details Section Error! Reference source not found.). These replacement subjects were then assigned to the same treatment sequence as the subjects they are replacing.

### Study Treatment

### Randomization and Blinding:

Randomization was used to avoid bias in the assignment of subjects to treatment sequence groups, to increase the likelihood that known and unknown subject attributes (eg, demographic and baseline characteristics) are evenly balanced across treatment sequence groups, and to enhance the validity of statistical comparisons across treatments.

Subjects were assigned to 1 of 2 treatment sequence groups based on a computer-generated randomization schedule prepared prior to the first administration of study drug, by the sponsor by or by a designee authorized by the sponsor. The randomization was balanced by using randomly permuted blocks.

Assignment to treatment sequence groups occurred before the subject received the study drug.

Blinding of treatment was not performed as this was an open-label study. Blinding was not used because the primary objective, the assessment of specified pharmacokinetic parameters, was not subject to bias from the participants or observers.

### Dosage and Administration:

Before each study drug administration in each treatment period, subjects fasted from food for at least 10 hours. Noncarbonated water was allowed up to 1 hour before study drug administration.

All study drug was taken in the morning on Day 1 of each treatment period (see Section Error! Reference source not found.). Study drug was taken with 240 mL of noncarbonated water (Treatment A) or mixed with an approximate volume of 4 oz or 120 mL of applesauce supplemented by a container rinse of 120 mL of noncarbonated water (Treatment B). For Treatment B, the study drug-applesauce mixture was prepared by a pharmaceutical technician or pharmacist. A general guideline for the preparation of apalutamide tablets in applesauce (Treatment B) was as provided Table E2, below. An additional 50 mL of water for either treatments was allowed if necessary. Study drug of Treatment B was ingested within 5 minutes. Treatment A was swallowed whole and not chewed, divided, dissolved, or crushed. The exact dates and times of each study drug administration were documented in the source and transcribed in the CRF. For each subject, all doses were administered at approximately the same time of day.

Subjects continued fasting until 4 hours after study drug administration. At approximately 1 hour after dosing (but not earlier) all subjects drank 1 glass (approximately 240 mL) of water; drinking of water was allowed from then onwards. A standardized lunch was served on Day 1 of each period for all subjects after collection of the 4-hour PK blood sample. Standard meals and snacks were provided while the subjects are at the study center.

### Compliance:

Study drug was administered by site personnel to assure compliance with study requirements. The date and time of each study drug administration was recorded in the CRF.

### STUDY DRUG INFORMATION Stopped Here

### Physical Description of the Study Drug:

Apalutamide supplied for this study was formulated as a 60-mg tablet. The 60-mg apalutamide tablet (G023) for all treatments contains 60 mg of apalutamide as a SDP in hydroxypropyl methylcellulose-acetate succinate (HPMC-AS) polymer, in a 1/3 ratio (API/polymer). This oral coated tablet also contains the following inactive ingredients: colloidal anhydrous silica, croscarmellose sodium, microcrystalline cellulose, silicified microcrystalline cellulose, magnesium stearate and coating powder green OPADRY II. The tablet core weight is 700 mg.

### Packaging:

Apalutamide tablets were packaged in high-density polyethylene (HDPE) bottles (120 tablets) with child-resistant closures (CRC) and tamper-proof heat induction seals.

### Labeling:

Study drug labels contained information to meet the applicable regulatory requirements.

### Preparation, Handling and Storage

All study drug was stored at controlled temperatures ranging from 15° C. to 30° C. and kept in the original container to protect it from light.

The pharmacy manual/study center investigational product and procedures manual provided additional guidance on study drug preparation, handling, and storage. A general guideline for the preparation of dispersed apalutamide tablets in applesauce (Treatment B) is provided in Table E2, below.

TABLE E2
General Guidelines for Preparing Dispersed
Apalutamide Tablets in Applesauce (Treatment B)

| | |
|---|---|
| 1. | To ensure consistency, only trained pharmacists or pharmaceutical technicians are |
| | responsible for the procedures. |
| 2. | Tablets should be dispersed within 120 minutes before administration. |
| 3. | Four whole intact tablets should be placed in 4 oz or 120 mL of applesauce and |
| | gently stirred for 10 seconds. |
| 4. | After 15 minutes, the content should be stirred for 10 seconds. |
| 5. | After an additional 15 minutes, the content should be stirred for at least 10 seconds |
| | until a uniform mixture is formed (for oral administration in Treatment B). |
| 6. | After administration, the container of the drug product-applesauce mixture should |
| | be rinsed one time with 120 mL of non-carbonated water to ensure administration |
| | of the entire dose. |
| 7. | Please refer to the pharmacy manual for complete details of the preparation procedures. |

### Drug Accountability:

The investigator was responsible for ensuring that all study drug received at the site was inventoried and accounted for throughout the study. The study drug administered to the subject was documented on the drug accountability form. All study drug was stored and disposed of according to the sponsor's instructions. Study-site personnel must not combine contents of the study drug containers.

Study drug was dispensed under the supervision of the investigator or a qualified member of the study-site personnel, or by a hospital/clinic pharmacist or pharmacist technician. Study drug was supplied only to subjects participating in the study. Study drug could not be relabeled or reassigned for use by other subjects. The investigator agreed neither to dispense the study drug from, nor store it at, any site other than the study centers agreed upon with the sponsor.

### Pre-Study and Concomitant Therapy:

Pre-study therapies administered up to 30 days before first dose of study drug administration were recorded at screening.

Concomitant therapies were recorded throughout the study beginning with start of the first dose of study drug administration up to Day 8 of Period 2 or up to the End-of-Study visit.

Throughout the study, prescription or nonprescription medication (including vitamins and herbal supplements) other than the study drug were prohibited, except for paracetamol. The use of paracetamol was allowed until 3 days before the first study drug administration. Throughout the study, a maximum of three 500-mg doses per day paracetamol, and no more than 3 g per week, were allowed for the treatment of headache or other pain. If paracetamol was used, the dose and dosage regimen and the reason for use must be recorded in the case report form (CRF).

All medication were discontinued at least 14 days before the first intake of study drug (Day 1 of Period 1), except for paracetamol. Subjects could not use any medication other than the study drug up to 8 days after the last intake of study drug, except for paracetamol. Subjects could not use any systemic herbal medications or dietary supplements including products containing Hypericum perforatum (St. John's Wort) from 14 days before the first intake of study drug and up to 8 days after the last intake of study drug. The sponsor must be notified immediately if prohibited therapies were administered.

All medications taken by a subject (prescription or nonprescription, including vaccines, vitamins, and herbal supplements) that were not the study drug were documented in the concomitant therapy section of the CRF. This included medications taken before and during the study. Modification of an effective preexisting therapy should not be made for the explicit purpose of entering a subject into the study. Nonpharmacologic therapies such as electrical stimulation, acupuncture, special diets, and exercise regimens were also recorded in the CRF.

### Study Evaluations

### Study Procedures:

The Error! Reference source not found. in FIG. 1, summarizes the frequency and timing of PK and safety measurements applicable to this study.

If multiple assessments were scheduled for the same time point, it was recommended that procedures be performed in the following sequence: 12-lead ECGs, vital signs, PK blood sample collection, clinical laboratory tests, weight and physical examination. Blood collections for pharmacokinetic assessments were kept as close to the specified time as possible. Other measurements could be done earlier than specified time points, if needed. Actual dates and times of assessments were recorded in the source documents and CRF.

Table E3 below, lists the volume of blood that was collected from each subject and each type of assessment.

TABLE E3
Volume of Blood to be Collected From Each Subject

| | | | No. of | Total | |
|---|---|---|---|---|---|
| | Volume per | | Samples | Volume | |
| | Sample | | per | of Blood | |
| Type of Sample | (mL) | | Subject | (mL)^{a} | |
| Safety (including screening and | | | | | |
| posttreatment assessments) | | | | | |
| Hematology | 4 | mL | 8 | 32 | mL |
| Serum chemistry, testosterone, | 8.5 | mL | 8 | 68 | mL |
| TSH & serology | | | | | |
| Pharmacokinetic samples | 2 | mL | 38 | 76 | mL |
| Loss by use of indwelling | 0.5 | mL | 38 | 19 | mL |

| intravenous cannula | | |
|---|---|---|
| Total | 195 | mL |

^{a}Calculated as number of samples multiplied by amount of blood per sample.
Note:
An indwelling intravenous cannula may be used for blood sample collection.

The total blood volume to be collected according to the protocol was considered to be within the normal range allowed for this subject population over this time frame. Repeat or unscheduled samples could be taken for safety reasons, but the maximum amount of blood drawn in this study was not to exceed 250 mL.

If blood samples were collected via an indwelling cannula, an appropriate amount (ie, 0.5 mL) of fluid, slightly greater than the dead space volume of the lock, was removed from the cannula and discarded before each blood sample was taken. After blood sample collection, the cannula was flushed with 0.9% pharmaceutical grade for injection sodium chloride or sodium heparin of 10 U/mL and charged with a volume equal to the dead space volume of the lock.

A taste questionnaire, shown in FIG. 2, was performed within 30 min after intake of Treatment B.

### Pharmacokinetic Evaluations

### Sample Collection and Handling:

Blood samples (2 mL each) for determination of apalutamide plasma concentrations were collected at the time points indicated in the Error! Reference source not found. (See FIG. 1). During the study, the nominal sample collection times could be changed by the sponsor with clear communication to the investigator, but the total blood volume was not to exceed 250 mL. The exact dates and times of blood sample collection were recorded in the CRF.

The laboratory manual contained further information regarding the collection, handling, labeling, and shipment of plasma samples.

### Analytic Procedures:

Plasma samples were analyzed to determine concentrations of apalutamide using a validated, specific, and sensitive liquid chromatography/mass spectrometry/mass spectrometry (LC-MS/MS) method by or under the supervision of the sponsor's Department of Bioanalysis.

If required, some plasma samples were analyzed to document the presence of circulating metabolites using a qualified research method. In addition, plasma PK samples were stored for future analysis of the metabolite profile.

Pharmacokinetic Parameters:

Pharmacokinetic analysis was performed by or under the responsibility of the sponsor. The following PK parameters of apalutamide were determined:
Cₘₐₓ maximum plasma concentration
tₘₐₓ time to reach the maximum plasma concentration
AUC_{0-72 h} area under the plasma concentration-time curve from time 0 to 72 hours
AUC_{0-168 h} area under the plasma concentration-time curve from time 0 to 168 hours

Additional PK parameters could be included if deemed appropriate.

Additional details of the PK analysis, including data handling rules and software used to perform the PK analysis were provided in the Clinical Pharmacology Analysis Plan.

### Safety Evaluations:

Safety was evaluated throughout the study by means of AEs, physical examination, vital signs, ECG, and laboratory safety (hematology and serum chemistry) (see the Error! Reference source not found. in FIG. 1 for details).

Any clinically relevant changes occurring during the study were recorded on the Adverse Event section of the CRF.

Any clinically significant abnormalities persisting at the end of the study/early withdrawal were followed by the investigator until resolution or until a clinically stable condition is reached.

The study included the following evaluations of safety according to the time points provided in the Time and Events Schedule in FIG. 1:

Adverse Events:

Adverse events were reported by the subject (or, when appropriate, by a caregiver, surrogate, or the subject's legally acceptable representative) for the duration of the study (see Adverse Event Reporting Section).

### Clinical Laboratory Tests:

Blood samples for serum chemistry and hematology were taken for evaluation of laboratory safety parameters. Blood samples for laboratory safety tests were taken fasted for at least 10 hours. The investigator reviewed the laboratory report, documented this review, and recorded any clinically relevant changes occurring during the study in the adverse event section of the CRF. The following tests were performed by the local laboratory:

### Hematology Panel

hemoglobin-platelet count
hematocrit
red blood cell (RBC) count
white blood cell (WBC) count with differential
Note: A WBC evaluation included any abnormal cells, which were reported by the laboratory. A RBC evaluation included abnormalities in the RBC count, RBC parameters, or RBC morphology, which were reported by the laboratory. In addition, any other abnormal cells in a blood smear were also reported.

Serum Chemistry Panel

| | |
|---|---|
| sodium | creatine phosphokinase (CPK) |
| potassium | lactic acid dehydrogenase (LDH) |
| chloride | uric acid |
| bicarbonate | calcium |
| ureum | phosphate |
| creatinine | albumin |
| glucose | total protein |
| aspartate aminotransferase (AST) | total cholesterol |
| alanine aminotransferase (ALT) | triglycerides |
| gamma-glutamyltransferase (GGT) | testosterone* |
| total bilirubin | TSH* |
| alkaline phosphatase | |

*At screening only

Other Clinical Laboratory Tests:
Serology (HIV antibody, HBsAg, Hep A IgM and hepatitis C virus antibody) at screening
Urine Drug Screen (cannabinoids, opiates, cocaine, amphetamines, benzodiazepines, or barbiturates) at screening and Day −1
Alcohol test at screening and on Day −1
Cotinine test at screening and on Day −1

### Electrocardiogram:

During the collection of ECGs, subjects were in a quiet setting without distractions (eg, television, cell phones). Subjects rested in a supine position for at least 5 minutes before ECG collection and refrained from talking or moving arms or legs. If blood sampling or vital sign measurement was scheduled for the same time point as ECG recording, the procedures were performed in the following order: ECG(s), vital signs, blood draw.

### Vital Signs (Blood Pressure, Pulse, Respiratory Rate and Body Temperature):

Blood pressure and pulse measurements were assessed after the subject sat for at least 5 minutes with a completely automated device. Manual techniques were used only if an automated device was not available.

Blood pressure and pulse/heart rate measurements were preceded by at least 5 minutes of rest in a quiet setting without distractions (eg, television, cell phones).

### Physical Examination:

Physical examinations including height and body weight were conducted at times indicated in the Error! Reference source not found. in FIG. 1.

Full physical exam was performed at screening and at end of study, otherwise exams were abbreviated (minimum of cardiovascular, respiratory, and gastrointestinal exam with option to include additional body systems as indicated by adverse events/symptoms).

### Taste Evaluation:

In order to assess the palatability of the apalutamide dispersed in applesauce (Treatment B), a taste questionnaire (See FIG. 2) was performed within 30 min after intake of Treatment B.

The questionnaire consisted of a visual analogue scale to rate 3 items (sweetness, bitterness, and smell) as well as overall acceptability (not acceptable or acceptable). Each subject completed the questionnaire individually and privately during this assessment.

The results of the taste questionnaire were transcribed into the electronic CRF (eCRF) by a member of the study-site personnel.

### Statistical Methods:

Statistical analysis were done by the sponsor or under the supervision of the sponsor. A general description of the statistical methods to be used to analyze the efficacy and safety data is outlined below. Specific details were provided in the Statistical Analysis Plan.

### Sample Size Determination:

More recent studies indicated the intra-subject coefficient of variation (CV) for Cₘₐₓ and AUCs of apalutamide ranged 11%-12% and 4%-7%, respectively.

Applying a conservative assumption for intra-subject CV of 12% for Cₘₐₓ and 7% for AUCs respectively, a sample size of 10 subjects in the current study was determined to be sufficient for the point estimates of the geometric mean ratios of Cₘₐₓ and AUCs to fall within (91%, 110%) and (95%, 105%) of the true value respectively, with 90% confidence.

Assuming a dropout rate of 17%, approximately 12 subjects were randomized to ensure at least 10 PK evaluable (a PK evaluable subject is defined by having sufficient and interpretable PK assessments to calculate at least 1 noncompartmental PK parameter) subjects complete the study. If the number of PK evaluable subjects who complete the study fell to less than 10, additional subjects could be enrolled for replacement by assigning them to the same treatment sequence as the subjects being replaced. Replacement subjects were to start with Period 1. Subjects who prematurely discontinued from the study due to drug related AEs or AEs which occur in relation to study procedures were not to be replaced.

### Initial Subject Characteristics:

For all subjects who received at least one dose of study drug, descriptive statistics (mean, standard deviation, median, minimum, and maximum) were performed for age, BMI, weight, and height. Sex and race were listed and tabulated.

### Pharmacokinetic Analyses:

Pharmacokinetic and statistical analysis were done using Phoenix™ WinNonlin® (Tripos L.P.). Noncompartmental analysis was applied for the pharmacokinetic analysis. Furthermore, Microsoft Excel® (Microsoft, Redmond, Wash., United States), and SAS (SAS Institute Inc., Cary, N.C., US) were used.

For each treatment, descriptive statistics were calculated for plasma concentrations of apalutamide, as applicable, at each applicable time point specified, and for the derived plasma pharmacokinetic parameters. Statistics included sample size (n), mean, SD, % CV, geometric mean, median, minimum, and maximum.

For each subject and per treatment, apalutamide plasma concentration-time data, as applicable, may be graphically presented. Similarly, graphs of the mean apalutamide plasma concentration-time profiles and overlay graphs with combined individual apalutamide plasma concentration-time profiles may be produced. Pharmacokinetic parameters may be subjected to additional graphical analyses.

Graphical representations of the results may include, but are not limited to, the following graphs:

Log-linear and linear-linear plasma concentration-time profiles for each individual

Log-linear and linear-linear plasma concentration-time profiles for the mean values per treatment.

The PK population will include all subjects who have sufficient and interpretable concentration-time data.

Data was listed for all subjects with available study drug plasma concentrations per treatment. All concentrations below lower limit of quantification (LLOQ) or missing data were labeled as such in the concentration data listings. Concentrations below LLOQ were treated as zero in the summary statistics and for the calculation of PK parameters. All subjects and samples excluded from the analysis were clearly documented in the CSR.

Factors that may influence the study drug plasma concentrations (eg, vomiting, concomitant medication, fever, high predose concentration) were checked. If an influencing factor was present, a decision was made by the responsible pharmacokineticist, whether to include or exclude the specific sample or subject.

Reasons for exclusion of a subject or a sample from the analysis included, but were not limited, to the following:
Predose plasma concentrations higher than 5% of Cₘₐₓ
Vomiting occurred at or before 2 times median tₘₐₓ after study drug administration for immediate-release products
Noncompliance with study procedures affecting PK (eg, concomitant medication)

The primary objective of the statistical analysis was to determine the relative bioavailability of Treatment B with respect to the reference Treatment A. The primary parameters of interest for the statistical analysis were Cₘₐₓ, AUC_{0-72 h} and AUC_{0-168 h}. If one of the PK parameters could not be determined for a given subject in 1 or more periods, the subject's data was not included in the statistical analysis of that particular PK parameter. The analysis was performed on log-transformed PK parameters.

A mixed effect model that includes treatment, period, and treatment sequence as fixed effects, and subject as a random effect, was used to estimate the least squares means and intrasubject variance. Using these estimated least squares means and intrasubject variance, the point estimate and 90% CIs for the difference in means on a log scale between test and reference were constructed. The limits of the CIs was retransformed using antilogarithms to obtain 90% CIs for the geometric mean ratios of Cₘₐₓ and AUC_{0-168 h} of the test to reference formulation. A similar analysis was conducted for AUC_{0-72 h} for supplemental purpose.

Additional statistical analysis could be performed if deemed appropriate.

### Safety Analyses:

All subjects who were randomly assigned to treatment and received at least one dose of the study drug were included in the safety analysis.

Baseline for all laboratory evaluations, vital signs, and ECG measurements were defined as the last evaluation done before the first study drug administration.

### Adverse Events:

The verbatim terms used in the CRF by investigators to identify adverse events was coded using the Medical Dictionary for Regulatory Activities (MedDRA). All reported adverse events with onset during the treatment phase (ie, treatment-emergent adverse events, and adverse events that have worsened since baseline) were included in the analysis. For each adverse event, the percentage of subjects who experience at least 1 occurrence of the given event was summarized by treatment group.

Summaries, listings, datasets, or subject narratives were to be provided, as appropriate, for those subjects who died, who discontinued treatment due to an adverse event, or who experienced a severe or a serious adverse event.

### Clinical Laboratory Tests:

Laboratory data were summarized by type of laboratory test. Reference ranges and markedly abnormal results (specified in the Statistical Analysis Plan) were used in the summary of laboratory data. Descriptive statistics were calculated for each laboratory analyte at baseline and at each scheduled time point. Changes from baseline results were presented in pre-versus posttreatment cross-tabulations (with classes for below, within, and above normal ranges). A listing of subjects with any laboratory results outside the reference ranges was provided. A listing of subjects with any markedly abnormal laboratory results was also provided.

Parameters with predefined National Cancer Institute Common Terminology Criteria for Adverse Events (NCI-CTCAE v5.0) toxicity grades were summarized. Change from baseline to the worst adverse event grade experienced by the subject during the study were provided as shift tables.

### Electrocardiogram (ECG):

The effects on cardiovascular variables was evaluated by means of descriptive statistics and frequency tabulations. These tables included observed values and changes from baseline values. Screening ECG was used as baseline to allow detection of clinically relevant changes in individuals. Baseline for all ECG evaluations was defined as the last evaluation done before study drug administration in Treatment Period 1.

Electrocardiogram data were summarized by ECG parameter. Descriptive statistics were calculated at baseline and for observed values and changes from baseline at each scheduled time point. Frequency tabulations of the abnormalities were made.

The ECG variables that were analyzed are heart rate, PR interval, QRS interval, QT interval, and corrected QT (QTc) interval using the following correction methods: QT corrected according to Bazett's formula (QTcB), QT corrected according to Fridericia's formula (QTcF)^{Error! Reference source not found. Error! Reference source not found}.

Descriptive statistics of QTc intervals and changes from baseline were summarized at each scheduled time point. The percentage of subjects with QTc interval >450 milliseconds, >480 milliseconds, or >500 milliseconds were summarized, as were the percentage of subjects with QTc interval increases from baseline >30 milliseconds or >60 milliseconds.

All clinically relevant abnormalities in ECG waveform that were changes from the baseline readings were reported.

### Vital Signs:

Descriptive statistics of temperature, pulse, respiratory rate, and blood pressure (systolic and diastolic) values and changes from baseline were summarized at each scheduled time point. The percentage of subjects with values beyond clinically important limits were summarized.

### Physical Examination:

Results of physical examinations (abnormalities) were listed.

### Taste Evaluation Analyses:

Questionnaire data (sweetness, bitterness, smell, and overall acceptability) was summarized using descriptive statistics.

### Adverse Event Reporting:

Timely, accurate, and complete reporting and analysis of safety information from clinical studies are crucial for the protection of subjects, investigators, and the sponsor, and are mandated by regulatory agencies worldwide. The sponsor has established Standard Operating Procedures in conformity with regulatory requirements worldwide to ensure appropriate reporting of safety information; all clinical studies conducted by the sponsor or its affiliates were conducted in accordance with those procedures.

### Method of Detection Adverse Events and Serious Adverse Events:

Care was taken not to introduce bias when detecting adverse events or serious adverse events. Open-ended and nonleading verbal questioning of the subject was the preferred method to inquire about adverse event occurrence.

### DEFINTIONS: Adverse Event Definitions and Classifications:

### Adverse Event:

An adverse event is any untoward medical occurrence in a clinical study subject administered a medicinal (investigational or non-investigational) product. An adverse event does not necessarily have a causal relationship with the treatment. An adverse event can therefore be any unfavorable and unintended sign (including an abnormal finding), symptom, or disease temporally associated with the use of a medicinal (investigational or non-investigational) product, whether or not related to that medicinal (investigational or non-investigational) product. (Definition per International Council for Harmonisation of Technical Requirements for Pharmaceuticals for Human Use [ICH]).

This includes any occurrence that is new in onset or aggravated in severity or frequency from the baseline condition, or abnormal results of diagnostic procedures, including laboratory test abnormalities.

Note: The sponsor collected adverse events starting with the signing of the ICF (refer to the All Adverse Events Section, for time of last adverse event recording).

### Serious Adverse Event:

A serious adverse event based on ICH is any untoward medical occurrence that at any dose:
Results in death
Is life-threatening (The subject was at risk of death at the time of the event. It does not refer to an event that hypothetically might have caused death if it were more severe.)
Requires inpatient hospitalization or prolongation of existing hospitalization
Results in persistent or significant disability/incapacity
Is a congenital anomaly/birth defect
Is a suspected transmission of any infectious agent via a medicinal product
Is medically important*

*Medical and scientific judgment should be exercised in deciding whether expedited reporting is also appropriate in other situations, such as important medical events that may not be immediately life threatening or result in death or hospitalization but may jeopardize the subject or may require intervention to prevent one of the other outcomes listed in the definition above. These should usually be considered serious.

### Unlisted (Unexpected) Adverse Event/Reference Safety Information:

An adverse event is considered unlisted if the nature or severity is not consistent with the applicable product reference safety information. For apalutamide, with a marketing authorization in the United States and Canada, the expectedness of an adverse event was determined.

Adverse Event Associated with the Use of the Drug:

An adverse event was considered associated with the use of the drug if the attribution is possible, probable, or very likely by the definitions listed in the Attribution Definitions Section.

### Attribution Definitions:

Not Related: An adverse event that is not related to the use of the drug.

Doubtful: An adverse event for which an alternative explanation is more likely, eg, concomitant drug(s), concomitant disease(s), or the relationship in time suggests that a causal relationship is unlikely.

Possible: An adverse event that might be due to the use of the drug. An alternative explanation, eg, concomitant drug(s), concomitant disease(s), is inconclusive. The relationship in time is reasonable; therefore, the causal relationship cannot be excluded.

Probably: An adverse event that might be due to the use of the drug. The relationship in time is suggestive (eg, confirmed by dechallenge). An alternative explanation is less likely, eg, concomitant drug(s), concomitant disease(s).

Very Likely: An adverse event that is listed as a possible adverse reaction and cannot be reasonably explained by an alternative explanation, eg, concomitant drug(s), concomitant disease(s). The relationship in time is very suggestive (eg, it is confirmed by dechallenge and rechallenge).

### Severity Criteria:

The severity assessment for an adverse event/serious adverse event was completed using the NCI-CTCAE Version 5.0. Any adverse event/serious adverse event not listed in the NCI-CTCAE was graded by the investigator using the standard grades as follows:

| GRADE | DEFINITION |
|---|---|
| 1 | Mild: Symptoms which do not interfere |
| | with subject's daily activities |
| 2 | Moderate: Symptoms which may interfere |
| | with subject's daily activities |
| 3 | Severe: Events which interrupt subject's |
| | usual daily activities |
| 4 | Life-threatening or disabling |
| 5 | Death |

The investigator was to use clinical judgment in assessing the severity of events not directly experienced by the subject (eg, laboratory abnormalities).

### Special Reporting Situations:

Safety events of interest on a sponsor study drug that require expedited reporting and/or safety evaluation included, but were not limited to:
Overdose of a sponsor medicinal product
Suspected abuse/misuse of a sponsor medicinal product
Accidental or occupational exposure to a sponsor medicinal product
Special reporting situations were recorded in the CRF. Any special reporting situation that met the criteria of a serious adverse event was recorded on the serious adverse event page of the CRF.

### Procedures:

### All Adverse Events:

All adverse events and special reporting situations, whether serious or non-serious, were reported from the time a signed and dated ICF was obtained until completion of the subject's last study-related procedure, which may include contact for follow up of safety. Serious adverse events, including those spontaneously reported to the investigator within 30 days after the last dose of study drug, were reported using the Serious Adverse Event Form. The sponsor was to evaluate any safety information that was spontaneously reported by an investigator beyond the time frame specified in the protocol.

All events that met the definition of a serious adverse event were reported as serious adverse events, regardless of whether they were protocol-specific assessments.

All adverse events, regardless of seriousness, severity, or presumed relationship to study therapy, were recorded using medical terminology in the source document and the CRF. Whenever possible, diagnoses were given when signs and symptoms were due to a common etiology (eg, cough, runny nose, sneezing, sore throat, and head congestion should be reported as “upper respiratory infection”). Investigators recorded in the CRF their opinion concerning the relationship of the adverse event to study therapy. All measures required for adverse event management were recorded in the source document and reported according to sponsor instructions.

The sponsor assumed responsibility for appropriate reporting of adverse events to the regulatory authorities. The sponsor also reported to the investigator (and the head of the investigational institute where required) all suspected unexpected serious adverse reactions (SUSARs). The investigator (or sponsor where required) reported SUSARs to the appropriate Independent Ethics Committee/Institutional Review Board (IEC/IRB) that approved the protocol unless otherwise required and documented by the IEC/IRB.

For all studies with an outpatient phase, including open-label studies, the subject was provided with a “wallet (study) card” and instructed to carry this card with them for the duration of the study indicating the following:
Study number
Statement, in the local language(s), that the subject is participating in a clinical study
Investigator's name and 24-hour contact telephone number
Local sponsor's name and 24-hour contact telephone number (for medical staff only)
Site number
Subject number

### Serious Adverse Events:

All serious adverse events occurring during clinical studies were reported to the appropriate sponsor contact person by investigational staff within 24 hours of their knowledge of the event.

Information regarding serious adverse events was transmitted to the sponsor using the Serious Adverse Event Form, which was completed and signed by a physician from the study center, and transmitted to the sponsor within 24 hours. The initial and follow up reports of a serious adverse event were made by facsimile (fax).

All serious adverse events that were not resolved by the end of the study, or that were not resolved upon discontinuation of the subject's participation in the study, were followed until any of the following occurs:
The event resolves
The event stabilizes
The event returns to baseline, if a baseline value/status is available
The event can be attributed to agents other than the study drug or to factors unrelated to study conduct
It becomes unlikely that any additional information can be obtained (subject or health care practitioner refusal to provide additional information, lost to follow up after demonstration of due diligence with follow up efforts) Suspected transmission of an infectious agent by a medicinal product will be reported as a serious adverse event. Any event requiring hospitalization (or prolongation of hospitalization) that occurs during the course of a subject's participation in a clinical study must be reported as a serious adverse event, except hospitalizations for the following:
Hospitalizations not intended to treat an acute illness or adverse event (eg, social reasons such as pending placement in long-term care facility)
Surgery or procedure planned before entry into the study (must be documented in the CRF). Note: Hospitalizations that were planned before the signing of the ICF, and where the underlying condition for which the hospitalization was planned has not worsened, will not be considered serious adverse events. Any adverse event that results in a prolongation of the originally planned hospitalization is to be reported as a new serious adverse event.
Protocol specified residence in the study center.

### Pregnancy:

The ongoing and planned clinical studies of apalutamide are in healthy men and men with advanced prostate cancer. Nevertheless, there could be risks to unborn children fathered by subjects receiving apalutamide.

There are no human data on the use of apalutamide in pregnancy. Based on its mechanism of action, maternal use of an AR antagonist is expected to affect the development of the fetus.

Because the effect of the study drug on sperm or fetus is unknown, all initial reports of pregnancy in partners of male subjects were reported to the sponsor by the study-site personnel within 24 hours of their knowledge of the event using the appropriate pregnancy notification form. Abnormal pregnancy outcomes (eg, spontaneous abortion, fetal death, stillbirth, congenital anomalies, ectopic pregnancy) were considered serious adverse events and must be reported using the Serious Adverse Event Form.

Follow up information regarding the outcome of the pregnancy and any postnatal sequelae in the infant were required.

### Contacting Sponsor Regarding Safety:

The names (and corresponding telephone numbers) of the individuals who should be contacted regarding safety issues or questions regarding the study were listed in the Contact Information page(s), which was provided as a separate document.

### Product Quality Complaint Handling:

A product quality complaint (PQC) was defined as any suspicion of a product defect related to manufacturing, labeling, or packaging, ie, any dissatisfaction relative to the identity, quality, durability, or reliability of a product, including its labeling or package integrity. PQCs may have an impact on the safety and efficacy of the product. Timely, accurate, and complete reporting and analysis of PQC information from clinical studies are crucial for the protection of subjects, investigators, and the sponsor, and are mandated by regulatory agencies worldwide. The sponsor has established procedures in conformity with regulatory requirements worldwide to ensure appropriate reporting of PQC information; all clinical studies conducted by the sponsor or its affiliates were conducted in accordance with those procedures.

### Procedures:

All initial PQCs were reported to the sponsor by the investigational staff within 24 hours after being made aware of the event.

If the defect was combined with a serious adverse event, the investigational staff reported the PQC to the sponsor according to the serious adverse event reporting timelines (refer to Section Error! Reference source not found., Serious Adverse Events). A sample of the suspected product was maintained for further investigation if requested by the sponsor.

### Contacting Sponsor Regarding Product Quality:

The names (and corresponding telephone numbers) of the individuals who should be contacted regarding product quality issues were listed in the Contact Information page(s), which was provided as a separate document.

### Study Specific Materials

The investigator was provided with the following supplies:
Package Insert for apalutamide
Pharmacy manual/study center investigational product and procedures manual
Laboratory manual
NCI-CTCAE Version 5.0
Sample ICF
PK Labels
eSource Manual

### Ethical Aspects

### Study-Specific Design Considerations:

Potential subjects were fully informed of the risks and requirements of the study and, during the study, subjects were given any new information that may affect their decision to continue participation. They were told that their consent to participate in the study is voluntary and may be withdrawn at any time with no reason given and without penalty or loss of benefits to which they would otherwise be entitled. Only subjects who were fully able to understand the risks, benefits, and potential adverse events of the study, and provide their consent voluntarily were enrolled.

This study was being conducted to evaluate the bioavailability of apalutamide tablets administered orally as dispersed tablets mixed in applesauce in comparison to whole tablets under fasting conditions.

The primary ethical concern was that this study be performed in healthy male subjects who will receive no benefit from participation in the study, except for financial compensation for the time and inconveniences that may arise from participation in the study.

### Safety of Dose:

The potential risks to subjects in this study included exposure to study drugs, with a potential for AEs. In previous clinical studies in healthy subjects, administration of a single dose of apalutamide up to 240 mg was well tolerated. Subjects were monitored for AEs throughout the study, and an expert investigator was chosen to conduct the study. In each treatment period, subjects were confined to the study center until completion of the 72-hour PK assessments on Day 4 and returned to the study center for additional assessments up to Day 8 after intake of the study drug. Subject's safety was monitored at regular intervals throughout the study by physical examination, laboratory safety assessments, ECG and vital signs.

### Pharmacokinetic Blood Sampling:

As with all clinical PK studies, there are risks associated with venipuncture and multiple blood sample collection. The blood sample collection scheme was designed to collect the number of blood samples required to accurately and completely describe the PK of the study drugs and evaluate the bioavailability of apalutamide tablets dispersed in applesauce, compared to whole tablets. This minimizes the total volume of blood (approximately 200 mL) collected from each subject. All of the blood samples were collected over an 84-day period.

The total blood volume to be collected was considered to be an acceptable amount of blood to be collected over this time period from the population.

### Study Completion/Termination

### Study Completion/End of Study:

The study was considered completed with the last scheduled study assessment shown in the Time & Event Schedule for the last subject participating in the study. The final data from the investigational site was sent to the sponsor (or designee) after completion of the final subject assessment at that site, in the time frame specified in the Clinical Trial Agreement.

### Study Termination:

The sponsor reserved the right to close the study-site or terminate the study at any time for any reason at the sole discretion of the sponsor. Study centers were closed upon study completion. A study center was considered closed when all required documents and study supplies had been collected and a study-site closure visit was performed.

The investigator could initiate site closure at any time, provided there was reasonable cause and sufficient notice was given in advance of the intended termination.

Reasons for the early closure of an study-site by the sponsor or investigator could include, but were not limited to:
Failure of the investigator to comply with the protocol, the requirements of the IEC/IRB or local health authorities, the sponsor's procedures, or GCP guidelines.
Inadequate recruitment of subjects by the investigator.
Discontinuation of further drug development.

### On-Site Audits:

Representatives of the sponsor's clinical quality assurance department could visit the study center at any time during or after completion of the study to conduct an audit of the study in compliance with regulatory guidelines and company policy. These audits would require access to all study records, including source documents, for inspection. Subject privacy must, however, be respected. The investigator and study-site personnel were responsible for being present and available for consultation during routinely scheduled study-site audit visits conducted by the sponsor or its designees.

Similar auditing procedures could also be conducted by agents of any regulatory body, either as part of a national GCP compliance program or to review the results of this study in support of a regulatory submission. The investigator was to immediately notify the sponsor if he or she has been contacted by a regulatory agency concerning an upcoming inspection.

### Use of Information and Publication:

All information, including but not limited to information regarding apalutamide or the sponsor's operations (eg, patent application, formulas, manufacturing processes, basic scientific data, prior clinical data, formulation information) supplied by the sponsor to the investigator and not previously published, and any data generated as a result of this study, are considered confidential and remain the sole property of the sponsor. The investigator agreed to maintain this information in confidence and use this information only to accomplish this study, and would not use it for other purposes without the sponsor's prior written consent.

The investigator understands that the information developed in the study will be used by the sponsor in connection with the continued development of apalutamide, and thus may be disclosed as required to other clinical investigators or regulatory agencies. To permit the information derived from the clinical studies to be used, the investigator was obligated to provide the sponsor with all data obtained in the study.

The results of the study will be reported in a Clinical Study Report generated by the sponsor and will contain data from all study centers that participated in the study a per protocol. Recruitment performance or specific expertise related to the nature and the key assessment parameters of the study were used to determine a coordinating investigator. Study subject identifiers were not used in publication of results. Any work created in connection with performance of the study and contained in the data that can benefit from copyright protection (except any publication by the investigator as provided for below) shall be the property of the sponsor as author and owner of copyright in such work.

Consistent with Good Publication Practices and International Committee of Medical Journal Editors guidelines, the sponsor shall have the right to publish such primary (single- and multicenter) data and information without approval from the investigator. The investigator has the right to publish study center-specific data after the primary data are published. If an investigator wishes to publish information from the study, a copy of the manuscript must be provided to the sponsor for review at least 60 days before submission for publication or presentation. Expedited reviews will be arranged for abstracts, poster presentations, or other materials. If requested by the sponsor in writing, the investigator will withhold such publication for up to an additional 60 days to allow for filing of a patent application. In the event that issues arise regarding scientific integrity or regulatory compliance, the sponsor will review these issues with the investigator. The sponsor will not mandate modifications to scientific content and does not have the right to suppress information. For multicenter study designs and substudy approaches, secondary results generally should not be published before the primary endpoints of a study have been published. Similarly, investigators will recognize the integrity of a multicenter study by not submitting for publication data derived from the individual study center until the combined results from the completed study have been submitted for publication, within 12 months of the availability of the final data (tables, listings, graphs), or the sponsor confirms there will be no multicenter study publication. Authorship of publications resulting from this study will be based on the guidelines on authorship, such as those described in the Uniform Requirements for Manuscripts Submitted to Biomedical Journals, which state that the named authors must have made a significant contribution to the design of the study or analysis and interpretation of the data, provided critical review of the paper, and given final approval of the final version.

### Results

The study described above was conducted from 14 Jan. 2019 to 29 Apr. 2019. No major protocol deviations were reported.

### Patient Population

In total, 12 subjects were randomized to receive Treatment A (240 mg apalutamide, swallowed whole fasted) and Treatment B (240 mg apalutamide, tablets dispersed mixture in applesauce, fasted) in a crossover (2 periods) manner separated by a washout interval of at least 43 days and no more than 46 days between doses, of which 10 (83.3%) subjects completed the study. Two subjects discontinued from the study for the following reasons: 1 (8.3%) subject withdrew due to personal reasons after Treatment A in Period 1, and 1 (8.3%) subject discontinued the study due to the AE gynecomastia after Treatment B in Period 1. Subject disposition was as listed in Table R1 and shown in FIG. 3.

TABLE R1
Subject Disposition

| | Randomization | | Treatment | | | | |
|---|---|---|---|---|---|---|---|
| | Sequence | | Period 1 | | Period 2 | | |
| | A-B | B-A | A | B | A | B | Total |
| Analysis set: | 6 | 6 | 6 | 6 | 5 | 5 | 12 |
| Safety | | | | | | | |
| Subjects | 6 (100%) | 6 (100%) | 6 (100%) | 6 (100%) | 5 (100%) | 5 (100%) | 12 (100%) |
| randomized | | | | | | | |
| Completed study | 5 (83.3%) | 5 (83.3%) | 5 (83.3%) | 5 (83.3%) | 5 (100%) | 5 (100%) | 10 (83.3%) |
| participation | | | | | | | |
| Terminated | 1 (16.7%) | 1 (16.7%) | 1 (16.7%) | 1 (16.7%) | 0 | 0 | 2 (16.7%) |
| study | | | | | | | |
| participation | | | | | | | |
| prematurely | | | | | | | |
| Reason for | | | | | | | |
| termination | | | | | | | |
| Adverse event | 0 | 1 (16.7%) | 0 | 1 (16.7%) | 0 | 0 | 1 (8.3%) |
| Withdrawal by | 1 (16.7%) | 0 | 1 (16.7%) | 0 | 0 | 0 | 1 (8.3%) |
| subject | | | | | | | |

Treatment A = 240 mg apalutamide commercial formulation single dose of 4 × 60 mg tablets swallowed whole, fasted.
Treatment B = 240 mg apalutamide commercial formulation single dose of 4 × 60 mg tablets dispersed mixture in applesauce, fasted.

The demographic and baseline characteristics of subjects were as shown in FIG. 4. Overall, 12 white male subjects were enrolled in this study. The median age was 49 years (range: 21 to 55 years). The median BMI was 24.2 kg/m2 (range: 22.6 to 28.6 kg/m2). Demographic and baseline characteristics were consistent with the inclusion and exclusion criteria described in the protocol. No clinically significant medical history or physical examination abnormalities that would have precluded enrollment and participation were reported in this study.

No pre-study medications were reported for any subjects m this study. One (8.3%) subject received the following concomitant medication: etofenamate (one application) and paracetamol (500 mg, once) for the treatment of the TEAE bursitis during Treatment A in Period 1.

Study drugs were administered by site personnel to assure compliance with study requirements. All subjects received 240 mg apalutamide on 2 occasions, except for 2 subjects (1 from each treatment sequence). One subject discontinued the study after receiving Treatment A (240 mg apalutamide, swallowed whole fasted) due to personal reasons. The second subject prematurely discontinued the study due to an AE (gynecomastia) after receiving Treatment B (240 mg apalutamide, tablets dispersed mixture in applesauce, fasted).

### Pharmacokinetic Results

The plasma concentrations of apalutamide were determined using a validated, specific, and sensitive LC-MS/MS method with a LLOQ of 0.0250˜g/mL. Two subjects only completed Period 1 of the study and consequently, no data were available in Period 2. One of the subjects withdrew for personal reasons and the other one withdrew due to an AE. All other scheduled samples were available for bioanalysis.

A total of 12 subjects were included in the Pharmacokinetics Data Analysis Set. The actual randomization codes were as shown in the Table of FIG. 5. Only data from subjects who completed both treatments were considered in the PK inferential statistical analysis (comparative statistics). Two subjects did not complete the study. therefore, only 10 subjects were included in the Pharmacokinetics Data Statistical Analysis Set.

Apalutamide pre-dose plasma concentration was quantifiable for two subjects in Period 2, which may be attributed to a slow elimination of the drug administered in Period 1 and an insufficient washout period between both treatment periods. However, the data were not excluded from analysis since the quantifiable pre-dose concentrations were <5% of Cmax. No vomiting episodes were reported. For one subject, a diarrhea episode started almost 5 days after Treatment B administration. Data were kept in analyses since no impact was expected.

The PK parameters were calculated using the actual blood sampling times. All PK samples were collected within the allowed time window: there were no actual sampling times deviating by >20% from the scheduled (nominal) time.

### Plasma Concentration-Time Profiles

Individual plasma apalutamide concentrations with descriptive statistics are provided in FIG. 6 and FIG. 7. Mean plasma concentration-time profiles were as shown in FIG. 8.

Apalutamide was quantifiable in the first collected post-dose sample (0.5 hour post-dose), except for the samples of two subjects in Treatment A (sequence A-B) which were only quantifiable beginning at I hour post-dose. Apalutamide plasma concentrations reached a maximum 3 hours after administration of Treatment A and 2 hours after administration of Treatment B. On average, apalutamide plasma levels were higher in Treatment B compared to Treatment A until about 4 hours post-dose. Thereafter, the plasma concentrations were comparable in both treatments. Apalutamide plasma concentrations decreased in a multi-phasic manner and were still quantifiable at the last PK sample (168 hours post-dose) for both treatments and for all subjects.

### PK Parameters

Individual apalutamide PK parameters with descriptive statistics are provided in FIG. 9 (Treatment A) and FIG. 10 (Treatment B), and summarized in Table R2, below.

TABLE R2
Pharmacokinetic Results of Apalutamide After Single
Oral Administration of 240 mg Apalutamide Tablets
Swallowed Whole Under Fasted Conditions (Treatment A)
or as a Dispersed Mixture in Applesauce (Treatment B)

| Pharmacokinetics of | Swallowed Whole | | Dispersed in | |
|---|---|---|---|---|
| Apalutamide | Fasted | | Applesauce | |
| mean (SD), tₘₐₓ: | (Treatment A) | | (Treatment B) | |
| median (range) | (Reference) | | (Test) | |
| n | 11 | | 11 | |
| Cₘₐₓ (μg/mL) | 1.91 | (0.521) | 2.35 | (0.560) |
| tₘₐₓ (h) | 3.00 | (2.00-8.00) | 2.00 | (1.00-4.00) |
| AUC_{0-72 h} (μg · h/mL) | 61.3 | (12.8) | 62.5 | (13.0) |
| AUC_{0-168 h} (μg · h/mL) | 110 | (20.9) | 112 | (21.1) |

Individual PK parameter treatment ratios with descriptive statistics were as listed in FIG. 11. The median tmax of apalutamide after administration of Treatment B was 1 hour less than Treatment A (2 vs 3 hours).

The results of statistical analysis (assessment of relative bioavailability of Treatment B with respect to the reference Treatment A) for the In-transformed apalutamide PK parameters (Cmax: AUC0-72 h and AUC0-168 h) are provided in Table R3, below.

TABLE R3
Summary of the Statistical Analysis of the Pharmacokinetic Parameters of
Apalutamide After Single Oral Administration of 240 mg Apalutamide Tablets
Swallowed Whole Under Fasted Conditions or as a Dispersed Mixture in Applesauce

| | | Geometric Means | | | | | |
|---|---|---|---|---|---|---|---|
| | | Swallowed | Dispersed | | | | |
| | | Whole | in | Dispersed in applesauce versus | | | |
| | | Fasted | Applesauce | swallowed whole fasted (B/A) | | | |
| | | (Treatment | (Treatment | Geometric | Lower | Upper | Intra- |
| | | A) | B) | Mean | Limit 90% | Limit 90% | subject |
| PK Parameter | n | (Reference) | (Test) | Ratio (%) | CI (%) | CI (%) | CV(%) |
| Cₘₐₓ (μg/mL) | 10 | 1.80 | 2.30 | 127.57 | 113.76 | 143.05 | 13.8 |
| AUC₀₋₇₂ₕ | 10 | 58.6 | 61.5 | 105.02 | 101.87 | 108.27 | 3.7 |
| (μg · h/mL) | | | | | | | |
| AUC₀₋₁₆₈ₕ | 10 | 106 | 111 | 105.22 | 102.88 | 107.60 | 2.7 |
| (μg · h/mL) | | | | | | | |

The administration of apalutamide either as a dispersed mixture in applesauce (Treatment B) or as standard oral administration (Treatment A) showed comparable exposures as shown by the 90% CI of the geometric mean ratio of AUC0-168 h values contained within the 80%-120% limit. Cmax was increased by 27.6% when apalutamide was administered as a dispersed mixture in applesauce compared to standard oral administration of tablets. The intra-subject variability in the apalutamide PK parameters was low for Cmax. AUC0-72 h and AUC0-168 h, with intra-subject CV ranging from 2.7% to 13.8%.

### Safety Results

The safety analysis set included all subjects who received at least 1 dose of study agent.

### Summary of All Adverse Events

The overall safety profile, based on TEAEs, was as presented in Table R4, below.

TABLE R4

| | Treatment A | Treatment B | Total |
|---|---|---|---|
| Number of Subjects Treated | 11 | 11 | 12 |
| Treatment Emergent Adverse Events | 3 (27.3%) | 5 (45.5%) | 5 (41.7%) |
| Drug-related to Treatment A^{a} | 3 (27.3%) | 1 (9.1%) | 4 (33.3%) |
| Drug-related to Treatment B^{a} | 1 (9.1%) | 4 (36.4%) | 4 (33.3%) |
| Grade 1^{b} | 2 (18.2%) | 5 (45.5%) | 4 (33.3%) |
| Grade 2^{b} | 1 (9.1%) | 0 | 1 (8.3%) |
| >=Grade 3^{b} | 0 | 0 | 0 |
| Serious Treatment Emergent | 0 | 0 | 0 |
| Adverse Events | | | |
| Drug-related to Treatment A^{a} | 0 | 0 | 0 |
| Drug-related to Treatment B^{a} | 0 | 0 | 0 |
| Grade 1^{b} | 0 | 0 | 0 |
| Grade 2^{b} | 0 | 0 | 0 |
| >=Grade 3^{b} | 0 | 0 | 0 |
| AE leading to treatment | 0 | 1 (9.1%) | 1 (8.3%) |
| discontinuation | | | |
| Drug-related to Treatment A^{a} | 0 | 0 | 0 |
| Drug-related to Treatment B^{a} | 0 | 1 (9.1%) | 1 (8.3%) |
| Grade 1^{b} | 0 | 1 (9.1%) | 1 (8.3%) |
| Grade 2^{b} | 0 | 0 | 0 |
| >=Grade 3^{b} | 0 | 0 | 0 |

Key:
AE = adverse event
Note:
^{a}Drug related adverse events are AEs with causality reported as possible, probable or very likely to either Treatment A or B.
Note:
^{b}Only subjects with this severity grade as worst grade are counted.
A = 240 mg apalutamide commercial formulation single dose of 4 × 60 mg tablets swallowed whole, fasted.
B = 240 mg apalutamide commercial formulation single dose of 4 × 60 mg tablets dispersed mixture in applesauce, fasted.

Across both treatment sequences, 5 (41.7%) subjects reported at least 1 TEAE, including 3 (27.3%) subjects after Treatment A and 5 (45.5%) subjects after Treatment B (Table 4). The majority of TEAEs were Grade 1 (33.3% of subjects). There was one (8.3%) subject with a Grade 2 AE and no subjects with Grade 3 or 4 AEs. The summary of TEAEs by system organ class and preferred term, by treatment group, are shown in FIG. 12, and total (for both treatment groups) in FIG. 13; safety analysis set. The most common TEAE was gynecomastia reported in 3 (25.0%) subjects; all other TEAEs were reported in at most 1 (8.3%) subject.

Following Treatment A (240 mg apalutamide, swallowed whole fasted):
No AEs were reported in more than 1 (9.1%) subject.
One subject (9.1%) reported a Grade 2 AE (bursitis), which was considered not drug-related by the investigator.
Three (27.3%) subjects reported a Grade 1 AE that was considered drug-related to Treatment A by the investigator including hot flush, rash vesicular and gynecomastia (one subject each).

Following Treatment B (240 mg apalutamide, tablets dispersed mixture in applesauce, fasted):
No AEs were reported in more than 1 (9.1%) subject, except for the AE gynecomastia which was reported in 2 (18.2%) subjects.
All AEs were Grade 1 in severity.
Four (36.4%) subjects were reported with a Grade 1 AE that was considered drug-related to treatment B by the investigator including gynecomastia (2 subjects}, rash generalized and rash vesicular (1 subject each).

Sexual side effects were reported in 3 (25.0%) subjects: Grade 1 gynecomastia was reported in all three subjects. Two subjects spontaneously reported the TEAE gynecomastia after study completion (one subject reported the TEAE 17 days following Treatment B [Period 2] and one subject 45 days following Treatment A [Period 2]). Both TEAEs were considered related to both Treatment A and B by the investigator and ongoing at time of database lock. One subject was reported with gynecomastia after 29 days following the first dose of apalutamide (Treatment B). The TEAE was considered drug-related and ongoing at time of database lock. Further follow-up demonstrated that all gynecomastia TEAEs were resolved 90 to 125 days after start of the event. No other sexual side effects were reported.

There were no deaths or serious TEAEs reported in this study.

A 21-year-old White male subject, who was randomized to treatment sequence B-A had a TEAE (Grade 1 gynecomastia) leading to study drug discontinuation. The subject was reported with gynecomastia (preferred term: gynecomastia vera, bilateral) 29 days after study drug intake. The AE was mild in severity, considered drug-related by the investigator, and resolved after 112 days. The subject did not receive Treatment A.

### Clinical Laboratory Evaluation

Laboratory values (including albumin, bicarbonate, chloride, creatine kinase, creatinine, phosphate, potassium, protein, sodium, urate, cholesterol, glucose, triglycerides, alanine aminotransferase, alkaline phosphatase, aspartate aminotransferase, bilirubin, direct bilirubin, gamma glutamyl transferase, indirect bilirubin, lactate dehydrogenase, basophils, basophils/leukocytes, eosinophils, eposinophils/leukocytes, erythrocytes, hematocrit, hemoglobin, leukocytes, lymphocytes, lymphocytes/leukocytes, monocytes, monocytes/leukocytes, neutrophils and platelets) and changes from baseline (last observation prior to first study drug administration) over time were measured. None of the median changes from baseline in laboratory parameters were considered to be clinically relevant.

### Hematology & Biochemistry

Few subjects showed occasional shifts in hematology laboratory values during the study, but these were considered not clinically significant by the investigator:
All subjects entered the study with normal or Grade 1 test results for hematology and biochemistry parameters at baseline except for 1 subject with a Grade 2 decreased neutrophil count at screening.
Three (16.7%) subjects had a hematology related abnormality of Grade 2 or higher during the study. One subject had a Grade 2 leukocyte count decreased during day 2 of Treatment B in Period 2. This subject had a Grade 1 leukocyte count decreased at baseline and no abnormal leukocyte count was observed on Day 8 of Period 2. One subject had a Grade 2 lymphocyte count decreased on Day −1 of Treatment B in Period 2. This subject had a Grade 1 lymphocyte count decreased at baseline and no abnormal lymphocyte count was observed on Day 8 of Period 2. The other Grade 2 abnormality was a decreased neutrophil count on Day 8 of Period 1 and Day-1 of Period 2 (treatment sequence A-B). The subject was reported with a Grade 2 decreased neutrophil count at screening.
One subject had a biochemistry related abnormality of Grade 2 during the study. The subject experienced a Grade 2 elevated creatine kinase on Day 8 of Treatment A in Period 2. The increase in creatine kinase was related to excessive physical activity and considered not drug-related by the investigator.
Treatment-emergent non-graded hematology and biochemistry values were reported in at most 2 subjects per treatment group except for an abnormally high bicarbonate reported in 5 (45.5%) subjects after Treatment A and 5 (45.5%) subjects after Treatment B.

### Liver Function Tests

Few subjects showed occasional shifts in liver function values during the study, but these were considered not clinically significant:
No subject had a liver function test abnormality of Grade 2 or higher during the study, except for one (8.3%) subject with a Grade 2 (34.2˜μl/L) increased bilirubin on day 2 of Period 1 (Treatment A). This subject had a Grade 1 (25.65 μmol/L) increased bilirubin at baseline and no abnormal bilirubin value (15.39 μmol/L) was observed on Day 8 of Period 2 (Treatment B).
There was one (8.3%) subject with a Grade 1 (53 U/L) alanine aminotransferase increased on the day 8 visit after Treatment A in Period 1. No other relevant liver function test abnormalities were reported at any other timepoint for this subject.
Treatment-emergent non-graded values were reported in at most 2 subjects per treatment group except for an abnormally low lactate dehydrogenase reported in 1 (9.1%) subject after Treatment A and 3 (27.3%) subjects after Treatment B.

### Lipid and Glucose Tests

Few subjects showed occasional shifts in lipid and glucose test values during the study but these were considered not clinically significant:
No subject had a lipid or glucose test abnormality of Grade 2 or higher during the study.
Two subjects were reported with an abnormal lipid test (Grade 1 elevated cholesterol) on the day 8 visit after both Treatment A and B. Both these subjects also had Grade 1 elevated cholesterol tests at screening. No subjects were reported with a glucose abnormality on the day 8 visit after intake of either Treatment A or B.

### Individual Clinically Significant Abnormalities

None of the observed changes in liver function test, lipids, glucose tests, biochemistry and hematology were considered clinically significant by the investigator and reported as TEAE.

### Vital Signs and Physical Examination Findings

Mean changes over time in vital signs parameters (including blood pressure, systolic blood pressure, diastolic blood pressure, body temperature, respiratory rate) were generally minor and not considered to be clinically relevant.

The observed treatment-emergent vital signs abnormalities in Treatment A were a low systolic blood pressure (<90 mmHg) in 1 (9.1%) subject (Period 1, day 8), a low diastolic blood pressure (<60 mm·Hg) in 1 (9.1%) subject (Period 2, day −1), a low body temperature (<36° C.) in 2 (18.2%) subjects (Period 1, day 2 and Period 2 day −1) and a low respiratory rate (<12 breaths/min) in 1 (9.1%) subject (Period 1 day 8). After Treatment B a low systolic blood pressure in 1 (9.1%) subject (Period 2 day 8), a low diastolic blood pressure in 1 (9.1%) subject (Period 2, day 8), a low body temperature 2 (18.2%) subjects (Period 1 day 2 and Period 2 days 2 and 8) and a low respiratory rate in 4 (36.4%) subjects (Period 1 day 8 and Period 2 day −1), were observed.

None of the reported vital signs abnormalities were considered clinically significant. No TEAEs related to vital signs parameters were reported in the study.

All clinically significant treatment-emergent physical examination findings were reported by the investigator as AE.

### Electrocardiograms

Mean values and mean change from baseline for key cardiovascular parameters included measurement of heart rate, PR, QRS, QTc (Bazett), QTc (Fridericia) and uncorrected QT.

No abnormality was observed in any of the ECG values (heart rate, PR, QRS, QTcB and QTcF). After Treatment B, 2 (18.2%) subjects had a QTcF increase of 30-60 ms on Day 8˜one subject during Period 1 and one subject during Period 2. No subjects had QTcF increase of >30 ms after Treatment A. No subjects with QTcB or QTcF values of >480 ms were observed in the study. No subjects had QTcB or QTcF increase of >60 ms from baseline in the study.

No clinically meaningful treatment-related trends in ECG parameters were observed in this study.

### Palatability

A summary of the taste questionnaire results is shown in FIG. 14. The overall acceptability of Treatment B ranged from “maybe bad maybe good” in 1 (9.1%) subject to “super good” in 3 (27.3%) subjects. Most of the subjects (90.9%) did not find it annoying to swallow the substance and the taste of the applesauce mixture was “sweet” or “pleasant”. The bitterness and the smell ranged from “good” to “super good”. All subjects reported that it was acceptable for long period use.

### Results Discussion

This was a randomized, open-label, balanced, single dose, two-treatment, two-period, two-sequence, crossover relative bioavailability study. Healthy male subjects were administered a single dose of apalutamide 240 mg on 2 separate occasions either as whole tablets or as dispersed mixture in applesauce. In total, 12 subjects were randomized to receive Treatment A and Treatment B in a crossover (2 periods) manner Two subjects discontinued from the study for the following reasons: 1 (8.3%) subject withdrew due to personal reasons not related to the study or an AE after Treatment A (treatment sequence A-B) and 1 (8.3%) subject discontinued the study due to a Grade 1 TEAE gynecomastia after Treatment B (treatment sequence B-A).

### Pharmacokinetics

The extents of absorption as assessed by AUC0-168 h after administration of apalutamide as a dispersed mixture in applesauce (Treatment B) and as a standard oral administration of tablets (Treatment A) are highly comparable with the 90% CI meeting the 80%-125% criterion for bioequivalence. The mean AUC0-168 h for Treatment A (110 μg·h/mL) and Treatment B (112 μg·h/mL) are comparable to AUC0-168 h (121 μg·h/mL) from another bioavailability study at a same dose of 240 mg (N=15) of apalutamide. While the difference between Treatment B and Treatment A has been found to be statistically significant at 5%, the difference is not expected to be clinically significant.

The observed peak apalutamide concentration (Cmax) after Treatment B is 27.6% higher than the Cmax after Treatment A. The median time to reach Cmax (tmax) after Treatment B (2 hours) is shorter compared to the median tmax after Treatment A. These higher Cmax and shorter tmax for Treatment B can possibly be attributed to the shift from an in vivo disintegration of the tablets for Treatment A to an ex vivo disintegration of the tablets in applesauce for Treatment B. The ex vivo disintegration of tablets in applesauce could have provided already disintegrated and dissolved drug particles for oral intake, resulting in earlier and faster systemic absorption of apalutamide.

When compared to the mean Cmax of 2.42 μg/mL after administration of 240 mg apalutamide from a similar study, the Cmax for Treatment B was comparable at 2.35 μg/mL but the Cmax for Treatment A was lower at 1.91 μg/mL. This supports the fact that although in this study the difference in Cmax between Treatment B and Treatment A are statistically significant (median of 127.57% with 90% CI: 113.76-143.05%), overall the Cmax values are within the range of inter-subject and/or inter-study variability.

### Safety

Across both treatment sequences, 5 (41.7%) subjects reported at least 1 TEAE, including 3 (27.3%) subjects after Treatment A (240 mg apalutamide, swallowed whole fasted) and 5 (45.5%) subjects after Treatment B (240 mg apalutamide, tablets dispersed mixture in applesauce, fasted). All TEAEs were of Grade 1 (33.3% of subjects) in severity except for 1 (8.3%) subject who reported a Grade 2 TEAE of bursitis after treatment A, which was considered not drug-related by the investigator. Drug-related TEAEs were reported by 3 (27.3%) subjects after Treatment A and 4 (36.4%) subjects after Treatment B. The most common TEAE was gynecomastia reported in 3 (25.0%) subjects; all other TEAEs were reported in at most 1 subject. Two subjects spontaneously reported the TEAE gynecomastia after study completion (one subject reported the TEAE 17 days following Treatment B [Period 2] and one subject 45 days following Treatment A [Period 2]). Both TEAEs were of severity Grade 1, considered drug-related to Treatment A and B by the investigator and ongoing at time of database lock. One subject was reported with gynecomastia after 29 days following the first dose of apalutamide (Treatment B). The TEAE was of Grade 1 in severity, considered drug-related and ongoing at time of database lock. Further follow-up demonstrated that all gynecomastia TEAEs were resolved 90 to 125 days after the start of the event. No other sexual side effects were reported.

Overall, there were no clinically meaningful, treatment-related mean changes from reference in vital signs, ECG parameters, and safety laboratory parameters.

### Palatability

The overall acceptability of Treatment B ranged from “maybe bad maybe good” in 1 (9.1%) subject to “super good” in 3 (27.3%) subjects. Most of the subjects (90.9%) did not find it annoying to swallow the substance and the taste of the medicine was “sweet” or “pleasant”.

The bitterness and the smell ranged from “good” to “super good”. All subjects reported that it was acceptable for long period use.

### CONCLUSIONS

The intake of 240 mg of apalutamide administered as whole tablets and as dispersed tablets mixed in applesauce was safe and generally well tolerated. A higher incidence of TEAE gynecomastia has been observed (25% of the subjects) in this study, which has not been reported in prior clinical studies with single dose administration of 240 mg apalutamide in healthy volunteers. Otherwise the AE profile was as expected and no new safety signals were noted.

Administration of apalutamide either as a dispersed mixture in applesauce (Treatment B) or as standard oral administration (Treatment A) showed comparable exposures as shown by the 90% CI of the geometric mean ratio for AUC0-168 h values contained within the 80%-125% limit. Administration as a dispersed mixture of apalutamide in applesauce resulted in higher Cmax (27.6%) and shorter tmax (by 1 hour) compared to standard oral administration of tablets. Both AUC0-168 h and Cmax values were within the similar range of previously reported results from earlier studies.

Administration of apalutamide using applesauce as a food vehicle is expected to be similar to standard oral administration for patients taking apalutamide daily.

### Example 2—Prophetic Example

Enzalutamide is evaluated as described in the protocol of Example 1, substituting one or more suitably dosage amount(s) of enzalutamide for the 240 mg of apalutamide.

### Example 3—Prophetic Example

Darolutamide is evaluated as described in the protocol of Example 1, substituting one or more suitably dosage amount(s) of enzalutamide for the 240 mg of apalutamide.

### Examples 4-7: Apalutamide Dispersions Dosing Accuracy, Chemical Stability and Visual Appearance

The chemical stability and dosing accuracy of a dispersed tablet mixture of four 60 mg film coated tablets of apalutamide, dispersed in selected suitable vehicles (including applesauce, green tea, orange juice and yogurt) at between about 50 mL and about 200 mL (or at between about 50 g to about 200 g) were evaluated as described in more detail below.

The 60-mg apalutamide tablets were oral coated tablets containing 60 mg of apalutamide as a SDP (solid dispersion) in hydroxypropyl methylcellulose-acetate succinate (HPMC-AS) polymer, in a 1/3 ratio (API/polymer). This oral coated tablet also contained the following inactive ingredients: colloidal anhydrous silica, croscarmellose sodium, microcrystalline cellulose, silicified microcrystalline cellulose, magnesium stearate and coating powder green OPADRY II. The tablet core weight was 700 mg.

The following test parameters were evaluated: (a) Appearance of Dispersion; (b) Assay of apalutamide at T0, 3 hours and 6 hours; and (c) Chromatographic Purity. Appearance was determined visually. Dosing accuracy and chemical stability (the concentration and impurities) were measured using the following UHPLC Procedure.

The reagents used in the UHPLC procedure were demineralized water, ammonium acetate, trifluoroacetic acid and acetonitrile. Mobile Phase A was a 10 mM NH₄Ac+01% TFA/Acetonitrile (90/10 v/v) solution prepared as follows. 0.77 g Ammonium acetate (NH₄Ac) was transferred into a 1000 mL volumetric flask. Milli-Q water was added to dissolve and dilute to a final volume of 1000 mL. 1.0 mL Trifluoroacetic acid (TFA) was then added via volumetric pipette and the resulting solution was mixed, then filtered through a 0.2 um Nylon filter. 900 mL of the filtrate was mixed with 100 mL acetonitrile to yield 1000 mL of Mobile Phase A. Mobile Phase B was acetonitrile. Sample solutions were prepared as described in the individual examples which follow hereinafter.

UHPLC conditions for dosing accuracy (assay) and chromatographic purity were as listed in Table P-1, below.

TABLE P-1
UHPLC Conditions

| | |
|---|---|
| Column | Acquity BEH C18, |
| | 150 mm length × 2.1 mm i.d., |
| | 1.7 μm particle size, P/N 186002353 |
| Column Temperature | 55° C. |
| Auto-Sampler Temperature | 20° C. |
| Flow Rate | 0.45 mL/min |
| Detection | UV |
| Wavelength | 268 nm |
| Injection Volume | 3 μL |
| Data Collection Time | 35 min |
| Analysis Run time | 40 min |
| Elution Mode | Gradient |

Linear Gradient:

| Time (min) | Mobile Phase A:B (% vol:% vol) |
|---|---|
| 0 | 100:0 |
| 35 | 30:70 |
| 36 | 100:0 |
| 50 | 100:0 |

For chemical stability (chromatographic characteristics) the identity of impurities were assigned based on comparison of their Relative Retention Times (RRT) to known retention times, or retention times determined using reference solutions. For the UHPLC assay measurements, a solution of 120 mg apalutamide in 250 mL acetonitrile (Reference Solution A) was used as a positive control/reference.

The representative, suitable vehicles tested were as follows:
Applesauce: Common US Brand: Motts
Applesauce: Common BE brand: Queen's Jewel (Aldi)
Green Tea Extract^{a}: Common Globally available brand of Green tea: Tetley Green tea refreshing pure original
Orange Juice: Common Globally available brand of orange juice: Minute Maid Orange juice
Yogurt: Common Indian brand Greek Yogurt: Epigamia
Yogurt: Common Indian brand Yogurt: Gowardhan

The green tea (as an extract of green tea leaves) was prepared as follow. 10 Green tea bags were dipped into 1000 mL of hot water at about 85° C. After about 2 min (steeping) the tea bags were taken out. The content remaining in the beaker was the final extract which was used for study. This preparation was consisting with the information on the label, which indicated that one tea bag is to be extracted per about 100 mL of hot water.

### Example 4: Applesauce

### Preparation of Applesauce Dispersions

The following steps were followed to disperse tablets of apalutamide in applesauce. Applesauce (50 g or 200 g) was added to a tared HDPE container. Four 60 mg tablets of apalutamide were removed from their packaging and placed in the applesauce. The mixture was gently mixed in the container with a spatula for 10 seconds and the time recorded. The hold time of the sample was calculated against the moment the tablets were added to the mixture. After 15 minutes, the mixture was again gently mixed with a spatula for 10 seconds and the time recorded. After an additional 15 minutes, the mixture was again gently mixed with a spatula for until a uniform mixture was formed (at least 10 seconds) and the time recorded.

### Dose Accuracy

Assay solution samples for dose accuracy were prepared as follows. The container containing the dispersion was emptied into a volumetric flask using a spoon. To the empty container was then added 60 mL of water, which was swirled gently to rinse the container. The first rinse was added to the volumetric flask. An additional 60 mL of water was added to the container and gently swirled to rinse; and the second rinse also added to the volumetric flask.

The dose accuracy was measured in triplicate for each applesauce dispersion mixture (50 g and 200 g) according to UHPLC Procedure described above. A total of 12 results were generated, which are reported relative to the determined assay of an apalutamide reference sample (i.e. apalutamide not mixed with applesauce or other suitable vehicle). A placebo was prepared for both brands of applesauce following the procedure described above, omitting the addition of the apalutamide tablets, and tested according to the UHPLC Procedure described above.

Table AS-1 below lists the measured assay results for the different applesauce brands and concentrations, as measured for the assay sample prepared immediately upon formation of a uniform mixture (T0). For each brand and concentration, three samples were tested, with the results presented individually below.

TABLE AS-1
Dosing Accuracy - Applesauce

| | Applesauce | | |
|---|---|---|---|
| Brand | Amount (mL) | Replicate | Assay (w/w %) ^{a} |
| Apalutamide Reference | N/A | N/A | 102.3 |
| Queen's Jewel (BE) | 50 | 1 | 98.6 |
| | | 2 | 100.1 |
| | | 3 | 98.6 |
| | 200 | 1 | 101.8 |
| | | 2 | 100.9 |
| | | 3 | 100.4 |
| Motts (US) | 50 | 1 | 98.3 |
| | | 2 | 100.0 |
| | | 3 | 98.5 |
| | 200 | 1 | 104.0 |
| | | 2 | 103.7 |
| | | 3 | 104.5 |

Dose accuracy limits: No individual result outside 85.0-115.0%

### Appearance of Applesauce Dispersion

The appearance of each applesauce dispersion was visually evaluated immediately after dispensing (T0), and then again after 3 hours and 6 hours, with the dispersion held at room temperature.

No significant changes were observed in the visual appearance of the applesauce dispersion over the 6 hour testing period.

### Dose Accuracy and Chromatographic Purity as a Function of Time

The dose accuracy of apalutamide in the applesauce dispersions (at 50 mL and 200 mL) was measured immediately after formation of the dispersion (at T0), and after 3 hours and 6 hours, with the dispersions held at room temperature. Dosing amounts were determined according to the UHPLC Procedure described above.

The dose accuracy results (presented in Table AS-2 below) exhibited a lowest measured assay of 98.3% and a highest measured assay of 106.6% (within the range of 85.0-115.0%).

The chemical stability of apalutamide in the applesauce dispersions (at 50 mL and 200 mL) was measured immediately after dispensing (at T0), and after 3 hours and 6 hours, with the dispersions held at room temperature. Chemical stability was measured according to the UHPLC Procedure described above.

The chemical stability of apalutamide in the applesauce dispersions is presented in Table AS-2 below. No significant changes in chromatographic purity were measured with all samples falling within the range of 85.0-115.0%. Placebo peaks were observed in all the samples early in the chromatographic run. Additionally, none of the peaks showed a stability trend, when comparing measurements at T0, 3 hours and 6 hours. The reporting threshold for specified and unspecified degradation products was ≥0.05%.

Table AS-2 below, lists the measured dose assay, as well as chemical stability, measuring w/w % assay of the known degradation product 4-[7-[6-cyano-5-(trifluoromethyl)-3-pyridinyl]-6,8-dioxo-5,7-diazaspiro[3.4]oct-5-yl]-2-fluoro-N-methyl-benzamide, any unknown degradation products meeting the measurement threshold and total degradation products.

TABLE AS-2
Dose Accuracy and Chemical Stability as a Function of Time-Applesauce

| | | | Assay (w/w %) | | | |
|---|---|---|---|---|---|---|
| | | | | Known | Unspecified | Total |
| | Volume | Time | | Degradation | Degradation | Degradation |
| | (mL) | (Hours) | Apalutamide | Product | Product^{a} | Products^{b} |
| Apalutamide | N/A | N/A | 102.3 | 0.10 | <0.05 | 0.10 |
| Reference | | | | | | |
| Queen's Jewel | 50 mL | T0 | 98.6 | 0.10 | <0.05 | 0.10 |
| (BE) | | 3 | 99.4 | 0.11 | <0.05 | 0.11 |
| | | 6 | 99.0 | 0.11 | <0.05 | 0.11 |
| | 200 mL | T0 | 101.8 | 0.12 | <0.05 | 0.12 |
| | | 3 | 100.2 | 0.12 | <0.05 | 0.12 |
| | | 6 | 102.0 | 0.12 | <0.05 | 0.12 |
| Motts (US) | 50 mL | T0 | 98.3 | 0.11 | <0.05 | 0.11 |
| | | 3 | 99.3 | 0.12 | <0.05 | 0.12 |
| | | 6 | 99.1 | 0.12 | <0.05 | 0.12 |
| | 200 mL | T0 | 104.0 | 0.13 | <0.05 | 0.13 |
| | | 3 | 105.7 | 0.14 | <0.05 | 0.14 |
| | | 6 | 106.6 | 0.14 | <0.05 | 0.14 |

^{a}Unspecified degradation products ≥0.05% (reporting threshold)
^{b}Sum total of all degradation products ≥0.05% (totals are calculated on unrounded results)

The results above indicate that a dose of four 60 mg tablets of apalutamide may be accurately dispersed in between about 50 mL and about 200 mL applesauce, with no observed stability changes when held for up to 6 hours at room temperature.

### Example 5: Green Tea (Extract)

### Preparation of Green Tea Extract Dispersions

The following steps were followed to disperse tablets of apalutamide in green tea extract, over 30 min. Using a measuring cylinder, 50 mL or 200 mL of green tea extract (prepared as described above) was placed into a 250 mL glass beaker and the temperature of the mixture was recorded. Four 60 mg tablets of apalutamide were removed from their packaging and placed in the beaker containing the green tea extract. The mixture was gently mixed in the container with a spatula for 10 s and the time recorded. The holding time of the sample was calculated against the moment the tablets were added to the mixture. After 15 minutes, the mixture was again gently mixed with a spatula for 10 seconds and the time recorded. After an additional 15 minutes, the mixture was again gently mixed with a spatula at least 10 seconds until a uniform mixture was formed and the time recorded. The temperature of the mixture was measured and documented; and the visual appearance of the mixture was documented.

The following steps were followed to disperse tablets of apalutamide in green tea extract, over 15 min. Using a measuring cylinder, 50 mL or 200 mL of green tea extract (prepared as described above) was placed into a 250 mL glass beaker and the temperature of the mixture was recorded. Four 60 mg tablets of apalutamide were removed from their packaging and placed in the beaker containing the green tea. The mixture was gently mixed in the container with a spatula for 10 s and the time recorded. The holding time of the sample was calculated against the moment the tablets were added to the mixture. After 15 minutes, the mixture was again gently mixed with a spatula for 10 seconds and the time recorded. The temperature of the mixture was measured and documented; and the visual appearance of the mixture was documented.

### Dose Accuracy

Assay solution samples for dose accuracy were prepared as follows. The contents of the glass beaker were emptied into a 1000 mL volumetric flask using a spatula. 60 mL of water were added to the empty glass beaker and gently swirled to rinse it. The first rinse was added to the volumetric flask. An additional 60 mL of water was added to the empty glass beaker and gently swirled to rinse it. The second rinse was added to the volumetric flask. The mixture in the volumetric flask was mixed well by vigorous manual shaking.

Approximately 120 mL of acetonitrile was added to the volumetric flask by graduated cylinder. Approximately 200 mL of dilution solvent (50:50 vol:vol water:acetonitrile) was then added to the volumetric flask by graduated cylinder. The mixture was shaken mechanically for 30 minutes. Additional dilution solvent was added up to volume in the 1000 mL flask, and the mixture was shaken vigorously, manually. The mixture was allowed to equilibrate to ambient temperature over at least 2 hours. Just before filtering, the mixture in the volumetric flask was vigorously shaken manually. The sample solution was then filtered through a chemical resistant 0.2 um filter. The first 3 mL of filtrate was discarded into a waste container (not back into the volumetric flask). The remaining filtrate was used to fill the auto-sampler vial to the appropriate height.

The final nominal assay solution sample concentration of apalutamide was 0.24 mg/mL. The assay solution samples were analyzed for dosing amount according the UHPLC Procedure described above. Placebo solution was prepared as described above, excluding the addition of the apalutamide tablets. The placebo solution was prepared to determine if observed peaks are product related or related to the used green tea extract. Each green tea extract dispersion (50 ml and 200 mL, 15 min and 30 min mixing procedure each) was measured in triplicate. A total of 12 results were generated, which are reported in Table GT-1, below, individually. Assay results are presented relative to the determined assay of the intact apalutamide tablets. The assay of the tablet sample (i.e. Reference Solution A) was determined as a comparison for all assay values of the dispersed drug product. The reference sample (Reference Solution A) preparation was as described in the UHPLC method description above.

TABLE GT-1
Dose Accuracy-Green Tea

| | | | | | Green Tea | |
|---|---|---|---|---|---|---|
| | | | | | Temperature (° C.) | |
| Brand/ | | | | | Before | After |
| Dispersing | Volume | | Assay | Visual | adding | completion of |
| time | (mL) | Replicate | (w/w %) | Appearance | tablets | dispersion |
| Apalutamide | N/A | N/A | 102.0 | N/A | N/A | N/A |
| Reference | | | | | | |
| Tetley green tea | 50 | 1 | 100.9 | Slightly yellowish | 45.8 | 23.6 |
| extract/30 min | | | | green tea | | |
| | | 2 | 101.3 | Slightly yellowish | 41.5 | 23.2 |
| | | | | green tea | | |
| | | 3 | 101.1 | Slightly yellowish | 38.2 | 22.7 |
| | | | | green tea | | |
| | 200 | 1 | 99.7 | Slightly yellowish | 51.6 | 38.6 |
| | | | | green tea | | |
| | | 2 | 99.9 | Slightly yellowish | 49.8 | 37.1 |
| | | | | green tea | | |
| | | 3 | 101.5 | Slightly yellowish | 48.4 | 35.8 |
| | | | | green tea | | |
| Tetley green tea | 50 | 1 | 101.0 | Slightly yellowish | 45.7 | 27.2 |
| extract/15 min | | | | green tea | | |
| | | 2 | 100.9 | Slightly yellowish | 41.5 | 26.8 |
| | | | | green tea | | |
| | | 3 | 102.1 | Slightly yellowish | 38.2 | 26.2 |
| | | | | green tea | | |
| | 200 | 1 | 102.3 | Slightly yellowish | 51.8 | 44.5 |
| | | | | green tea | | |
| | | 2 | 100.1 | Slightly yellowish | 49.5 | 42.3 |
| | | | | green tea | | |
| | | 3 | 100.9 | Slightly yellowish | 48.0 | 40.1 |
| | | | | green tea | | |

Dose accuracy limits: 85%-115% in the individual sample

### Appearance of Green Tea Dispersion

The appearance of each green tea extract dispersion was visually evaluated immediately after dispensing (T0), and then again after 3 hours and 6 hours, with the dispersion held at room temperature.

The visual appearance of the green tea extract after dispersing the tablets was found to be changed from yellowish brown (placebo) to slightly yellowish (dispersed mixture). No significant changes were observed over the 6 hour testing period.

### Dose Accuracy and Chromatographic Purity as a Function of Time

The dose accuracy of apalutamide in the green tea extract dispersions prepared using the 30 min dispersing time (at 50 mL and 200 mL) was measured immediately after formation of the dispersion (at T0), and after 3 hours and 6 hours, with the dispersions held at room temperature. Samples were tested in duplicate. Dosing amounts were determined according to the UHPLC Procedure described above.

A placebo solution sample was prepared according to the procedure described above, omitting the addition of the apalutamide tablets and tested according to the UHPLC Procedure described above. The placebo solution sample was used to determine if peaks were apalutamide or related to the green tea extract.

The dose accuracy results for the green tea extract dispersions as w/w % are presented in Table GT-2 below, ranged from the lowest measured assay of 99.7% to the highest measured assay of 102.3% (within the range of 85.0-115.0%). No significant changes were observed during 6 hour study period.

The chemical stability of apalutamide in the green tea extract dispersions (at 50 mL and 200 mL), prepared using the 30 min dispersion procedure, was measured immediately after dispensing (at T0), and after 3 hours and 6 hours, with the dispersions held at room temperature. Chemical stability was measured according to the UHPLC Procedure described above. The chemical stability results for the green tea extract dispersions are presented in Table GT-2 below, including w/w % assay for the known degradation product 4-[7-[6-cyano-5-(trifluoromethyl)-3-pyridinyl]-6,8-dioxo-5,7-diazaspiro[3.4]oct-5-yl]-2-fluoro-N-methyl-benzamide, any unknown degradation products meeting the measurement threshold and total degradation products. The reporting threshold for specified and unspecified degradation products was 1:105%.

No significant changes were measured during the 6 hour study period. Placebo peaks were observed randomly in all the samples. None of the impurities showed increasing trend during the 6 hours study period.

TABLE GT-2
Dose Accuracy and Chemical Stability as a Function of Time-Green Tea

| | | | | | Chromatographic Purity | | |
|---|---|---|---|---|---|---|---|
| | | | | | (w/w %) | | |
| | | | | Apalutamide | Known | Unspecified | Total |
| | Volume | Time | | Assay | Degradation | Degradation | Degradation |
| | (mL) | (Hours) | Replicate | (w/w %) | Product | Product^{a} | Products^{b} |
| Apalutamide | N/A | N/A | N/A | 102.0 | 0.15 | <0.05 | 0.15 |
| Reference | | | | | | | |
| Tetley green | 50 | T0^{c} | 1 | 100.9 | 0.15 | <0.05 | 0.15 |
| tea | | T0^{c} | 2 | 101.3 | 0.15 | <0.05 | 0.15 |
| | | 3 | 1 | 101.9 | 0.15 | <0.05 | 0.15 |
| | | 3 | 2 | 101.3 | 0.15 | <0.05 | 0.15 |
| | | 6 | 1 | 101.8 | 0.15 | <0.05 | 0.15 |
| | | 6 | 2 | 101.3 | 0.15 | <0.05 | 0.15 |
| | 200 | T0^{c} | 1 | 99.7 | 0.15 | <0.05 | 0.15 |
| | | T0^{c} | 2 | 99.9 | 0.15 | <0.05 | 0.15 |
| | | 3 | 1 | 101.9 | 0.15 | <0.05 | 0.15 |
| | | 3 | 2 | 100.3 | 0.15 | <0.05 | 0.15 |
| | | 6 | 1 | 100.0 | 0.15 | <0.05 | 0.15 |
| | | 6 | 2 | 100.8 | 0.15 | <0.05 | 0.15 |

^{a}Unspecified degradation products ≥0.05% (reporting threshold)
^{b}Sum total of all degradation products ≥0.05% (totals are calculated on unrounded results)

The results above indicate that a dose of four 60 mg tablets of apalutamide may be accurately dispersed in between about 50 mL and about 200 mL green tea, with dispersion time of 15 min and 30 min, and with no observed stability changes when help for up to 6 hours at room temperature.

### Example 6: Orange Juice

### Preparation of Orange Juice Dispersions

The following steps were followed to disperse tablets of apalutamide in orange juice (50 mL or 200 mL). An orange juice container was shaken well and orange juice (50 mL or 200 mL) was removed to a glass beaker using a graduated cylinder. Four 60 mg tablets of apalutamide were removed from their packaging and placed in the beaker containing the orange juice. The mixture was gently mixed in the container with a spatula for 10 s and the time recorded. The holding time of the sample was calculated against the moment the tablets were added to the mixture. After 15 minutes, the mixture was again gently mixed with a spatula for 10 seconds and the time recorded. After an additional 15 minutes, the mixture was again gently mixed with a spatula at least 10 seconds until a uniform mixture was formed and the time recorded. The temperature of the mixture was measured and documented; and the visual appearance of the mixture was documented.

### Dose Accuracy

Assay solution samples for dose accuracy were prepared as follows. The contents of the glass beaker were emptied into a 1000 mL volumetric flask using a spatula. 60 mL of water were added to the empty glass beaker and gently swirled to rinse it. The first rinse was added to the volumetric flask. An additional 60 mL of water was added to the empty glass beaker and gently swirled to rinse it. The second rinse was added to the volumetric flask. The mixture in the volumetric flask was mixed well by vigorous manual shaking.

Approximately 120 mL of acetonitrile was added to the volumetric flask by graduated cylinder. Approximately 200 mL of dilution solvent (50:50 vol:vol water:acetonitrile) was then added to the volumetric flask by graduated cylinder. The mixture was shaken mechanically for 30 minutes. Additional dilution solvent was added up to volume in the 1000 mL flask, and the mixture was shaken vigorously, manually. The mixture was allowed to equilibrate to ambient temperature over at least 2 hours. Just before filtering, the mixture in the volumetric flask was vigorously shaken manually. The sample solution was then filtered through a chemical resistant 0.2 um filter. The first 3 mL of filtrate was discarded into a waste container (not back into the volumetric flask). The remaining filtrate was used to fill the auto-sampler vial to the appropriate height.

The final nominal assay solution sample concentration of apalutamide was 0.24 mg/mL. The assay solution samples were analyzed for dosing amount according to the UHPLC Procedure described above. Each sample was tested in triplicate.

A placebo solution sample was prepared according to the procedure described above, omitting the addition of the apalutamide tablets and tested according to the UHPLC Procedure described above. It was noted that the final orange juice solution was difficult to filter, and was therefore centrifuged prior to filtration. The placebo solution sample was used to determine if peaks were apalutamide or related to the orange juice.

Table OJ-1 below lists the measured assay results for the different orange preparations, as measured immediately upon formation of the dispersion. Three samples were tested for each concentration for a total of 12 results, which are reported in Table OJ-1, below, individually. Assay results are presented relative to the determined assay of Reference Solution A (apalutamide tablets in acetonitrile UHPLC solvent). The visual appearance of each of the orange juice dispersions was noted as yellowish orange.

TABLE OJ-1
Dose Accuracy-Orange Juice

| | Volume (mL) | Replicate | Assay (w/w %) |
|---|---|---|---|
| Apalutamide Reference | N/A | N/A | 102.0 |
| Minute Maid | 50 | 1 | 102.0 |
| Orange Juice | | | |
| | | 2 | 101.1 |
| | | 3 | 100.2 |
| | 200 | 1 | 100.8 |
| | | 2 | 97.5 |
| | | 3 | 98.9 |

Dose accuracy limits: 85%-115% in the individual sample

### Visual Appearance of Orange Juice Dispersions

The appearance of each orange juice dispersion was visually evaluated immediately after dispensing (T0), and then again after 3 hours and 6 hours, with the dispersion held at room temperature.

The visual appearance of the orange juice dispersions after dispersing the tablets was observed to change from orange (placebo) to yellowish orange (dispersed mixture) color. No significant changes were observed over the 6 hour testing period.

### Dose Accuracy and Chromatographic Purity as a Function of Time

The dose accuracy of apalutamide in the orange juice dispersions (at 50 mL and 200 mL) was measured immediately after formation of the dispersion (at T0), and after 3 hours and 6 hours, with the dispersions held at room temperature. Duplicate samples were prepared and tested according to the UHPLC Procedure described above. A placebo solution sample was prepared as described above, omitting the addition of the apalutamide tablets and tested according to the UHPLC Procedure described above. This solution sample and UHPLC results were used to determine if peaks were apalutamide or related to the orange juice. Placebo peak correction was applied, to correct for possible interference at the peak of interest.

The dose accuracy results, presented in Table OJ-2 below, range from the lowest measured assay of 97.5% to the highest measured assay of 102.0% (within the range of 85.0-115.0%). No significant changes were observed during the 6 hour study period.

The chemical stability of apalutamide in orange juice dispersions (at 50 mL and 200 mL) was measured immediately after dispensing (at T0), and after 3 hours and 6 hours, with the dispersions held at room temperature. Chemical stability was measured according to the UHPLC Procedure described above. The chemical stability results for the orange juice dispersions, presented in Table OJ-2 below, include w/w % assay for the known degradation product 4-[7-[6-cyano-5-(trifluoromethyl)-3-pyridinyl]-6,8-dioxo-5,7-diazaspiro[3.4]oct-5-yl]-2-fluoro-N-methyl-benzamide, any unknown degradation products meeting the measurement threshold and total degradation products. The reporting threshold for specified and unspecified degradation products was 0.05%.

No significant changes were measured during the 6 hour study period. Placebo peaks were observed randomly in all the samples. A placebo peak was observed at the retention time of a known process impurity-4-((1-((6-cyano-5-(trifluoromethyl)pyridin-3-yl)carbamoyl)cyclobutyl)amino)-2-fluoro-N-methylbenzamide, however this impurity was not observed in the placebo sample. None of the impurities showed an increasing trend during the tested in use period of 6 hours.

TABLE OJ-2
Dose Accuracy and Chemical Stability as a Function of Time-Orange Juice

| | | | | | Chromatographic Purity | | |
|---|---|---|---|---|---|---|---|
| | | | | | (w/w %) | | |
| | | | | Apalutamide | Known | Unspecified | Total |
| | Volume | Time | | Assay | Degradation | Degradation | Degradation |
| | (mL) | (Hours) | Replicate | (w/w %) | Product | Product^{a} | Products^{b} |
| Apalutamide | N/A | N/A | N/A | 102.0 | 0.15 | <0.05 | 0.15 |
| Reference | | | | | | | |
| Minue Maid | 50 | T0^{c} | 1 | 102.0 | 0.16 | <0.05 | 0.16 |
| ornage juice | | T0^{c} | 2 | 101.1 | 0.17 | <0.05 | 0.17 |
| | | 3 | 1 | 100.2 | 0.17 | <0.05 | 0.17 |
| | | 3 | 2 | 101.0 | 0.16 | <0.05 | 0.16 |
| | | 6 | 1 | 101.6 | 0.16 | <0.05 | 0.16 |
| | | 6 | 2 | 100.8 | 0.16 | <0.05 | 0.16 |
| | 200 | T0^{c} | 1 | 100.8 | 0.17 | <0.05 | 0.17 |
| | | T0^{c} | 2 | 97.5 | 0.16 | <0.05 | 0.16 |
| | | 3 | 1 | 99.6 | 0.16 | <0.05 | 0.16 |
| | | 3 | 2 | 99.0 | 0.17 | <0.05 | 0.17 |
| | | 6 | 1 | 99.8 | 0.16 | <0.05 | 0.16 |
| | | 6 | 2 | 101.7 | 0.16 | <0.05 | 0.16 |

^{a}Unspecified degradation products ≥0.05% (reporting threshold)
^{b}Sum total of all degradation products ≥0.05% (totals are calculated on unrounded results)

The results above indicate that a dose of four 60 mg tablets of apalutamide may be accurately dispersed into between about 50 mL and about 200 mL orange juice, and kept at room temperature or cold condition; and that no stability changes are observed when said dispersions are held for 6 hours at room temperature.

### Example 7: Yogurt

### Preparation of Yogurt Dispersions

The following steps were followed to disperse tablets of apalutamide in yogurt. Yogurt was used either cold or at room temperature. Cold yogurt was removed from the refrigerator and used directly for dose preparation without equilibration at room temperature. Room temperature yogurt was removed from the refrigerator and equilibrated at room temperature before being used for dose preparation. Cold or room temperature yogurt (50 g or 200 g) was added to a tared glass beaker. Four 60 mg tablets of apalutamide were removed from their packaging and placed in the beaker containing the yogurt. The mixture was gently mixed in the container with a spatula for 10 seconds and the time recorded. The holding time of the sample was calculated against the moment the tablets were added to the mixture. After 15 minutes, the mixture was again gently mixed with a spatula for 10 seconds and the time recorded. After an additional 15 minutes, the mixture was again gently mixed with a spatula at least 10 seconds until a uniform mixture was formed and the time recorded. The temperature of the mixture was measured and documented; and the visual appearance of the mixture was documented.

### Dose Accuracy

Assay solution samples for dose accuracy were prepared as follows. The contents of the glass beaker were emptied into a 1000 mL volumetric flask using a spatula. 60 mL of water were added to the empty glass beaker and gently swirled to rinse it. The first rinse was added to the volumetric flask. An additional 60 mL of water was added to the empty glass beaker and gently swirled to rinse it. The second rinse was added to the volumetric flask. The mixture in the volumetric flask was mixed well by vigorous manual shaking.

Approximately 350 mL of dilution solvent (50:50 vol:vol water:acetonitrile) was then added to the volumetric flask by graduated cylinder. The mixture was shaken mechanically for 30 minutes. Additional dilution solvent was added up to volume in the 1000 mL flask, and the mixture was shaken vigorously, manually. The mixture was allowed to equilibrate to ambient temperature over at least 2 hours. Just before filtering, the mixture in the volumetric flask was vigorously shaken manually. The sample solution was then filtered through a chemical resistant 0.2 um filter. The first 3 mL of filtrate was discarded into a waste container (not back into the volumetric flask). The remaining filtrate was used to fill the auto-sampler vial to the appropriate height.

The final nominal assay solution sample concentration of apalutamide was 0.24 mg/mL. The assay solution samples were analyzed for dosing amount according to the UHPLC Procedure described above. Each sample was tested in triplicate. A placebo solution sample was prepared according to the procedure described above, omitting the addition of the apalutamide tablets and tested according to the UHPLC Procedure described above. This solution sample and UHPLC results were used to determine if peaks were apalutamide or related to the yogurt. The final yogurt solution was difficult to filter, and was therefore centrifuged, prior to filtration.

Table Y-1 below lists the measured assay results for the different yogurt preparations, as measured immediately upon formation of the dispersion. Three samples were tested for each dispersion for a total of 12 results, which are reported in Table Y-1, below, individually. Assay results are presented relative to the determined assay of Reference Solution A (apalutamide tablets in acetonitrile UHPLC solvent). The visual appearance of each of the yogurt dispersion was noted as slightly yellowish.

TABLE Y-1
Dose Accuracy - Yogurt

| | Yogurt | | |
|---|---|---|---|
| | Amount (g) | Replicate | Assay (w/w %) |
| Apalutamide Reference | N/A | N/A | 102.6 |
| Epigamia Greek yogurt | 50 (cold) | 1 | 100.2 |
| | | 2 | 101.3 |
| | | 3 | 100.5 |
| | 200 (room | 1 | 95.9 |
| | temperature) | 2 | 95.7 |
| | | 3 | 95.1 |
| Gowardhan yogurt | 50 (cold) | 1 | 100.4 |
| | | 2 | 100.4 |
| | | 3 | 99.8 |
| | 200 (room | 1 | 97.0 |
| | temperature) | 2 | 96.5 |
| | | 3 | 95.4 |

Dose accuracy range: 85%-115% in the individual sample

### Visual Appearance of Yogurt Dispersions

The appearance of each yogurt dispersion was visually evaluated immediately after dispensing (T0), and then again after 3 hours and 6 hours, with the dispersion held at room temperature.

The visual appearance of the yogurt dispersions was observed to change from white (placebo) to slightly yellowish (dispersed mixture). No significant changes in visual appearance were observed over the 6 hour testing period.

### Dose Accuracy and Chromatographic Purity as a Function of Time

The dose accuracy of apalutamide in the yogurt dispersions (at 50 g and 200 g) was measured immediately after formation of the dispersion (at T0), and after 3 hours and 6 hours, with the dispersions held at room temperature. Single samples were prepared and tested according to the UHPLC Procedure described above. A placebo solution sample was prepared according to the procedure described above, omitting the addition of the apalutamide tablets and tested according to the UHPLC Procedure described above. The placebo solution sample and UHPLC results were used to determine if peaks were apalutamide or related to the yogurt Placebo peak correction was applied, to correct for possible interference at the peak of interest.

The dose accuracy results, presented in Table Y-2 below, ranged from the lowest measured assay of 95.1% to the highest measured assay of 101.3% (within the range of 85.0-115.0%). It was observed that the measured assay values for the 200 g preparations were slightly on lower side than the other samples, but nonetheless were within acceptance criteria. It is hypothesized that this is an analytical artifact of the yogurt on the sample preparation during the dilution of the analytical UHPLC method. No significant changes were observed during the 6 hour study period.

The chemical stability of apalutamide in yogurt dispersions (at 50 g and 200 g) was measured immediately after dispensing (at T0), and after 3 hours and 6 hours, with the dispersions held at room temperature. Chemical stability was measured according to the UHPLC Procedure described above. The chemical stability results for the yogurt dispersions, presented in Table Y-2 below, include w/w % assay for the known degradation product 4-[7-[6-cyano-5-(trifluoromethyl)-3-pyridinyl]-6,8-dioxo-5,7-diazaspiro[3.4]oct-5-yl]-2-fluoro-N-methyl-benzamide, any unknown degradation products meeting the measurement threshold and total degradation products. The reporting threshold for specified and unspecified degradation products was 0.05%.

No significant changes were measured during the 6 hour study period. Placebo peaks were observed randomly in all the samples. A placebo peak was observed at the retention time of a known process impurity-4-((1-((6-cyano-5-(trifluoromethyl)pyridin-3-yl)carbamoyl)cyclobutyl)amino)-2-fluoro-N-methylbenzamide, however this impurity was not observed in the placebo sample. None of the impurities showed an increasing trend during the tested in use period of 6 hours.

TABLE Y-2
Dose Accuracy and Chemical Stability as a Function of Time-Yogurt

| | | | Chromatographic Purity | | | |
|---|---|---|---|---|---|---|
| | | | (w/w %) | | | |
| | | | Apalutamide | Known | Unspecified | Total |
| | Volume | Time | Assay | Degradation | Degradation | Degradation |
| | (mL) | (Hours) | (w/w %) | Product | Product^{a} | Products^{b} |
| Apalutamide | N/A | N/A | 102.6 | 0.15 | <0.05 | 0.15 |
| Reference | | | | | | |
| Epigamia | 50 | T0 | 100.2 | 0.16 | <0.05 | 0.16 |
| Greek yogurt | | 3 | 99.6 | 0.15 | <0.05 | 0.15 |
| | | 6 | 100.7 | 0.16 | <0.05 | 0.16 |
| | 200 | T0 | 95.9 | 0.13 | <0.05 | 0.13 |
| | | 3 | 95.6 | 0.12 | <0.05 | 0.12 |
| | | 6 | 95.0 | 0.14 | <0.05 | 0.14 |
| Gowardhan | 50 | T0 | 100.4 | 0.16 | <0.05 | 0.16 |
| yogurt | | 3 | 100.7 | 0.15 | <0.05 | 0.15 |
| | | 6 | 100.4 | 0.15 | <0.05 | 0.15 |
| | 200 | T0 | 97.0 | 0.14 | <0.05 | 0.14 |
| | | 3 | 95.5 | 0.12 | <0.05 | 0.12 |
| | | 6 | 96.6 | 0.14 | <0.05 | 0.14 |

^{a}Unspecified degradation products ≥0.05% (reporting threshold)
^{b}Sum total of all degradation products ≥0.05% (totals are calculated on unrounded results)

The results indicate that the dose of four 60 mg tablets apalutamide may be accurately dispersed in between about 50 g and about 200 g yogurt when kept at room temperature and cold condition. For the dispersions in yogurt, no stability changes were observed when the mixture was held for 6 hours at room temperature.

### Example 8—FDA Product Label

On Feb. 14, 2018 the FDA approved the drug product label for ERLEADA™ (apalutamide), which will be the reference listed drug for apalutamide. The following amended label, which includes administration of apalutamide as a mixture in applesauce, was approved by the FDA in July 2020.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

What is claimed is:

1. A method of treating prostate cancer comprising administering apalutamide to a male human with a prostate cancer; wherein the apalutamide is dispersed in a suitable vehicle.

2. A method as in claim 1, wherein the prostate cancer is non-metastatic castration-resistant prostate cancer.

3. A method as in claim 1, wherein the apalutamide is administered in an amount between about 60 mg and about 240 mg.

4. A method as in claim 1, wherein the apalutamide is administered in an amount of about 240 mg.

5. A method as in claim 1, wherein the apalutamide is administered to a fasting male human.

6. A method as in claim 1, wherein the apalutamide is dispersed by admixing four 60 mg solid tablets of apalutamide in between about 50 ml and about 200 mL or between about 50 g and about 200 g of the suitable vehicle.

7. A method as in claim 1, wherein the apalutamide dispersed in the suitable vehicle is administered over a period of about 5 minutes.

8. A method as in claim 1, wherein apalutamide dispersed in the suitable vehicle is administered daily.

9. The method as in claim 1, wherein apalutamide dispersed in the suitable vehicle is administered orally on a continuous daily dosage schedule.

10. A method as in claim 1, wherein a male human has said non-metastatic castration-resistant prostate cancer and has a prostate-specific antigen doubling time (PSADT) that is less than or equal to 10 months.

11. A method as in claim 1, wherein administration of the apalutamide provides an increase in the metastasis-free survival of a male human.

12. A method as in claim 1, wherein administration of the apalutamide provides improved anti-tumor activity as measured by time to metastasis (TTM), progression-free survival (PFS) rate, time to symptomatic progression, overall survival (OS) rate, or time to initiation of cytotoxic chemotherapy.

13. A method as in claim 1, wherein administration of the apalutamide results in no more than a grade 3 adverse event.

14. A method as in claim 1, wherein a male human having said non-metastatic castration-resistant prostate cancer is treatment naïve.

15. A method as in claim 1, wherein a male human having said prostate cancer has received at least one prior therapy for the treatment of cancer.

16. A method as in claim 18, wherein the prior therapy for the treatment of cancer is bicalutamine or flutamide.

17. A method as in claim 1, wherein apalutamide is co-administered with one or more additional cancer treatments.

18. A method as in claim 1, wherein the amount of apalutamide administered is reduced when co-administered with one or more of:
(a) a CYP2C8 inhibitor, preferably gemfibrozil or clopidogrel; or
(b) a CYP3A4 inhibitor, preferably ketoconazole or ritonavir.

19. A method as in claim 1, wherein apalutamide is not co-administered with:
(a) medications that are primarily metabolized by CYP3A4, preferably darunavir, felodipine, midazolam or simvastatin;
(b) medications that are primarily metabolized by CYP2C19, preferably diazepam or omeprazole;
(c) medications that are primarily metabolized by CYP2C9, preferably warfarin or phenytoin; or
(d) medications that are substrates of UGT, preferably levothyroxine or valproic acid.

20. A method as in claim 1, wherein apalutamide is not co-administered with:
(a) medications that are P-gp substrates, preferably fexofenadine, colchicine, dabigatran etexilate or digoxin; or
(b) BCRP/OATP1B1 substrates, preferably lapatinib, methotrexate, rosuvastatin, or repaglinide.

21. A method as in claim 1, wherein the suitable vehicle is a fluid or semi-solid.

22. A method as in claim 1, wherein the suitable vehicle is a liquid.

23. A method as in claim 1, wherein the suitable vehicle is a liquid selected from the group consisting of water, tea, coffee, milk, coconut milk, fruit juice, lemonade, plant based milk, sports drink and electrolyte drink.

24. A method as in claim 1, wherein the suitable vehicle is fruit juice or tea.

25. A method as in claim 1, wherein the suitable vehicle is fruit juice and wherein the fruit juice is orange juice.

26. A method as in claim 1, wherein the suitable vehicle is tea and wherein the tea is green tea.

27. A method as in claim 1, wherein the suitable vehicle is a semi-solid.

28. A method as in claim 1, wherein the suitable vehicle is a semi-solid selected from the group consisting of applesauce, fruit puree, yogurt and jam.

29. A method as in claim 1, wherein the suitable vehicle is a semi-solid, and wherein the semi-solid is applesauce or yogurt.

30. A method of treatment as in claim 1, comprising the steps of
(a) mixing apalutamide in a suitable vehicle; to yield an apalutamide-vehicle mixture;
(b) administering the apalutamide-vehicle mixture to a patient in need thereof; wherein the apalutamide-vehicle mixture is administered orally.

31. A method of treatment as in claim 1, comprising the steps of
(a) placing one to four whole, 60 mg tablets of apalutamide in 4 ounces (120 mL) of applesauce and stirring to form a first mixture;
(b) holding the first mixture without stirring for 15 minutes; and then stirring the first mixture; to yield a second mixture;
(c) holding the second mixture without stirring for an additional 15 minutes; and then stirring the second mixture until the tablets are mixed well with no chunks remaining; to yield an apalutamide-applesauce mixture;
(d) administering the apalutamide-vehicle mixture to a patient in need thereof; wherein the apalutamide-vehicle mixture is administered orally.

32. A method of treatment as in claim 1, comprising the steps of
(a) in a container, placing one to four whole tablets of apalutamide in 4 ounces (120 mL) of applesauce and stirring to form a first mixture;
(b) holding the first mixture without stirring for 15 minutes; and then stirring the first mixture; to yield a second mixture;
(c) holding the second mixture without stirring for an additional 15 minutes, and then stirring the second mixture until the tablets are mixed well with no chunks remaining; to yield an apalutamide-applesauce mixture;
(d) swallowing the apalutamide-applesauce mixture; wherein the apalutamide-applesauce mixture is swallowed within one hour of preparation;
(e) rinsing the container with 2 ounces (60 mL) of water and immediately drinking the contents; to yield a once rinsed container;
(f) rinsing the once rinsed container with 2 ounces (60 mL) of water and immediately drinking the contents.

33. A method of treatment as in claim 1, comprising the steps of
(a) adding one to four whole, 60 mg tablets of apalutamide into between about 50 ml and about 200 ml or between 50 g and 200 g of a suitable vehicle; to yield a first mixture;
(b) mixing the first mixture for 10 seconds; to yield a second mixture;
(c) holding the second mixture without mixing for 15 min; to yield a third mixture;
(d) mixing the third mixture for 10 seconds; to yield a fourth mixture;
(e) repeating Steps (c) and (d) until a visually uniform mixture is formed;
(f) administering the apalutamide-vehicle mixture to a patient in need thereof; wherein the apalutamide-vehicle mixture is administered orally.

34. A method of treating non-metastatic castration-resistant prostate cancer comprising administering apalutamide to a male human with a non-metastatic castration-resistant prostate cancer as described herein.
